(19) United States
(12) Patent Application Publication
(10) Pub. No.: US 2026/0284028 A1
(43) Pub. Date: Sep. 24, 2026

(54) FUSED BICYCLIC HETEROCYCLYL COMPOUNDS AS CCR4 MODULATORS

(71) Applicant: AURIGENE ONCOLOGY LIMITED, Bangalore (IN)

(72) Inventors: Chandrasekhar ABBINENI, Hyderabad (IN); Susanta SAMAJDAR, Bangalore (IN); Krishna Chaitanya TALLURI, Hyderabad (IN); Subhendu MUKHERJEE, Bangalore (IN); Chandrasekhar V MIDUTURU, Cambridge, MA (US)

(21) Appl. No.: 19/480,267

(22) PCT Filed: May 2, 2024
(86) PCT No.: PCT/IB2024/054242

§ 371 Date: Oct. 30, 2025

(30) Foreign Application Priority Data
May 2, 2023 (IN) 202341031205
Dec. 9, 2023 (IN) 202341084043

(51) Int. Cl. A61K 31/454; A61K 31/4545; A61K 31/501; A61K 31/506; A61K 31/519; A61K 31/53; C07D 401/14; C07D 413/14; C07D 417/14; C07D 498/04; C07D 513/04

(52) U.S. Cl. CPC A61K 31/454; A61K 31/4545; A61K 31/501; A61K 31/506; A61K 31/519; A61K 31/53; C07D 401/14; C07D 413/14; C07D 417/14; C07D 498/04; C07D 513/04

(57) ABSTRACT

The present disclosure provides fused bicyclic heterocyclyl compounds and their derivatives of formula (I), which are therapeutically useful as CCR4 modulators. These compounds are useful in the treatment and/or prevention of diseases and/or disorders responsive to the modulation of CCR4 activity. Compounds of the present disclosure are especially useful for treating cancer and inflammatory diseases and disorders. The present disclosure also provides processes for preparation of the compounds and pharmaceutical formulations comprising at least one of the compounds of formula (I) or a pharmaceutically acceptable salt or a stereoisomer thereof.

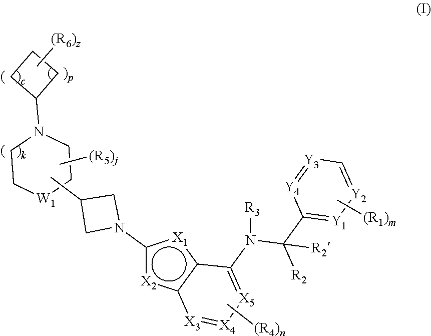

### CROSS REFERENCE TO RELATED APPLICATIONS

The present application claims priority to and the benefit of Indian Patent Application Nos.: IN 202341031205, filed on May 2, 2023, and IN 202341084043, filed on Dec. 9, 2023, which applications are incorporated herein by reference in their entireties.

### TECHNICAL FIELD

The present application is directed to fused bicyclic heterocyclyl compounds and their derivatives of formula (I) as CCR4 modulators, useful for the treatment of cancer and inflammatory diseases or disorders. The disclosure also provides pharmaceutically acceptable compositions comprising compounds of the present application and methods of using said compositions in the treatment of diseases associated with CCR4.

### BACKGROUND

Innate and adaptive immune responses and their coordinated interplay are essential to maintain homeostasis and for effective immune surveillance against pathogens and inflammatory diseases. The key mediators of the immune system include the immune cells and the cytokines produced by them. Chemokines are a family of small, secreted cytokines that control several processes such as cellular adhesion, localization, migration and cell-cell interaction (Clemens Esche, J Invest Dermatol. 2005 October; 125(4):615-28).

Inflammatory chemokines secreted by various types of cells control the recruitment of inflammatory effector cells such as leukocytes in response to infection, inflammation, tissue injury and in the tumor microenvironment (Anna E Vilgelm, Front Immunol. 2019 Feb. 27; 10:333). They exert their effect through the activation of chemokine receptors on the cell surface of the effector cells. Chemokine receptors belong to the large family of G-protein coupled receptors (GPCR). Cells expressing the chemokine receptors migrate in the direction of the respective chemokine gradient secreted by the tissue resident cells. CCR4 (C—C chemokine receptor) is an important member of the GPCR family that plays a critical role in the migration of immune cells in response to inflammatory chemokines CCL22 and CCL17 (Hongyi Li, MedComm (2020). 2022 Jun. 8; 3(2):e147).

Tumor cells and tumor-associated macrophages secrete CCL22 leading to the recruitment of CCR4 expressing regulatory T cells (Tregs) in the tumor microenvironment. This increased infiltration of Tregs leads to an immune-suppressive tumor microenvironment and is associated with poor prognosis (Osamu Yoshie, Cancers (Basel). 2021 Nov. 4; 13(21):5542).

Thus, CCR4 is implicated in the pathology of various immune-associated disorders. Effective modulation of CCR4 is hence crucial for the therapeutic intervention of inflammatory disorders and cancer.

### SUMMARY

Provided herein are compounds of formula (I) and pharmaceutical compositions thereof, which are capable of modulating CCR4.

In one aspect of the present disclosure, it comprises compounds of formula (I):

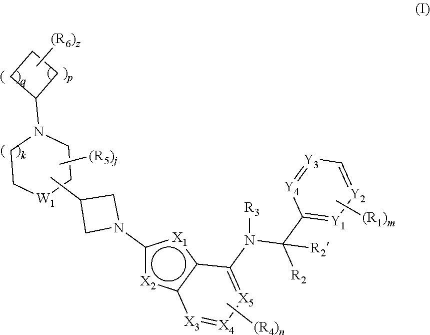

or a pharmaceutically acceptable salt thereof or a stereoisomer thereof;
wherein,
W₁ is C, N or O;
X₁ and X₂ are each independently CH, N, NRₓ, O, S or —S(O)—;
X₃, X₄ and X₅ are each independently C or N;
Rₓ is hydrogen or C₁-C₆ alkyl;
Y₁, Y₂, Y₃, and Y₄ are each independently C or N;
R₁ at each occurrence is independently C₁-C₆ alkyl, C₂-C₆ alkenyl, C₂-C₆ alkynyl, halo, cyano, amino, nitro, —OR₁ₐ, —C(O)R_{1b} or C₃-C₆ cycloalkyl; wherein each of C₁-C₆ alkyl, C₂-C₆ alkenyl, C₂-C₆ alkynyl, and C₃-C₆ cycloalkyl is optionally substituted with one or more substituents independently selected from halo, hydroxy and C₁-C₆ alkoxy;
R₂ and R₂′ are each independently hydrogen, C₁-C₆ alkyl or halo;
R₃ is hydrogen or C₁-C₆ alkyl;
R₄ at each occurrence is independently C₁-C₆ alkyl, C₂-C₆ alkenyl, C₂-C₆ alkynyl, halo, cyano, amino, nitro, —OR₄ₐ, —C(O)R_{4b}, or C₃-C₆ cycloalkyl; wherein each of C₁-C₆ alkyl, C₂-C₆ alkenyl, C₂-C₆ alkynyl, and C₃-C₆ cycloalkyl is optionally substituted with one or more substituents independently selected from one or more halo, hydroxy and C₁-C₆ alkoxy;
R₅ at each occurrence is independently halo, C₁-C₆ alkyl, C₁-C₆ haloalkyl, C₁-C₆ alkoxy or —C(O)R₅ₐ;
R₆ at each occurrence is independently C₁-C₆ alkyl, halo, C₁-C₆ haloalkyl, cyano, —C(O)R₆ₐ, —(C₁-C₃ alkyl)C(O)R₆ₐ or —OR_{6b};
R₁ₐ and R₄ₐ are each independently hydrogen, halo, C₁-C₆ alkyl, C₁-C₆ haloalkyl, C₃-C₆ cycloalkyl or C₃-C₆ heterocycloalkyl;
R_{1b}, R_{4b} and R₅ₐ are each independently hydrogen, C₁-C₆ alkyl, hydroxy, amino, C₁-C₆ alkylamino, C₁-C₆ haloalkyl, C₃-C₆ cycloalkyl or C₃-C₆ heterocycloalkyl;
R₆ₐ is hydrogen, hydroxy, C₁-C₆ alkyl, C₁-C₆ haloalkyl, C₁-C₆ alkoxy, C₁-C₆ haloalkoxy, amino, C₁-C₆ alkylamino or C₃-C₆ cycloalkyl;
R_{6b} is hydrogen, C₁-C₆ alkyl, C₁-C₆ haloalkyl or C₃-C₆ cycloalkyl;
‘m’ is selected from 0 to 5;
‘n’ is selected from 0 to 3;
‘j’ is selected from 0 to 4;
‘k’ is selected from 0 to 2;
‘p’ and ‘q’ are each independently selected from 1 and 2; and
‘z’ is selected from 0 to 4.

In yet another aspect, the present application provides pharmaceutical compositions comprising a compound of formula (I) or a pharmaceutically acceptable salt thereof and at least one pharmaceutically acceptable excipient (such as a pharmaceutically acceptable carrier or diluent).

In yet another aspect, the present application relates to the preparation of compounds of formula (I).

In yet another aspect of the present application, provided herein are compounds of formula (I), which are capable of modulating CCR4 and therapeutic use thereof.

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated to facilitate the understanding of the present disclosure.

As used herein, unless otherwise defined the term “alkyl” alone or in combination with other term(s) means saturated aliphatic hydrocarbon chains, including C₁-C₁₀ straight or C₁-C₁₀ branched alkyl groups. Examples of “alkyl” include but are not limited to methyl, ethyl, propyl, isopropyl, butyl, isobutyl, sec-butyl, tert-butyl, isopentyl or neopentyl and the like.

As used herein, unless otherwise defined the term “alkenyl” alone or in combination with other term(s) means unsaturated aliphatic hydrocarbon chains, including C₁-C₁₀ straight or C₁-C₁₀ branched alkyl groups. An alkenyl may include more than one double bond and/or one or more triple bonds in addition to the one or more double bonds. Examples of “alkenyl” include but are not limited to vinyl, 2-propenyl, 2-isopentenyl, 2-(butadienyl), 2,4-pentadienyl, 3-(1,4-pentadienyl) and the like.

As used herein, unless otherwise defined the term “alkynyl” alone or in combination with other term(s) means unsaturated aliphatic hydrocarbon chains, including C₁-C₁₀ straight or C₁-C₁₀ branched alkyl groups. An alkynyl may include more than one triple bond and/or one or more double bonds in addition to the one or more triple bonds. Examples of “alkynyl” include but are not limited to ethynyl, 1- and 3-propynyl, 3-butynyl and the like.

As used herein, the term “halo” or “halogen” alone or in combination with other term(s) means fluorine, chlorine, bromine or iodine.

As used herein, the term “haloalkyl” means alkyl substituted with one or more halogen atoms, wherein the alkyl groups are as defined above. The term “halo” is used herein interchangeably with the term “halogen” means F, Cl, Br or I. Examples of “haloalkyl” include but are not limited to fluoromethyl, difluoromethyl, chloromethyl, trifluoromethyl, 2,2,2-trifluoroethyl and the like.

As used herein, the term “hydroxy” or “hydroxyl” alone or in combination with other term(s) means —OH.

As used herein, the term “hydroxyalkyl” refers to the group HO-alkyl-, wherein alkyl and hydroxy groups are as defined herein.

As used herein, the term “alkoxy” alone or in combination with other term(s) refers to the group alkyl-O— or —O-alkyl, where alkyl groups are as defined above. Exemplary C₁-C₁₀ alkyl group containing alkoxy-groups include but are not limited to methoxy, ethoxy, n-propoxy, n-butoxy, t-butoxy and the like. An alkoxy group can be unsubstituted or substituted with one or more suitable groups.

As used herein, the term “haloalkoxy” refers to an alkoxy group substituted with one or more halogen atoms (i.e., haloC.sub.1-8alkoxy). Examples of “haloalkoxy” include but are not limited to fluoromethoxy, difluoromethoxy, trifluoromethoxy, 2,2,2-trifluoroethoxy, pentafluoroethoxy, pentachloroethoxy, chloromethoxy, dichlorormethoxy, trichloromethoxy and 1-bromoethoxy.

As used herein, the term “alkoxyalkyl” refers to the group alkyl-O-alkyl-, wherein alkyl and alkoxy groups are as defined above. Exemplary alkoxyalkyl-groups include but are not limited to methoxymethyl, ethoxymethyl, methoxyethyl, isopropoxymethyl and the like.

The term “amino” or “amine” alone or in combination with other term(s) refers to a primary amine (—NH₂), secondary amine

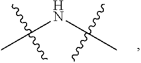 [SMILES: *N*]

wherein ‘N’ is substituted with two substituents other than hydrogen) or tertiary amine

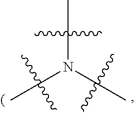 [SMILES: *N(*)*]

wherein ‘N’ is substituted with three substituents other than hydrogen) group.

As used herein, the term “alkylamino” alone or in combination with other term(s) means an amino group as defined above, substituted with one or more “alkyl” group, wherein the alkyl group and amino group is as defined above. Examples of “alkylamino” groups include but are not limited to —NHCH₃, —NHCH₂CH₃, —N(CH₃)₂, —N(CH₃)(CH₂CH₃) and the like.

As used herein, the term “cyano” refers to —CN; and the term “cyanoalkyl” refers to alkyl substituted with —CN; wherein the alkyl groups are as defined above.

As used herein, the term “nitro” refers to —NO₂; As used herein the term “cycloalkyl” alone or in combination with other term(s) means —C₃-C₁₀ saturated cyclic hydrocarbon ring. A cycloalkyl may be a single ring, which typically contains from 3 to 7 carbon ring atoms. Examples of single-ring cycloalkyls include but are not limited to cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused and spirocyclic carbocyclyls and the like.

As used herein, the term “aryl” is unsubstituted or substituted monocyclic, bicyclic or polycyclic aromatic hydrocarbon ring system of about 6 to 14 carbon atoms. Examples of a C₆-C₁₄ aryl group include, but are not limited to phenyl, naphthyl, anthryl, tetrahydronaphthyl, fluorenyl, indanyl, biphenylenyl and acenaphthyl. An aryl group may be unsubstituted or substituted with one or more suitable groups.

As used herein, the term “carbocyclyl” alone or in combination with other term(s) includes both “cycloalkyl” and “aryl” groups which are as defined above. Examples of “carbocyclyl” include, but are not limited to cyclopropyl, cyclobutyl, cyclopentyl, cyclohexyl, phenyl and naphthyl.

The term “heterocycloalkyl” refers to a non-aromatic, saturated or partially saturated monocyclic or polycyclic ring system of 3 to 15 members having at least one heteroatom or heterogroup selected from O, N, S, S(O), S(O)₂, NH or C(O) with the remaining ring atoms being independently selected from the group consisting of carbon, oxygen, nitrogen and sulfur.

A monocyclic heterocycloalkyl may typically contain 4 to 7 ring atoms. Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, oxapiperazinyl, oxapiperidinyl, tetrahydrofuryl, tetrahydropyranyl, tetrahydrothiophenyl, dihydropyranyl, indolinyl, azepanyl and N-oxides thereof. Attachment of a heterocycloalkyl substituent can occur via either a carbon atom or a heteroatom. A heterocycloalkyl group can be unsubstituted or substituted with one or more suitable groups by one or more aforesaid groups.

As used herein, the term “heteroaryl” alone or in combination with other term(s) means a completely unsaturated ring system containing a total of 5 to 14 ring atoms. At least one of the ring atoms is a heteroatom (i.e., oxygen, nitrogen, or sulfur), with the remaining ring atoms/groups being independently selected from the group consisting of carbon, oxygen, nitrogen or sulfur. A heteroaryl may be a single-ring (monocyclic) or polycyclic ring system.

Examples of “heteroaryl” include but are not limited to pyridyl, indolyl, benzimidazolyl, benzothiazolyl and the like.

As used herein, the term “heterocyclyl” alone or in combination with other term(s) includes both “heterocycloalkyl” and “heteroaryl” groups which are as defined above. Examples of “Heterocyclyl” include, but are not limited to azetidinyl, pyrrolidinyl, piperidinyl, pyridyl, indolyl, benzimidazolyl, benzothiazolyl and the like.

The term “heteroatom” as used herein designates a sulfur, nitrogen or oxygen atom.

As used in the above definitions, the term “optionally substituted” or “substituted” or “optionally substituted with suitable groups” refers to replacement of one or more hydrogen radicals in a given structure with a radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic and aliphatic. It is understood that any substituent may be further substituted.

As used herein, the term ‘compound(s)’ comprises the compounds disclosed in the present disclosure.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “therapeutically effective amount” refers to that amount of the compound being administered sufficient to prevent development of or alleviate to some extent one or more of the symptoms of the condition or disorder being treated.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

As used herein, the phrase “pharmaceutically acceptable excipient” refers to a pharmaceutically acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al, Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, “pharmaceutically acceptable salt(s)” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols or acetonitrile (ACN) are preferred.

The term “stereoisomers” refers to any enantiomers, diastereoisomers, or geometrical isomers of the compounds of Formula (I), (IA), (IB), (IC), (ID), (IE), (IF), (IG), (IH), (IJ), (IK), (IL) and (IM) wherever they are chiral or when they bear one or more double bonds. When the compounds of the formula (I), (IA), (IB), (IC), (ID), (IE), (IF), (IG), (IH), (IJ), (IK), (IL) and (IM) are chiral, they can exist in racemic or in optically active form. It should be understood that the disclosure encompasses all stereochemical isomeric forms, including diastereomeric, enantiomeric and epimeric forms, as well as d-isomers and l-isomers and mixtures thereof.

Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present disclosure may exist as geometric isomers. The present disclosure includes all cis, trans, syn, anti, R and S, entgegen (E) and zusammen (Z) isomers as well as the appropriate mixtures thereof.

The present disclosure provides compounds of formula (I), which are useful for the modulation of CCR4.

The present disclosure further provides pharmaceutical compositions comprising the said compounds of formula (I), and their derivatives as therapeutic agents.

It will be apparent to those skilled in the art that various modifications and variations can be made to the compounds, compositions, and methods described herein without departing from the scope or spirit of various embodiments disclosed herein. For instance, features illustrated or described as part of one embodiment can be applied to another embodiment to yield a still further embodiment. Thus, it is intended that the present application includes such modifications and variations and their equivalents. Other objects, features, and aspects of the present application are disclosed in, or are obvious from, the following detailed description. It is to be understood by one of ordinary skill in the art that the present discussion is a description of exemplary embodiments and is not to be construed as limiting the broader aspects of the present disclosure.

The embodiments below are illustrative of the present disclosure and are not intended to limit the claims to the specific embodiments exemplified.

In a first embodiment, the present application provides compounds of formula (I),

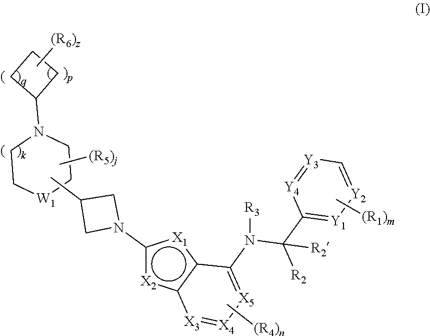

or a pharmaceutically acceptable salt thereof or a stereoisomer thereof;
wherein,
W₁ is C, N or O;
X₁ and X₂ are each independently CH, N, NRₓ, O, S or —S(O)—;
X₃, X₄ and X₅ are each independently C or N;
Rₓ is hydrogen or C₁-C₆ alkyl;
Y₁, Y₂, Y₃, and Y₄ are each independently C or N;
R₁ at each occurrence is independently C₁-C₆ alkyl, C₂-C₆ alkenyl, C₂-C₆ alkynyl, halo, cyano, amino, nitro, —OR₁ₐ, —C(O)R_{1b} or C₃-C₆ cycloalkyl; wherein each of C₁-C₆ alkyl, C₂-C₆ alkenyl, C₂-C₆ alkynyl, and C₃-C₆ cycloalkyl is optionally substituted with one or more substituents independently selected from halo, hydroxy and C₁-C₆ alkoxy;
R₂ and R₂′ are each independently hydrogen, C₁-C₆ alkyl or halo;
R₃ is hydrogen or C₁-C₆ alkyl;
R₄ at each occurrence is independently C₁-C₆ alkyl, C₂-C₆ alkenyl, C₂-C₆ alkynyl, halo, cyano, amino, nitro, —OR₄ₐ, —C(O)R_{4b}, or C₃-C₆ cycloalkyl; wherein each of C₁-C₆ alkyl, C₂-C₆ alkenyl, C₂-C₆ alkynyl, and C₃-C₆ cycloalkyl is optionally substituted with one or more substituents independently selected from one or more halo, hydroxy and C₁-C₆ alkoxy;
R₅ at each occurrence is independently halo, C₁-C₆ alkyl, C₁-C₆ haloalkyl, C₁-C₆ alkoxy or —C(O)R₅ₐ;
R₆ at each occurrence is independently C₁-C₆ alkyl, halo, C₁-C₆ haloalkyl, cyano, —C(O)R₆ₐ, —(C₁-C₃ alkyl)C(O)R₆ₐ or —OR_{6b};
R₁ₐ and R₄ₐ are each independently hydrogen, halo, C₁-C₆ alkyl, C₁-C₆ haloalkyl, C₃-C₆ cycloalkyl or C₃-C₆ heterocycloalkyl;
R_{1b}, R_{4b} and R₅ₐ are each independently hydrogen, C₁-C₆ alkyl, hydroxy, amino, C₁-C₆ alkylamino, C₁-C₆ haloalkyl, C₃-C₆ cycloalkyl or C₃-C₆ heterocycloalkyl;
R₆ₐ is hydrogen, hydroxy, C₁-C₆ alkyl, C₁-C₆ haloalkyl, C₁-C₆ alkoxy, C₁-C₆ haloalkoxy, amino, C₁-C₆ alkylamino or C₃-C₆ cycloalkyl;
R_{6b} is hydrogen, C₁-C₆ alkyl, C₁-C₆ haloalkyl or C₃-C₆ cycloalkyl;
‘m’ is selected from 0 to 5;
‘n’ is selected from 0 to 3;
‘j’ is selected from 0 to 4;
‘k’ is selected from 0 to 2;
‘p’ and ‘q’ are each independently selected from 1 and 2; and
‘z’ is selected from 0 to 4.

In another embodiment, the present application provides compounds of formula (I), or a pharmaceutically acceptable salt thereof or a stereoisomer thereof;

wherein,
W₁ is CH₂, NH or O;
X₁ and X₂ are each independently CH, N, NRₓ, O, S or —S(O)—;
X₃, X₄ and X₅ are each independently C or N;
Rₓ is hydrogen or alkyl;
Y₁, Y₂, Y₃, and Y₄ are each independently C or N;
R₁ at each occurrence is independently C₁-C₆ alkyl, C₂-C₆ alkenyl, C₂-C₆ alkynyl, halo, cyano, amino, nitro, —OR₁ₐ, —C(O)R_{1b} or C₃-C₆ cycloalkyl; wherein each of C₁-C₆ alkyl, C₂-C₆ alkenyl, C₂-C₆ alkynyl, and C₃-C₆ cycloalkyl is optionally substituted with one or more substituents independently selected from halo, hydroxy and C₁-C₆ alkoxy;
R₂ and R₂′ are each independently hydrogen, C₁-C₆ alkyl or halo;
R₃ is hydrogen or C₁-C₆ alkyl;
R₄ at each occurrence is independently C₁-C₆ alkyl, C₂-C₆ alkenyl, C₂-C₆ alkynyl, halo, cyano, amino, nitro, —OR₄a, —C(O)R_{4b}, or C₃-C₆ cycloalkyl; wherein each of C₁-C₆ alkyl, C₂-C₆ alkenyl, C₂-C₆ alkynyl, and C₃-C₆ cycloalkyl is optionally substituted with one or more substituents independently selected from one or more halo, hydroxy and C₁-C₆ alkoxy;
R₅ at each occurrence is independently halo, C₁-C₆ alkyl, C₁-C₆ haloalkyl, C₁-C₆ alkoxy or —C(O)R₅ₐ;
R₆ at each occurrence is independently C₁-C₆ alkyl, halo, C₁-C₆ haloalkyl, cyano, —C(O)R₆ₐ, —(C₁-C₃ alkyl)C(O)R₆ₐ or —OR_{6b};
R₁ₐ and R₄ₐ are each independently hydrogen, halo, C₁-C₆ alkyl, C₁-C₆ haloalkyl, C₃-C₆ cycloalkyl or C₃-C₆ heterocycloalkyl;
R_{1b}, R_{4b} and R₅ₐ are each independently hydrogen, C₁-C₆ alkyl, hydroxy, amino, C₁-C₆ alkylamino, C₁-C₆ haloalkyl, C₃-C₆ cycloalkyl or C₃-C₆ heterocycloalkyl;
R₆ₐ is hydrogen, hydroxy, C₁-C₆ alkyl, C₁-C₆ haloalkyl, C₁-C₆ alkoxy, C₁-C₆ haloalkoxy, amino, C₁-C₆ alkylamino or C₃-C₆ cycloalkyl;
R_{6b} is hydrogen, C₁-C₆ alkyl, C₁-C₆ haloalkyl or C₃-C₆ cycloalkyl;
‘m’ is selected from 0 to 5;
‘n’ is selected from 0 to 3;
‘j’ is selected from 0 to 4;
‘k’ is selected from 0 to 2;
‘p’ and ‘q’ are each independently selected from 1 and 2; and
‘z’ is selected from 0 to 4.

In another embodiment of the present application, it provides compounds of formula (IA),

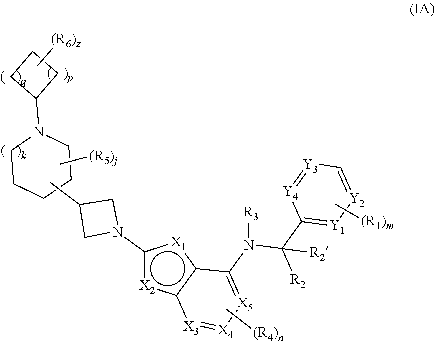

or a pharmaceutically acceptable salt thereof or a stereoisomer thereof.

In another embodiment of the present application, it provides compounds of formula (IB),

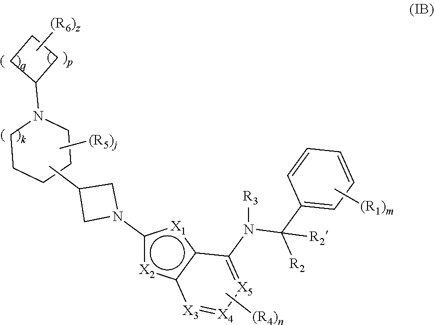

or a pharmaceutically acceptable salt thereof or a stereoisomer thereof.

In another embodiment of the present application, it provides compounds of formulae (IC), (ID), (IE), (IF), (IG), (IH), (IJ), (IK) and (IL):

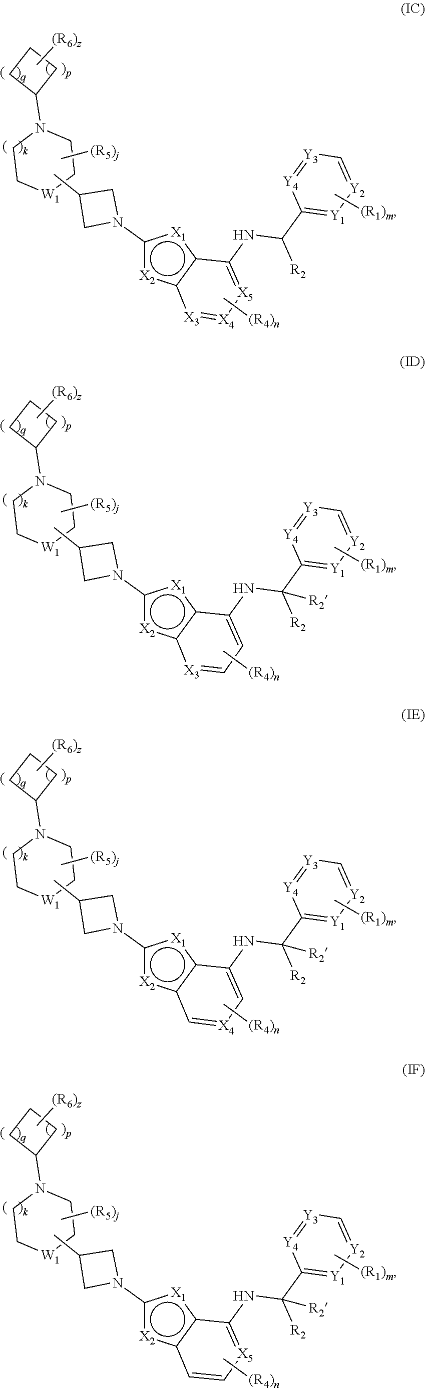

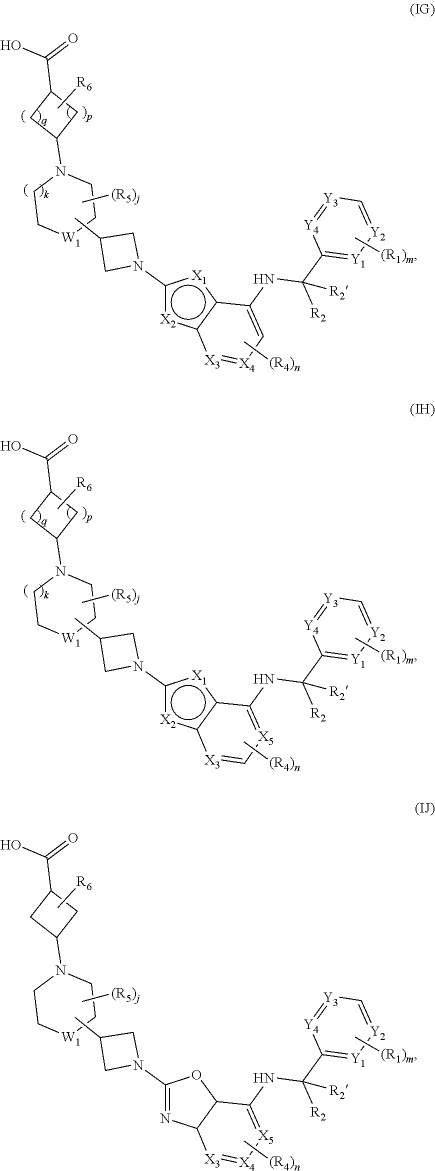

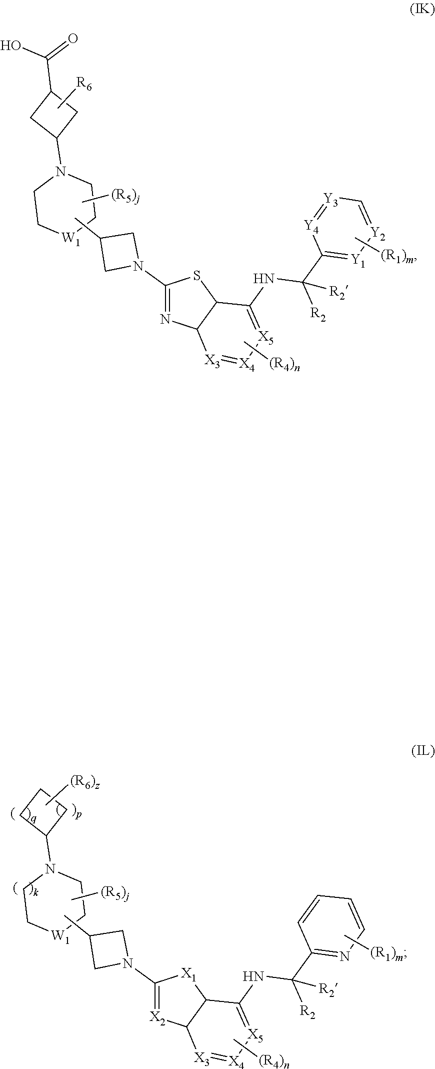

or a pharmaceutically acceptable salt thereof or a stereoisomer thereof.

In another embodiment of the present application, it provides compounds of formulae (IA-1), (IB-1), (IC-1), (ID-1), (IE-1), (IF-1) and (IG-1).

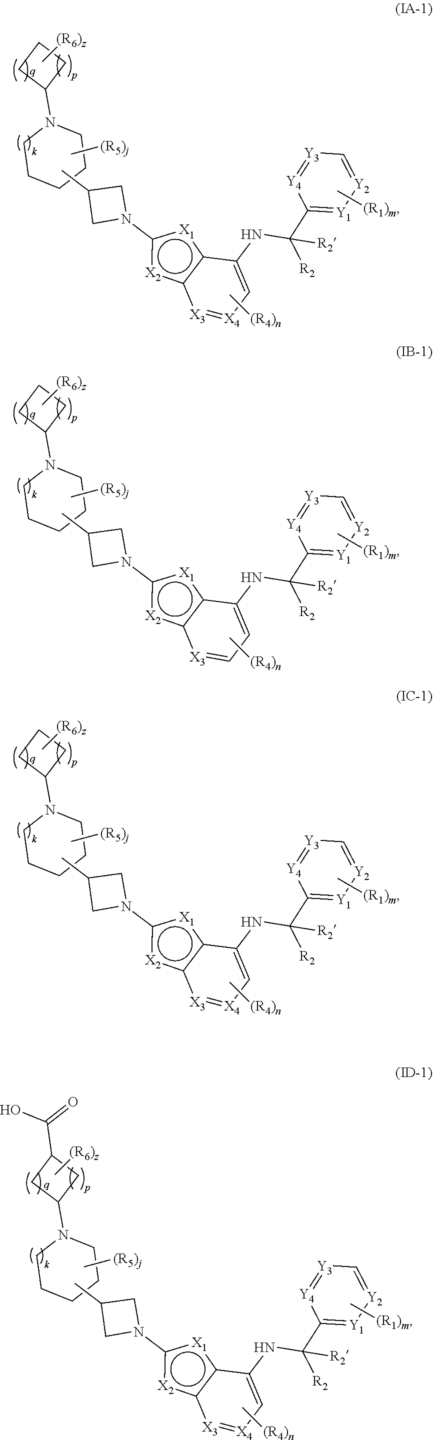

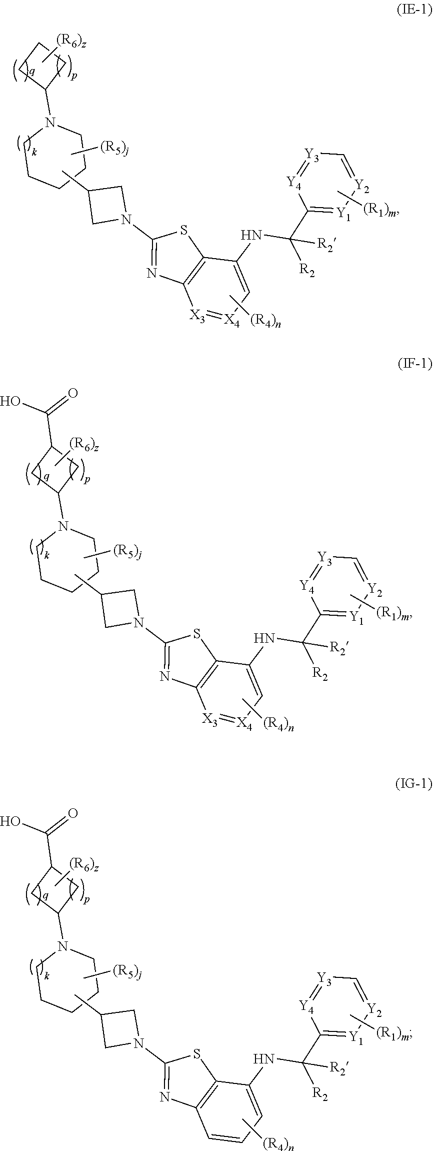

or a pharmaceutically acceptable salt thereof or a stereoisomer thereof.

In another embodiment of the present application, it provides compounds of formulae (IA-2), (IB-2), (IC-2), (ID-2), (IE-2), (IF-2), (IG-2) and (IH-2):

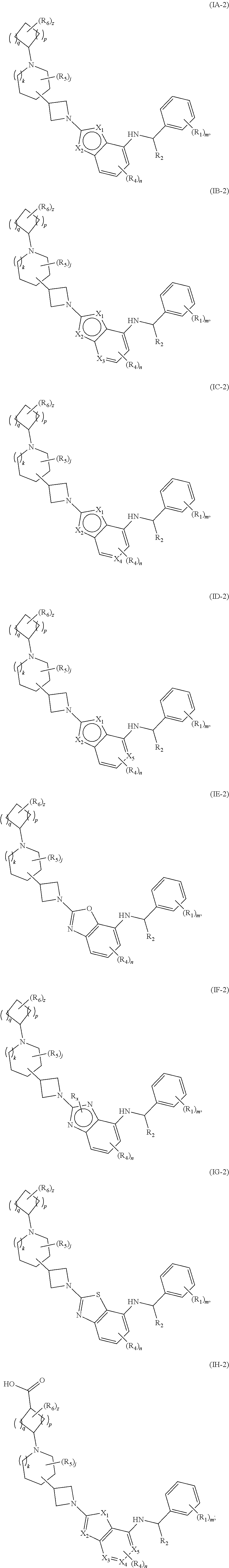

or a pharmaceutically acceptable salt thereof or a stereoisomer thereof.

In one embodiment of the present application, it provides compounds of formula (I) to (IL), (IA-1) to (IG-1) and (IA-2) to (IH-2), wherein the ring represented by

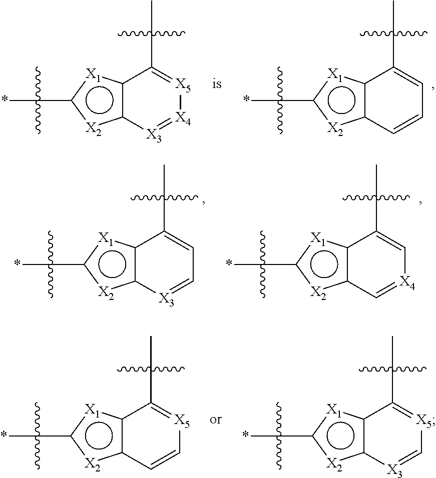

wherein the asterisk mark represents the point of attachment with azetidine ring.

In one embodiment of the present application, it provides compounds of formula (I) to (IL), (IA-1) to (IG-1) and (IA-2) to (IH-2), wherein the ring represented by

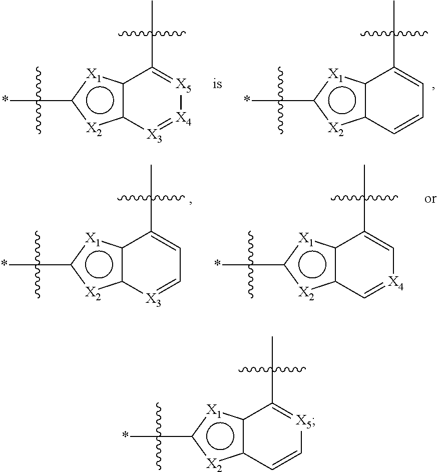

wherein the asterisk mark represents the point of attachment with azetidine ring.

In one embodiment, the ring represented by,

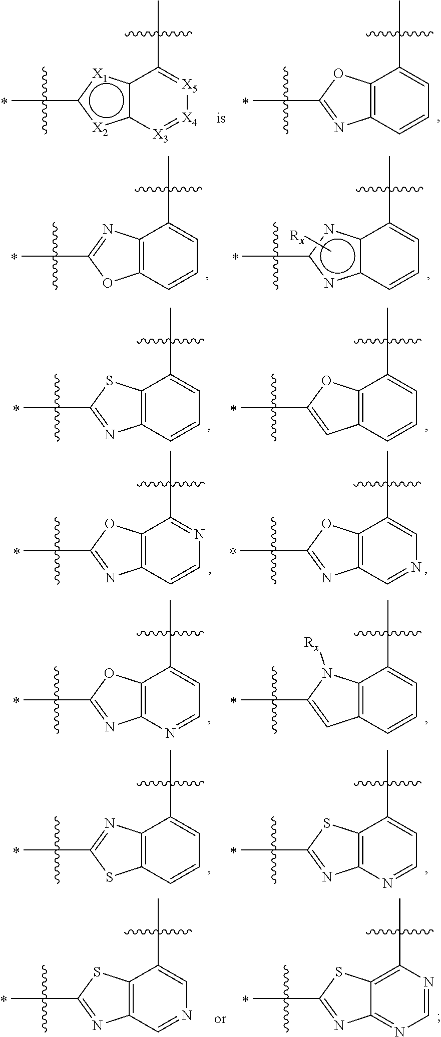

wherein the asterisk mark represents the point of attachment with azetidine ring.

In one embodiment, the ring represented by,

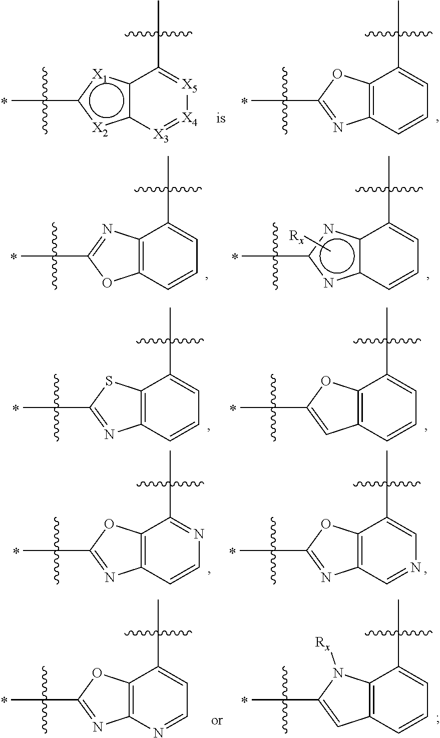

wherein the asterisk mark represents the point of attachment with azetidine ring.

In one embodiment, the ring represented by,

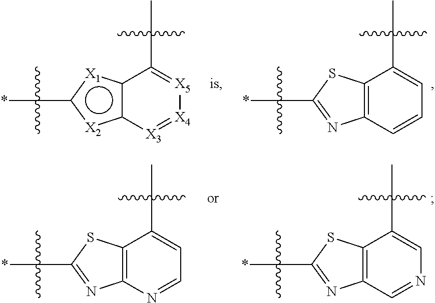

wherein the asterisk mark represents the point of attachment with azetidine ring.

In one embodiment, the ring represented by,

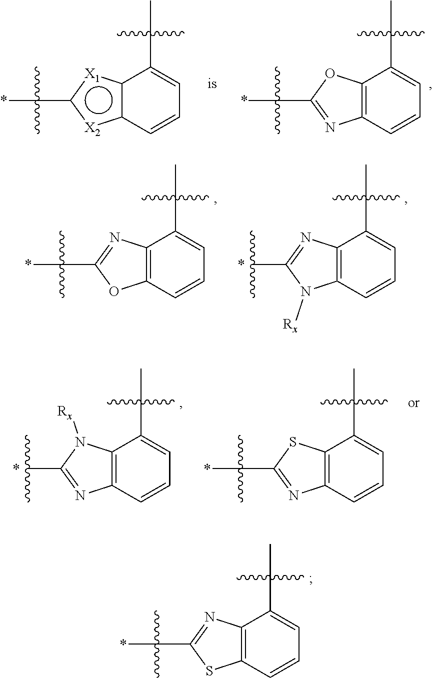

wherein the asterisk mark represents the point of attachment with azetidine ring.

In one embodiment, the ring represented by,

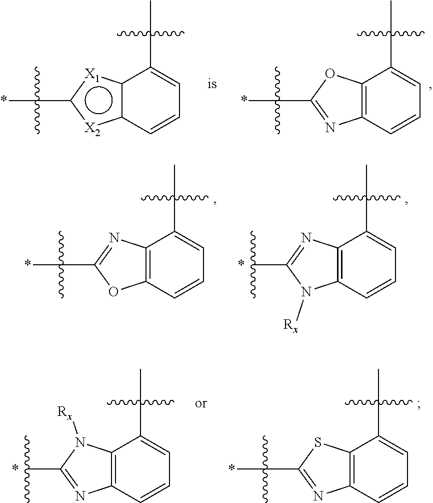

wherein the asterisk mark represents the point of attachment with azetidine ring.

In one embodiment, the ring represented by,

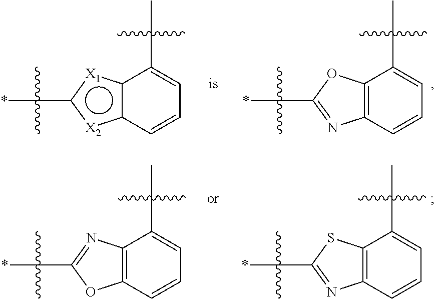

wherein the asterisk mark represents the point of attachment with azetidine ring.

In one embodiment of the present application, wherein the ring

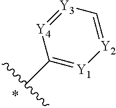

is independently

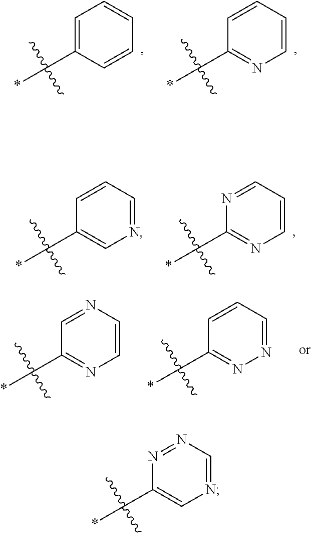

wherein asterisk mark represents point of attachment with carbon atom having R₂ and R₂′ groups.

In one embodiment X₁ is N, NRₓ, O, S or —S(O)—.

In one embodiment X₂ is CH, N, O, S or NRₓ.

In one embodiment X₁ is N, NRₓ, O, S or —S(O)— and X₂ is N.

In one embodiment X₂ is CH, N, NRₓ, O, or S or —S(O)— and X₁ is N.

In one embodiment, X₁ is N, O or S; and X₂ is N.

In one embodiment, X₁ is N, O, S or —S(O)—.

In one embodiment, X₁ is N, O or S.

In one embodiment, X₁ is S or —S(O)—.

In one embodiment, X₁ is S.

In one embodiment, X₂ is CH, N, O or NRₓ.

In one embodiment, X₂ is CH, N or O.

In one embodiment, X₂ is N, O or NRₓ.

In one embodiment, X₂ is N or O.

In one embodiment, X₂ is N.

In one embodiment, X₁ is S and X₂ is N.

In one embodiment, X₁ is N, NRₓ, O or S.

In one embodiment, X₁ is N, O or S.

In one embodiment, X₁ is N or O.

In one embodiment, X₁ is O.

In one embodiment, X₂ is C, N or O.

In one embodiment, X₁ is O and X₂ is N.

In one embodiment R₁ at each occurrence is halo.

In one embodiment R₁ at each occurrence is halo and ‘m’ is 0 to 3.

In one embodiment R₁ at each occurrence is halo and ‘m’ is 2.

In one embodiment, R₂ is C₁-C₆ alkyl or halo and R₂′ is hydrogen.

In one embodiment, R₂ is C₁-C₆ alkyl and R₂′ is hydrogen.

In one embodiment, R₃ is hydrogen.

In one embodiment, R₄ at each occurrence is independently halo, C₁-C₆ alkyl, cyano, —OR₄ₐ or —C(O)R_{4b}; wherein R₄ₐ is hydrogen, C₁-C₆ alkyl, C₁-C₆ haloalkyl and R_{4b} is hydrogen, C₁-C₆ alkyl, amino, C₁-C₆ alkylamino, or C₁-C₆ haloalkyl.

In one embodiment, R₄ at each occurrence is independently halo, C₁-C₆ alkyl, cyano and —OR₄ₐ; wherein R₄ₐ is hydrogen, halo, C₁-C₆ alkyl or C₁-C₆ haloalkyl.

In one embodiment, R₄ at each occurrence is independently halo, C₁-C₆ alkyl or —OR₄a.

In one embodiment, R₄ at each occurrence is independently halo, C₁-C₆ alkyl or C₁-C₆ haloalkyl.

In one embodiment, R₄ at each occurrence is independently halo or C₁-C₆ haloalkyl.

In one embodiment, R₄ at each occurrence is selected from halo, C₁-C₆ alkyl and —OR₄ₐ; wherein R₄ₐ is hydrogen, halo, C₁-C₆ alkyl or C₁-C₆ haloalkyl.

In one embodiment, R₆ at each occurrence is independently C₁-C₆ alkyl, —(C₁-C₃ alkyl)C(O)R₆ₐ or —C(O)R₆ₐ; wherein R₆ₐ is hydroxy.

In one embodiment, R₆ at each occurrence is independently C₁-C₆ alkyl or —C(O)R₆ₐ; wherein R₆ₐ is hydroxy.

In one embodiment, each R₆ at each occurrence is independently selected from —CH₃, —CH₂C(O)OH and —C(O)OH.

In one embodiment, each R₆ at each occurrence is independently selected from —CH₃ and —C(O)OH.

In one embodiment, W₁ is C, N or O.

In one embodiment, X₁ and X₂ are each independently N, NRₓ, O, S or —S(O)—;
X₃, X₄ and X₅ are each independently C or N;
Rₓ is C₁-C₆ alkyl;
Y₁, Y₂, Y₃, and Y₄ are each independently C or N;
R₁ at each occurrence is independently halo;
R₂ and R₂′ are each independently hydrogen, C₁-C₆ alkyl or halo;
R₃ is hydrogen;
R₄ at each occurrence is independently C₁-C₆ alkyl, cyano, halo, —OR₄ₐ or C(O)R_{4b}; wherein C₁-C₆ alkyl is optionally independently substituted with one or more substituents selected from one or more halo and hydroxy;
R₄ₐ is hydrogen, halo, C₁-C₆ alkyl or C₁-C₆ haloalkyl;
R_{4b} is hydrogen, C₁-C₆ alkyl, hydroxy, amino, C₁-C₆ alkylamino;
R₆ at each occurrence is independently C₁-C₆ alkyl, or —C(O)R₆ₐ;
R₆ₐ is hydroxy;
‘m’ is selected from 0 to 3;
‘n’ is selected from 0 to 2;
‘j’ is 0 to 2;
‘k’ is 1;
‘p’ and ‘q’ are each 1; and
‘z’ is selected from 0 to 4.

In one embodiment, X₁ and X₂ are each independently N, NRₓ, O, S or —S(O)—;
X₃, X₄ and X₅ are each independently C or N;
Rₓ is C₁-C₆ alkyl;
Y₁, Y₂, Y₃, and Y₄ are each independently C or N;
R₁ at each occurrence is independently halo;
R₂ and R₂′ are each independently hydrogen, C₁-C₆ alkyl or halo;
R₃ is hydrogen;
R₄ at each occurrence is independently C₁-C₆ alkyl, halo or —OR₄ₐ; wherein R₄ₐ is hydrogen, halo, C₁-C₆ alkyl or C₁-C₆ haloalkyl;
R₆ at each occurrence is independently C₁-C₆ alkyl or —C(O)R₆ₐ;
R₆ₐ is hydroxy;
‘m’ is selected from 0 to 3;
‘n’ is selected from 0 to 2;
‘j’ is 0 to 2;
‘k’ is 1;
‘p’ and ‘q’ are each 1; and
‘z’ is selected from 0 to 4.

In one embodiment, X₁ and X₂ are each independently N, NRₓ, O or S;
X₃, X₄ and X₅ are each independently C or N;
Rₓ is C₁-C₆ alkyl;
R₁ at each occurrence is independently halo;
R₂ and R₂′ are each independently hydrogen or C₁-C₆ alkyl;
R₃ is hydrogen;
R₄ at each occurrence is independently C₁-C₆ alkyl, halo or —OR₄ₐ; wherein the C₁-C₆ alkyl is optionally independently substituted with one or more halo;
R₄ₐ is C₁-C₆ alkyl or C₁-C₆ haloalkyl;
R₆ at each occurrence is independently C₁-C₆ alkyl or —C(O)R₆ₐ;
R₆ₐ is hydroxy;
‘m’ is selected from 0 to 3;
‘n’ is selected from 0 to 2;
‘j’ is 0;
‘k’ is 1;
‘p’ and ‘q’ are each 1; and
‘z’ is selected from 0 to 4.

In one embodiment, j is 0.

In one embodiment, k is 1.

In one embodiment, m is 1 to 3.

In one embodiment, n is 0 to 2.

In one embodiment, p and q are each 1.

In certain embodiments, the present application provides a compound selected from:

| Comp. | |
|---|---|
| No. | Structure |
| 1 | 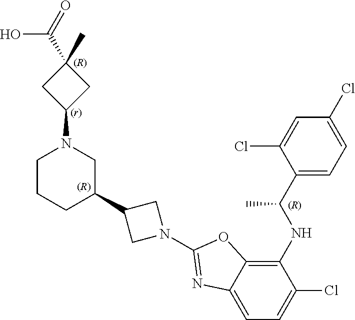 |
| 2 | 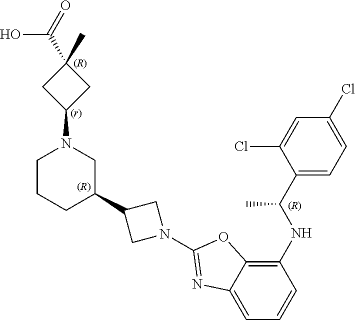 |
| 3 | 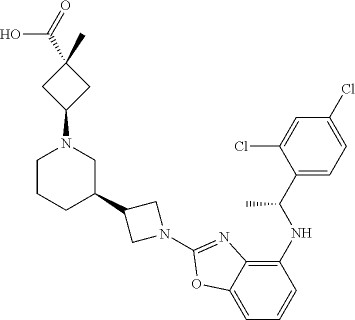 [SMILES: C[C@@H](Nc1cccc2oc(N3CC([C@H]4CCCN([C@H]5C[C@](C)(C(=O)O)C5)C4)C3)nc12)c1ccc(Cl)cc1Cl] |
| 4 | 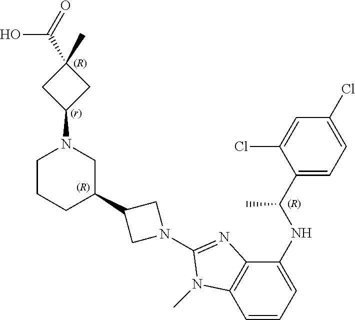 |
| 5 | 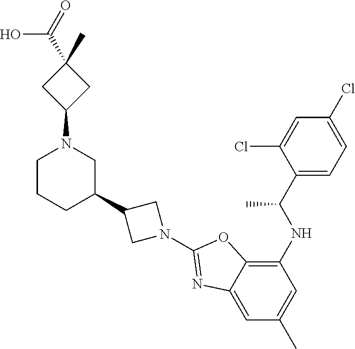 [SMILES: Cc1cc(N[C@H](C)c2ccc(Cl)cc2Cl)c2oc(N3CC([C@H]4CCCN([C@H]5C[C@](C)(C(=O)O)C5)C4)C3)nc2c1] |
| 6 | 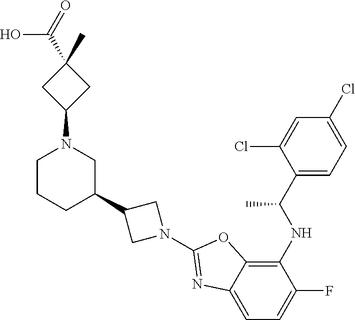 [SMILES: C[C@@H](Nc1c(F)ccc2nc(N3CC([C@H]4CCCN([C@H]5C[C@](C)(C(=O)O)C5)C4)C3)oc12)c1ccc(Cl)cc1Cl] |
| 7 | 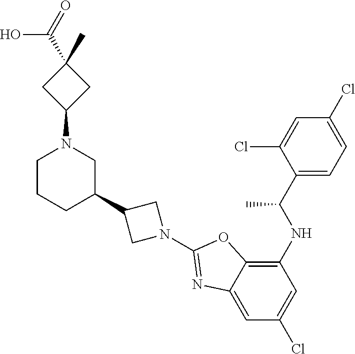 [SMILES: C[C@@H](Nc1cc(Cl)cc2nc(N3CC([C@H]4CCCN([C@H]5C[C@](C)(C(=O)O)C5)C4)C3)oc12)c1ccc(Cl)cc1Cl] |
| 8 | 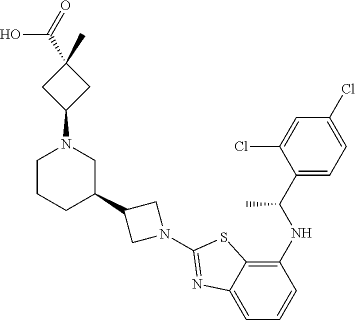 [SMILES: C[C@@H](Nc1cccc2nc(N3CC([C@H]4CCCN([C@H]5C[C@](C)(C(=O)O)C5)C4)C3)sc12)c1ccc(Cl)cc1Cl] |
| 9 | 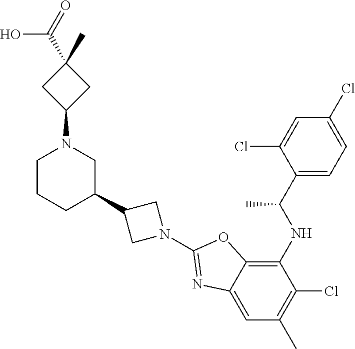 [SMILES: Cc1cc2nc(N3CC([C@H]4CCCN([C@H]5C[C@](C)(C(=O)O)C5)C4)C3)oc2c(N[C@H](C)c2ccc(Cl)cc2Cl)c1Cl] |
| 10 | 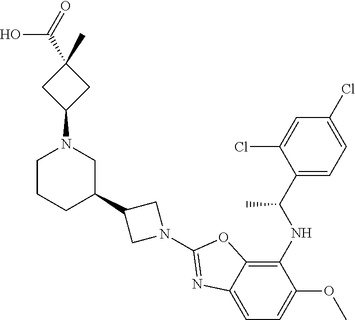 [SMILES: COc1ccc2nc(N3CC([C@H]4CCCN([C@H]5C[C@](C)(C(=O)O)C5)C4)C3)oc2c1N[C@H](C)c1ccc(Cl)cc1Cl] |
| 11 | 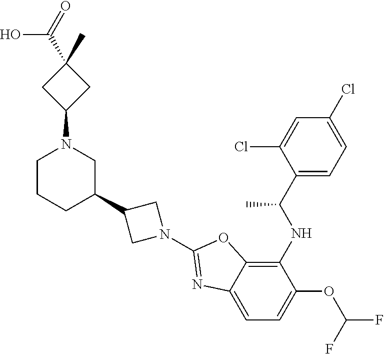 [SMILES: C[C@@H](Nc1c(OC(F)F)ccc2nc(N3CC([C@H]4CCCN([C@H]5C[C@](C)(C(=O)O)C5)C4)C3)oc12)c1ccc(Cl)cc1Cl] |
| 12 | 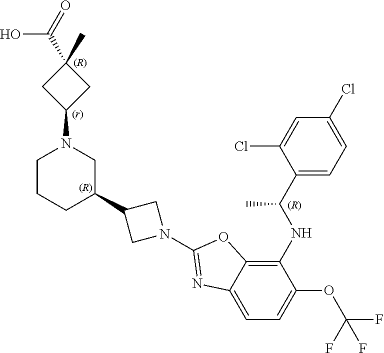 |
| 13 | 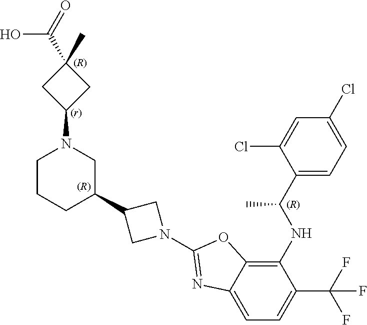 |
| 14 | 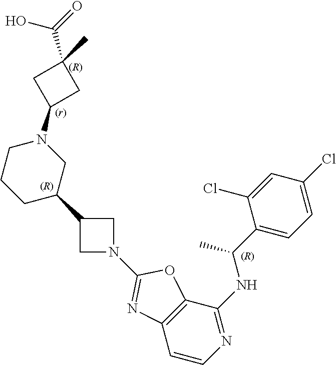 |
| 15 | 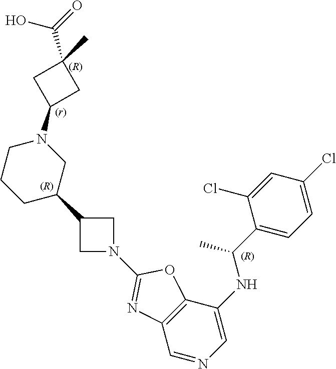 |
| 16 | 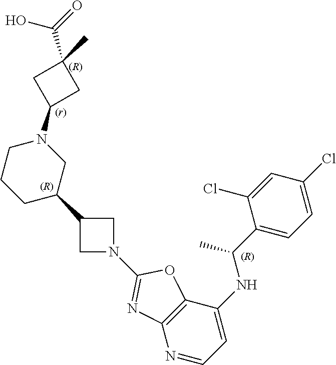 |
| 17 | 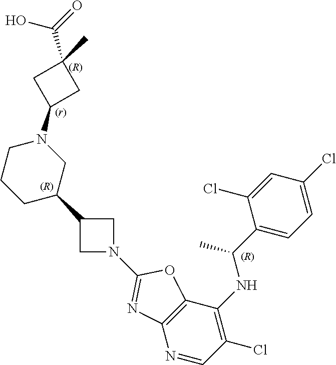 |
| 18 | 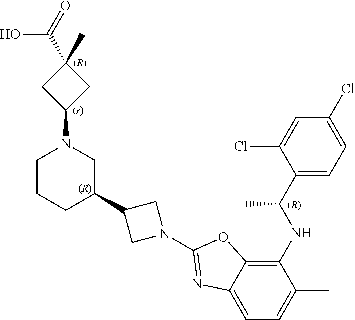 |
| 19 | 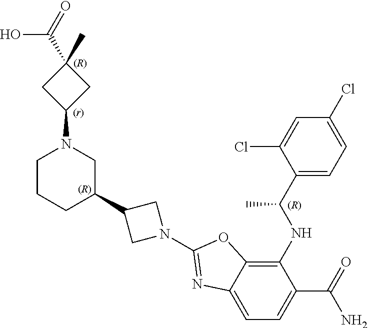 |
| 20 | 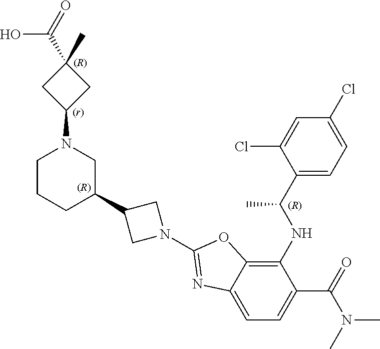 |
| 21 | 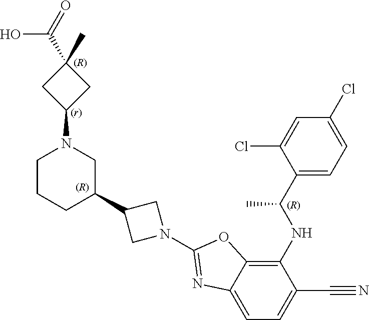 |
| 22 | 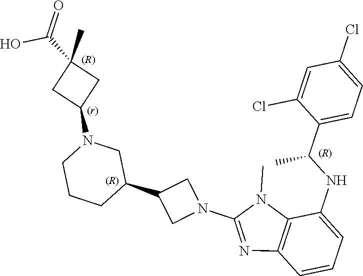 |
| 23 | 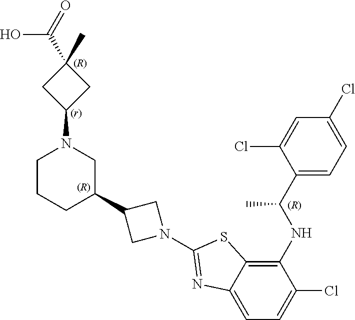 |
| 24 | 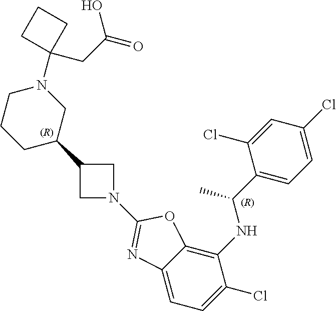 |
| 25 | 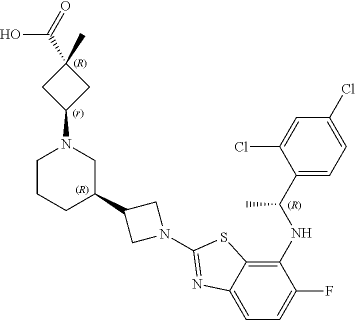 |
| 26 | 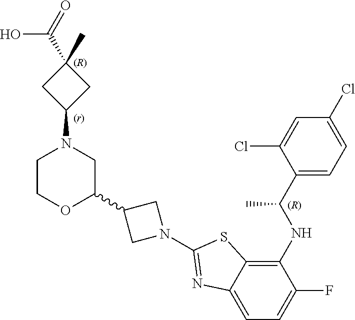 |
| 27 | 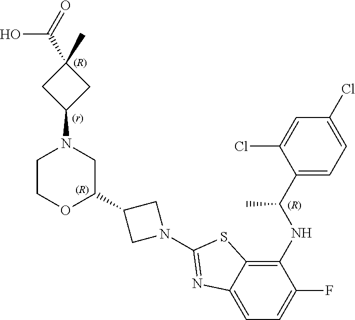 |
| 28 | 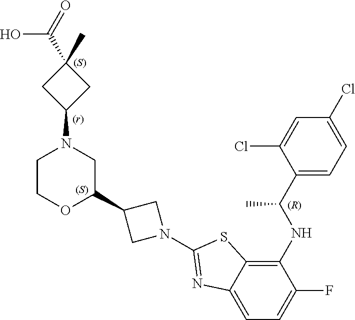 |
| 29 | 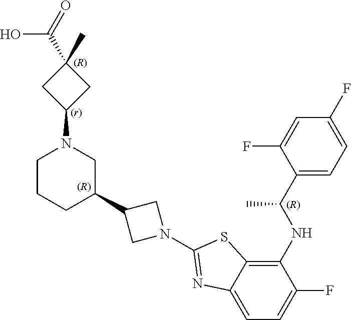 |
| 30 | 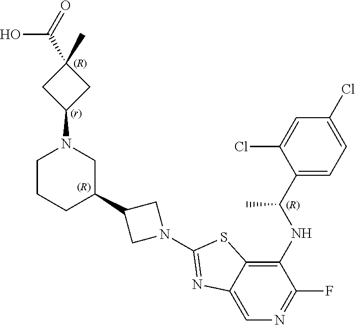 |
| 31 | 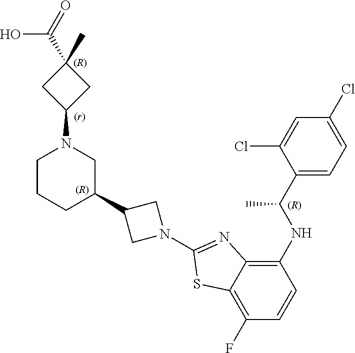 |
| 32 | 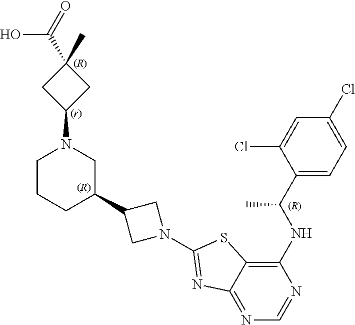 |
| 33 | 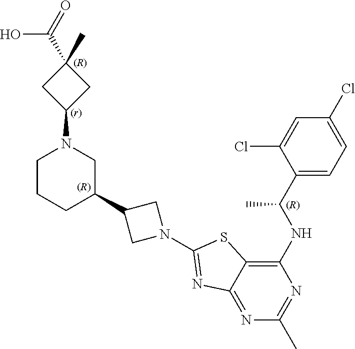 |
| 34 | 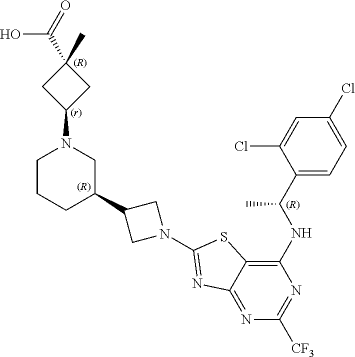 |
| 35 | 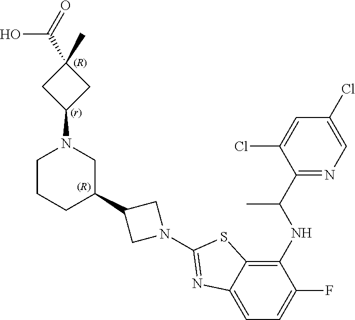 |
| 36 | 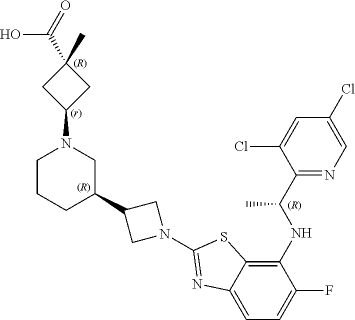 |
| | Isomer-1 of compound 35 |
| 37 | 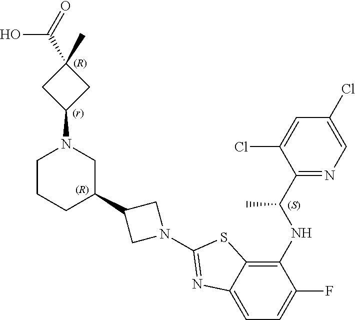 |
| | Isomer-2 of compound 35 |
| 38 | 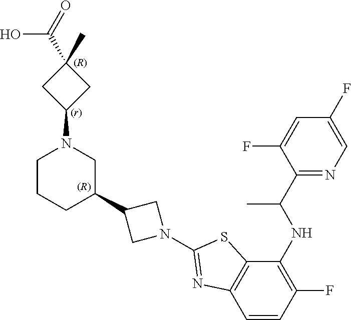 |
| 39 | 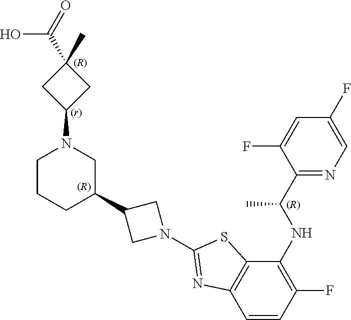 |
| | Isomer-1 of compound 38 |
| 40 | 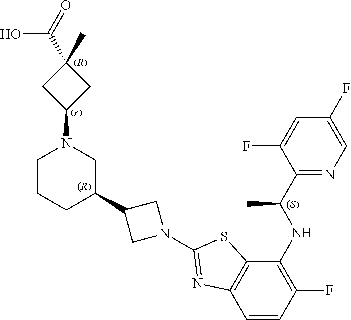 |
| | Isomer-2 of compound 38 |
| 41 | 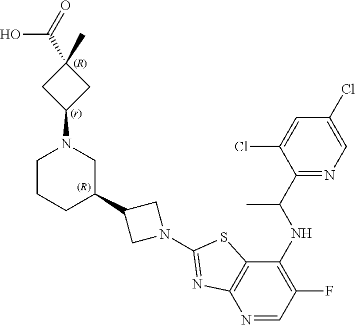 |
| 42 | 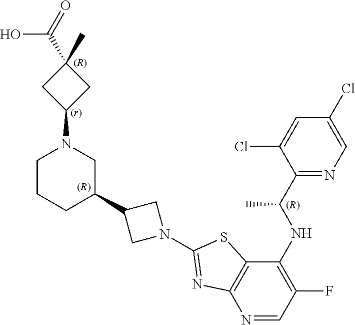 |
| | Isomer-1 of compound 41 |
| 43 | 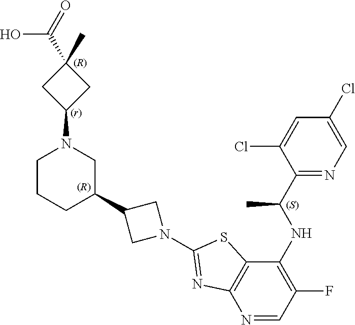 |
| | Isomer-2 of compound 41 |
| 44 | 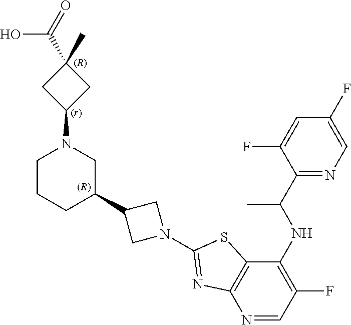 |
| 45 | 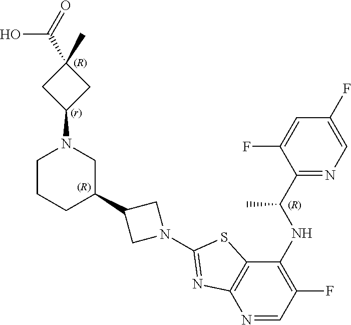 |
| | Isomer-1 of compound 44 |
| 46 | 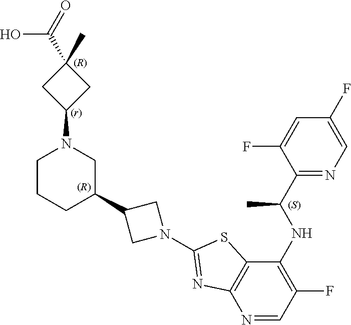 |
| | Isomer-2 of compound 44 |
| 47 | 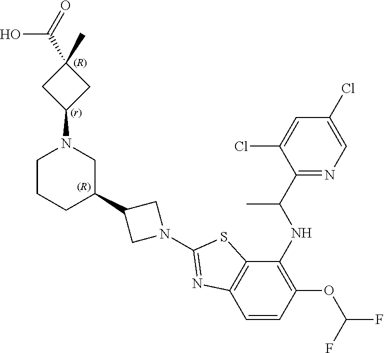 |
| 48 | 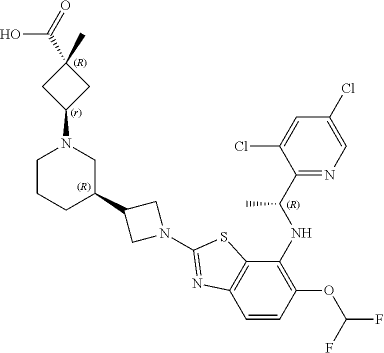 |
| | Isomer-1 of compound 47 |
| 49 | 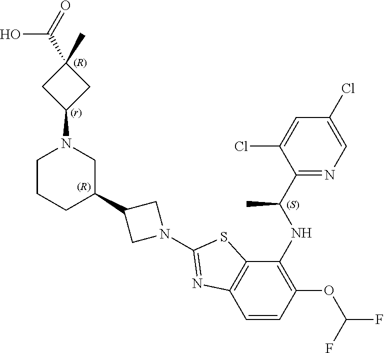 |
| | Isomer-2 of compound 47 |
| 50 | 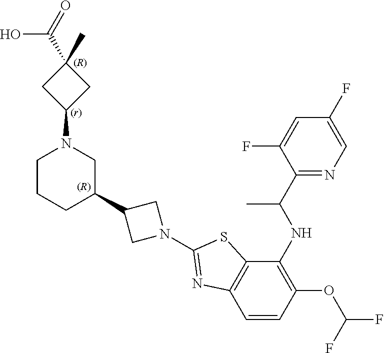 |
| 51 | 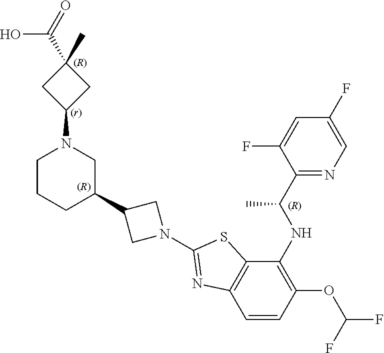 |
| | Isomer-1 of compound 50 |
| 52 | 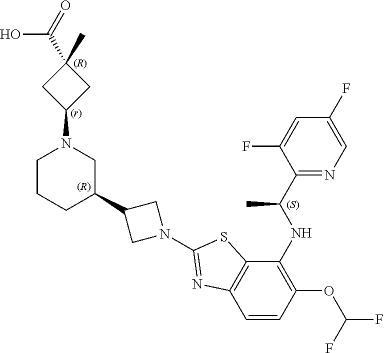 |
| | Isomer-2 of compound 50 |
| 53 | 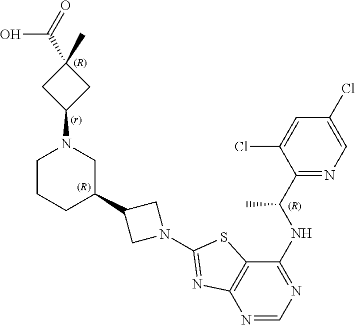 |
| 54 | 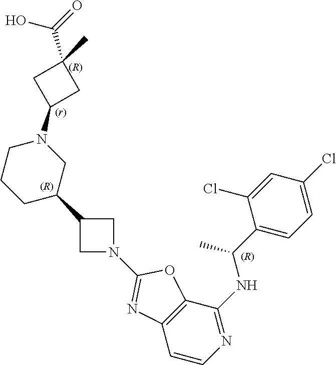 |
| 55 | 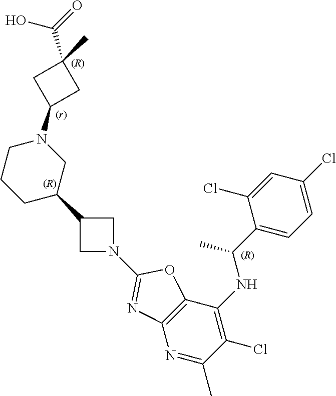 |
| 56 | 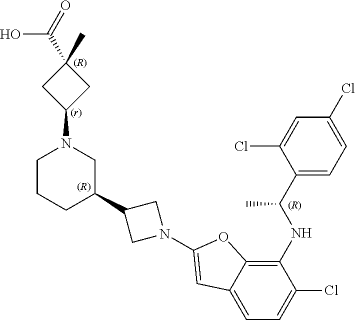 |
| 57 | 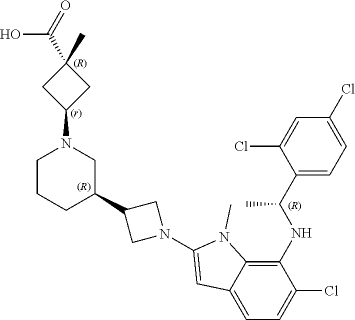 |
| 58 | 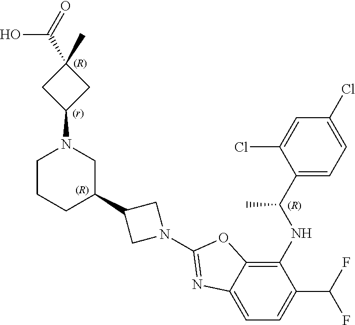 |
| 59 | 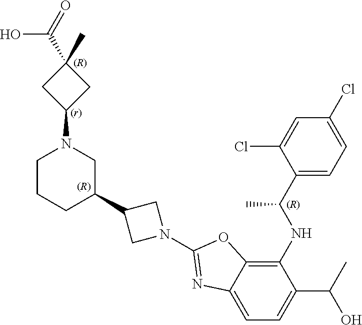 |
| 60 | 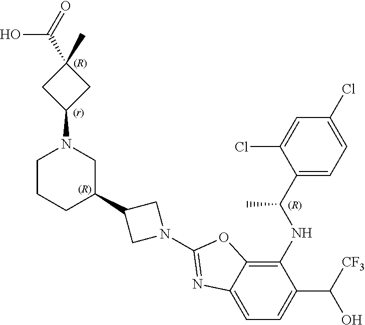 |
| 61 | 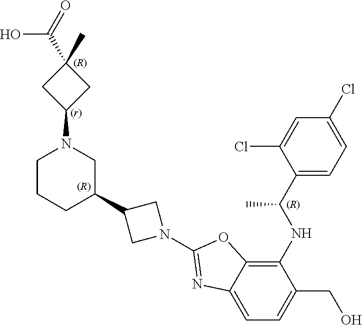 |
| 62 | 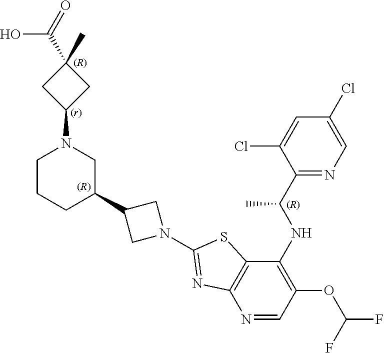 |
| 63 | 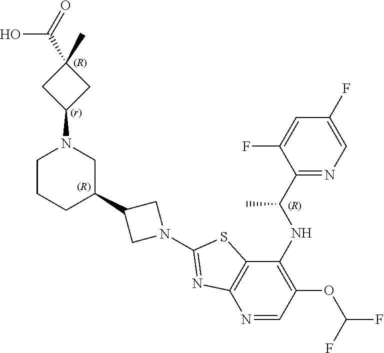 |
| 64 | 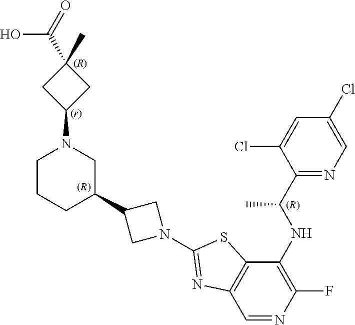 |
| 65 | 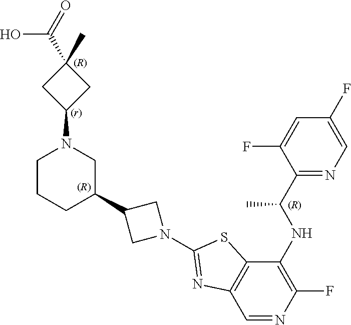 |
| 66 | 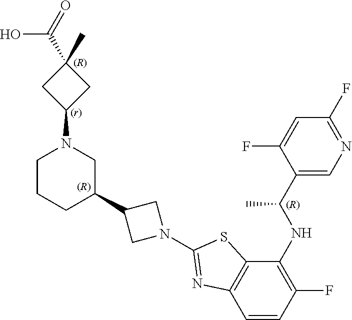 |
| 67 | 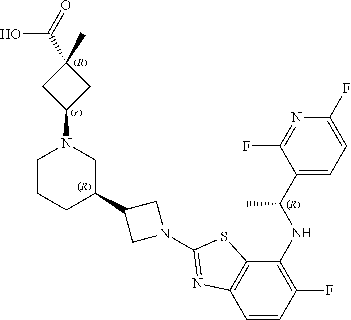 |
| 68 | 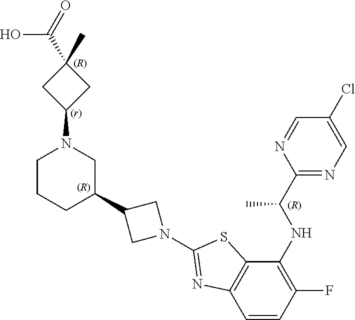 |
| 69 | 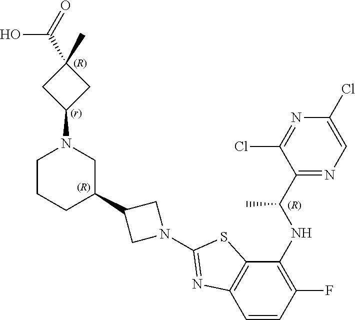 |
| 70 | 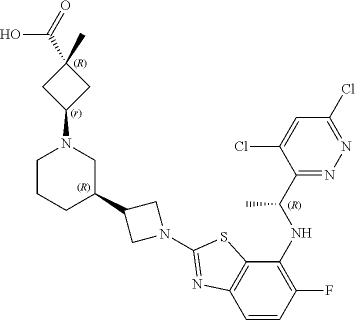 |
| 71 | 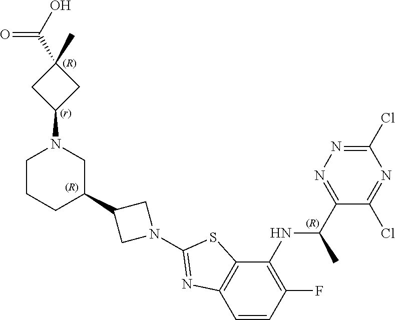 |
| 72 | 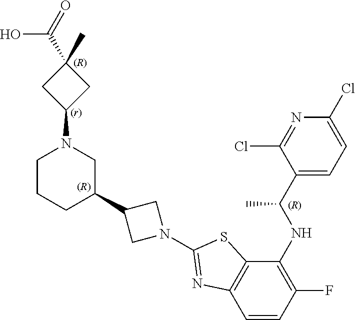 |
| 73 | 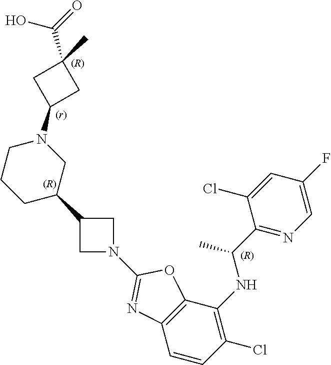 |
| 74 | 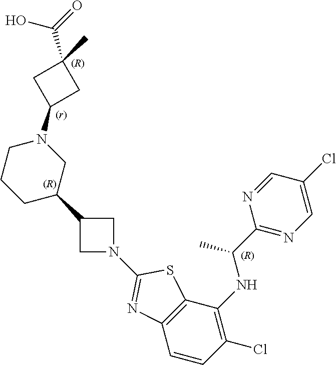 |
| 75 | 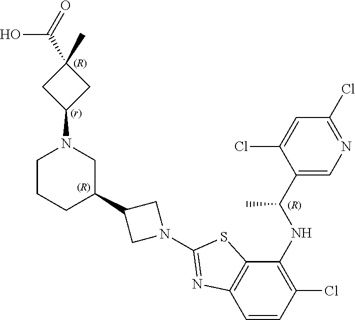 |

or a pharmaceutically acceptable salt thereof or a stereoisomer thereof.

In certain embodiments, the present disclosure provides a pharmaceutical composition comprising a compound of formula (I), or a pharmaceutically acceptable salt thereof or a stereoisomer thereof as described herein and at least one Pharmaceut acceptable excipient (such as a pharmaceutically acceptable carrier or dilutent). Preferably, the pharmaceutical composition comprises a therapeutically effective amount of at least one compound described herein. The compounds described in the present disclosure may be associated with a pharmaceutically acceptable excipient (such as a carrier or a dilutent) or be diluted by a carrier or enclosed within a carrier which can be in the form of a capsule, sachet, paper or other container.

In yet another embodiment, the compounds of the present disclosure are CCR4 modulators.

In yet another embodiment, the compound of formula (I) is a CCR4 modulator.

In another embodiment, the present disclosure provides pharmaceutical composition for use in treating and/or preventing a disease and/or disorder responsive to the modulation of CCR4 activity.

In another embodiment, the present disclosure provides pharmaceutical composition comprising a compound of formula (I), or a pharmaceutically acceptable salt or a stereoisomer thereof, for use in the manufacture of a medicament for treating and/or preventing a disease and/or disorder responsive to the modulation of CCR4 activity.

In another embodiment, the present disclosure provides a pharmaceutical composition comprising a compound of formula (I), or a pharmaceutically acceptable salt or a stereoisomer thereof and at least one pharmaceutically acceptable carrier or excipient.

In an embodiment, the present disclosure provides pharmaceutical composition comprising the compound of formula (I), for use in treating a subject suffering from a disease or condition associated with CCR4.

In an embodiment, the present disclosure provides pharmaceutical composition comprising the compound of formula (I), for use in the manufacture of a medicament for treating a subject suffering from a disease or condition associated with CCR4.

The compounds of the disclosure are typically administered in the form of a pharmaceutical composition. Such compositions can be prepared using procedures well known in the pharmaceutical art and comprise at least one compound of the present disclosure. The pharmaceutical composition of the present disclosure comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, solvents and the like.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present disclosure may be formulated to provide desired release profile.

Administration of the compounds of the disclosure, in pure form or in an appropriate pharmaceutical composition, can be carried out using any of the accepted routes of administration of pharmaceutical compositions. The route of administration may be any route which effectively transports the active compound of the present disclosure to the appropriate or desired site of action. Suitable routes of administration include, but are not limited to, oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present disclosure may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms and suitable pharmaceutical excipients can also be well used and adjusted by those skilled in the art. All changes and modifications are envisioned within the scope of the present disclosure.

In one embodiment, the compounds as disclosed in the present disclosure are formulated for pharmaceutical administration. Yet another embodiment of the present disclosure provides use of the compounds as disclosed in the present application in the treatment and prevention of diseases and/or disorder responsive to the modulation of CCR4 activity.

Yet another embodiment of the present disclosure provides use of the compound or a pharmaceutically acceptable salt thereof, in treating and/or preventing a disease for which the symptoms thereof are treated, improved, diminished and/or prevented by modulation of CCR4.

According to yet another embodiment, the CCR4 mediated disorder and/or disease or condition is cancer or an inflammatory disease or disorder.

According to yet another embodiment, the CCR4 mediated disorder and/or disease or condition is cancer.

In yet another aspect of an embodiment, the cancer is colon cancer, pancreatic cancer, intestinal cancer, breast cancer, lung cancer, gastric cancer, liver cancer or colorectal cancer.

In yet another aspect of an embodiment, the cancer is colon cancer, pancreatic cancer or intestinal cancer.

According to yet another embodiment, the CCR4 mediated disorder and/or disease or condition is an inflammatory disease or disorder.

In yet another aspect of an embodiment, the inflammatory disease or disorder is dermatitis.

In yet another aspect of an embodiment, the inflammatory disease or disorder is atopic dermatitis or contact dermatitis.

In yet another aspect of an embodiment, the inflammatory disease or disorder is atopic dermatitis.

In yet another aspect of an embodiment, the inflammatory disease or disorder is contact dermatitis.

In yet another aspect of an embodiment, the inflammatory disease or disorder is prurigo nodularis.

In yet another aspect of an embodiment, the inflammatory disease or disorder is related to but not limited to Th2-associated inflammation in various mucosal barriers including lung and upper respiratory tissues. Accordingly, the present disclosure provides compounds for the use in respiratory allergies or for asthma or for chronic rhinosinusitis with or without nasal polyposis (CRSwNP).

In yet another aspect of an embodiment, the inflammatory disease or disorder is related to but not limited to Th2-associated inflammation in various mucosal barriers including upper and lower alimentary tract tissues. Accordingly, the present disclosure provides compounds for the use in eosinophilic esophagitis (EoE) as well as but not limited to inflammatory bowel disease (IBD). In yet another embodiment, the present disclosure provides compounds for treatment of ulcerative colitis (UC) including but not limited to Th2-associated UC.

According to yet another embodiment, the present disclosure provides compound of formula (I) for use in the treatment of cancer.

According to yet another embodiment, the subject is a mammal including human.

According to yet another embodiment, the present disclosure provides compounds or pharmaceutically acceptable salts or stereoisomers thereof, for use as a medicament.

According to yet another embodiment, the disclosure provides the use of the compounds of the present disclosure in the manufacture of a medicament.

According to yet another embodiment, the present disclosure provides compounds or pharmaceutically acceptable salts or stereoisomers thereof, for use in the treatment of cancer or an inflammatory disease or disorder. In yet another aspect of an embodiment, the cancer is colon cancer, pancreatic cancer, intestinal cancer, breast cancer, lung cancer, gastric cancer, liver cancer or colorectal cancer. In yet another aspect of an embodiment, the cancer is skin cancer including but not limited to melanoma or invasive adenocarcinoma.

In yet another embodiment, the present disclosure provides compounds for use in the treatment of haematological cancers including but not limited to T cell lymphomas and leukaemia. The present disclosure further provides compounds for use in established HTLV-1 viral induced T cell leukaemia and lymphoma or for the prevention of leukaemogenesis in HTLV-1 positive patients.

In yet another embodiment, the present disclosure provides compounds for use in the treatment and/or prevention of CCR4 activity that promotes metastasis due to migration of tumor cells expressing CCR4 to remote target organs that express CCL17 or CCL22 through lymphatics or blood circulation. Accordingly, the present disclosure provides compounds for use to inhibit metastasis to secondary lymphoid structures including but not limited to draining lymph nodes, downstream lymph nodes, thymus, lung, liver, bone marrow, skin, intestines, kidney and brain.

In yet another embodiment, the present disclosure provides compounds for use in the treatment and/or prevention of CCR4 activity that promotes migration of leukaemia cells to sites resulting in development of cutaneous or thymic lymphomas through recruitment of immunosuppressive regulatory T-cells (Treg) cells that can inhibit tumor immunity and clearance.

According to yet another embodiment, the disclosure provides the use of the compounds of the present disclosure in the manufacture of a medicament for the treatment of diseases and/or disorder responsive to the modulation of CCR4 activity.

According to yet another embodiment, the disclosure provides a compound or a pharmaceutical acceptable salt or a stereoisomer thereof, for use in the treatment of CCR4 mediated disease or disorder. In yet another embodiment, the disclosure provides the use of the compounds of the present disclosure in the manufacture of a medicament for the treatment of cancer or an inflammatory disease or disorder.

In yet another embodiment, the disclosure provides the use of the compounds of the present disclosure in the manufacture of a medicament for the treatment of cancer.

In yet another embodiment, the disclosure provides the use of the compounds of the present disclosure in the manufacture of a medicament for the treatment of cancer wherein the cancer is colon cancer, pancreatic cancer, intestinal cancer, breast cancer, lung cancer, gastric cancer, liver cancer or colorectal cancer.

In yet another embodiment, the disclosure provides the use of the compounds of the present disclosure in the manufacture of a medicament for the treatment of inflammatory disease or disorder.

In yet another embodiment, the disclosure provides the use of the compounds of the present disclosure in the manufacture of a medicament for the treatment of inflammatory disease or disorder wherein the inflammatory disease or disorder is dermatitis. In yet another embodiment, the disclosure provides the use of the compounds of the present disclosure in the manufacture of a medicament for the treatment of inflammatory disease or disorder wherein the inflammatory disease or disorder is atopic dermatitis or contact dermatitis.

In yet another embodiment, the present application provides compounds for use as a medicament for treating a subject suffering from diseases and/or disorder responsive to the modulation of CCR4 activity.

In yet another embodiment, the present application provides compounds for use in the manufacture of a medicament for treating a subject suffering from diseases and/or disorder responsive to the modulation of CCR4 activity.

In yet another embodiment, the disclosure provides compounds of the present disclosure for use in the manufacture of a medicament for the treatment of cancer.

In yet another embodiment, the disclosure provides compounds of the present disclosure for use in the manufacture of a medicament for the treatment of inflammation.

In yet another embodiment, the disclosure provides compounds of the present disclosure for use in the manufacture of a medicament for the treatment of cancer wherein the cancer is colon cancer, pancreatic cancer, intestinal cancer, breast cancer, lung cancer, gastric cancer, liver cancer or colorectal cancer.

In yet another embodiment, the disclosure provides compounds of the present disclosure for use in the manufacture of a medicament for the treatment of inflammatory disease or disorder.

In yet another embodiment, the disclosure provides compounds of the present disclosure for use in the manufacture of a medicament for the treatment of inflammatory disease or disorder wherein the inflammatory disease or disorder is dermatitis.

In yet another embodiment, the disclosure provides compounds of the present disclosure for use in the manufacture of a medicament for the treatment of inflammatory disease or disorder wherein the inflammatory disease or disorder is atopic dermatitis or contact dermatitis.

In yet another embodiment, the present disclosure comprises administering to the subject in need thereof a therapeutically effective amount of a compound of the present disclosure along with one or more additional chemotherapeutic agents independently selected from anti-proliferative agents, anti-cancer agents, immunosuppressant agents and pain-relieving agents.

In yet another embodiment, the present disclosure comprises administering to the subject in need thereof a therapeutically effective amount of a compound of the present disclosure along with one or more additional anti-inflammatory agents.

The method(s) of treatment of the present disclosure comprises administering a safe and effective amount of a compound of formula (I) or a pharmaceutically acceptable salt thereof to a patient (particularly a human) in need thereof.

In certain embodiments, the present disclosure provides a method of treating diseases and/or disorder mediated by CCR4 in a subject comprising administering to the subject in need thereof a therapeutically effective amount of a compound of formula (I).

In certain embodiments, the present disclosure provides a method of modulating CCR4 in a subject, comprising contacting CCR4 with a compound of formula (I), or a pharmaceutically acceptable salt thereof.

In certain embodiments, the present disclosure provides a method of treating or preventing a disease or disorder mediated by CCR4, comprising administering to the subject in need thereof a therapeutically effective amount of a compound of formula (I), or a pharmaceutically acceptable salt thereof.

In certain embodiments, the present disclosure provides a method of treating or preventing a disease or disorder mediated by CCR4, the disease or disorder is cancer or an inflammatory disease or disorder.

According to certain foregoing embodiments, the present disclosure provides a method wherein the disease or disorder is inflammation.

According to certain foregoing embodiments, the present disclosure provides a method wherein the disease or disorder is dermatitis.

According to certain foregoing embodiments, the present disclosure provides a method wherein the disease or disorder is atopic dermatitis or contact dermatitis.

According to certain foregoing embodiments, the present disclosure provides a method wherein the disease or disorder is cancer.

According to certain foregoing embodiments, the present disclosure provides a method wherein the cancer is colon cancer, pancreatic cancer, intestinal cancer, breast cancer, lung cancer, gastric cancer, liver cancer or colorectal cancer.

According to certain foregoing embodiments, the present disclosure provides a method comprising administering to the subject in need thereof a therapeutically effective amount of a compound of the present disclosure along with one or more chemotherapeutic agents or anti-inflammatory agents.

Compounds of the disclosure are indicated both in the therapeutic and/or prophylactic treatment of the above-mentioned conditions. For the above-mentioned therapeutic uses the dosage administered will, of course, vary with the compound employed, the mode of administration, the treatment desired and the disorder or disease indicated.

The compounds of the present disclosure may be used as single drug or as a pharmaceutical composition in which the compound is mixed with various pharmacologically acceptable materials.

According to one embodiment, the present disclosure provides compounds for use in combination with other compounds or biologic entities for treatment of inflammatory diseases or cancer. Suitable combinations and doses of compounds for combination therapy used in treating the diseases or disorders described herein can be determined by those skilled in the relevant art. Combination therapies for compounds of the present disclosure can be used for treatment of inflammatory diseases or cancer.

According to one embodiment, the compounds of the present disclosure can also contain unnatural proportions of atomic isotopes at one or more of the atoms that constitute such compounds. For example, the present disclosure also embraces isotopically-labeled variants of the present disclosure which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the disclosure and their uses. Exemplary isotopes that can be incorporated in to compounds of the disclosure include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²p, ³³p, ³⁵S, ¹⁸F, ³⁶Cl, ¹²³I and ¹²⁵I. Isotopically labeled compounds of the present disclosure can generally be prepared by following procedures analogous to those disclosed in the schemes and/or in the examples herein below, by substituting an isotopically labeled reagent for a non-isotopically labeled reagent.

### EXPERIMENTAL

The present application provides methods for the preparation of compound of formula (I) according to the description provided herein using appropriate methods and/or materials. It is to be understood by those skilled in the art that known variations of the conditions and processes of the following procedures can be used to prepare these intermediates and compounds. Moreover, by utilizing the procedures described in detail, one of ordinary skill in the art can prepare additional compounds of the present disclosure.

Following general guidelines apply to all experimental procedures described here. Until otherwise stated, experiments are performed under positive pressure of nitrogen, temperature described are the external temperature (i.e. oil bath temperature). Reagents and solvents received from vendors are used as such without any further drying or purification. Molarities mentioned here for reagents in solutions are approximate as it was not verified by a prior titration with a standard. All reactions are stirred under magnetic stir bar. Cooling to minus temperature was done by acetone/dry ice or wet ice/salts. Magnesium sulfate and sodium sulfate were used as solvent drying agent after reaction work up and are interchangeable.

Removing of solvents under reduced pressure or under vacuum means distilling of solvents in rotary evaporator.

Compounds of this disclosure may be made by synthetic chemical processes, examples of which are shown herein. It is meant to be understood that the order of the steps in the processes may be varied, that reagents, solvents and reaction conditions may be substituted for those specifically mentioned and that vulnerable moieties may be protected and deprotected, as necessary.

The specifics of the process for preparing compounds of the present disclosure are detailed in the experimental section.

The present disclosure shall be illustrated by means of some examples, which are not construed to be viewed as limiting the scope of the disclosure.

Unless otherwise stated, work-up includes distribution of the reaction mixture between the organic and aqueous phases, separation of layers and drying the organic layer over anhydrous sodium sulphate, filtration and evaporation of the solvent. Purification, unless otherwise mentioned, includes purification by silica gel chromatographic techniques, generally using ethyl acetate/petroleum ether mixture of a suitable polarity as the mobile phase.

Analysis for the compounds of the present disclosure unless mentioned, was conducted in general methods well known to a person skilled in the art. Having described the disclosure with reference to certain preferred embodiments, other embodiments will become apparent to one skilled in the art from consideration of the specification. The disclosure is further defined by reference to the following examples, describing in detail the analysis of the compounds of the disclosure.

It will be apparent to those skilled in the art that many modifications, both to materials and methods, may be practiced without departing from the scope of the disclosure. Some of the intermediates were taken to next step based on TLC results, without further characterization, unless otherwise specified.

### Abbreviations

DIPA—diisopropylamine, NBS—N-Bromosuccinimide, DMF—N,N-Dimethylformamide; ACN—Acetonitrile; EtOAc—Ethyl acetate; THF—Tetrahydrofuran; ACOH—Acetic acid, TFAA—Trifluoroacetic anhydride, Et₂O—Diethyl ether, MeMgBr—Methyl magnesium bromide, mCPBA—meta-Chloroperoxybenzoic acid, 1,2-DME—1,2-Dimethoxyethane, MeI—methyl iodide, DCM—Dichloromethane; MeNH2—methylamine, MeOH—Methanol; EtOH—Ethanol; DMSO—dimethyl sulfoxide; MeI—Methyl iodide, NaOtBu—Sodium tert-butoxide, KOtBu—Potassium tert-butoxide t-BuOH—tert-Butyl alcohol, CsF—Cesium fluoride, BINAP—2,2′-bis(diphenylphosphino)-1,1′-binaphthyl, DEA—Diethanolamine, DIPEA-N,N-Diisopropylethylamine, TFA—Trifluoroacetic acid; TLC—Thin layer chromatography; LC-MS—Liquid chromatography—mass spectrometry; HPLC—High performance liquid chromatography; DMSO-d₆—Deuterated dimethyl sulfoxide; NMR—Nuclear magnetic resonance, s—Singlet, d—Doublet, t—Triplet, q—Quartet, app. quint—Apparent quintet, dd—doublet of doublets, td—Triplet of doublets, SM—starting material, Int—Intermediate, Comp.—Compound, RB—Round bottom, RT—Room temperature, MHz—Megahertz, RM—Reaction mixture, DMP—Dess martin periodinane, Et₃N—Triethylamine.

### SYNTHESIS OF INTERMEDIATES

### Intermediate I-1: Synthesis of 7-bromo-6-chlorobenzo[d]oxazole-2-thiol

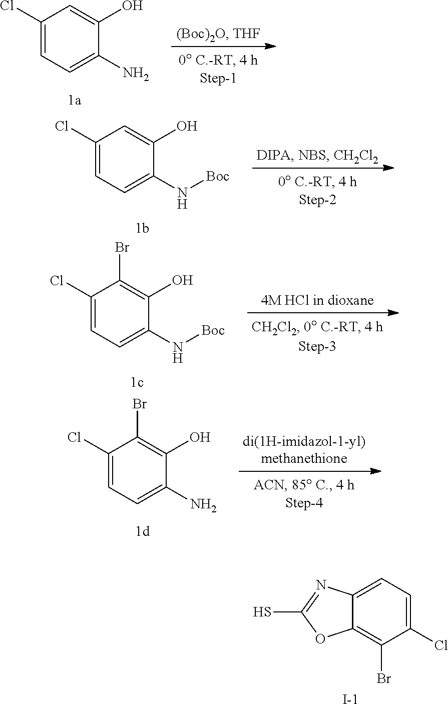 [SMILES: Nc1ccc(Cl)cc1O ; CC(C)(C)OC(=O)Nc1ccc(Cl)cc1O ; CC(C)(C)OC(=O)Nc1ccc(Cl)c(Br)c1O ; Nc1ccc(Cl)c(Br)c1O ; Sc1nc2ccc(Cl)c(Br)c2o1 ; [I-]]

### Step-1: Synthesis of tert-butyl (4-chloro-2-hydroxyphenyl)carbamate (1b)

To the stirred solution of 2-amino-5-chlorophenol (5 g, 34.8 mmol) in THE (50 mL) was added Boc anhydride (11.4 g, 52.2 mmol) at 0° C. The resulting reaction mixture was warmed to RT and stirred at the same temperature for 4 h. After the completion of reaction, diluted with ice cold water, extracted with ethyl acetate and layers were separated. The organic layer was dried over anhydrous Na₂SO₄, filtered, and concentrated to get crude compound. The crude compound was purified by combi-flash chromatography using 10% ethyl acetate in hexane as an eluent to afford the pure title compound 1b (5 g, 58.9% yield). LC-MS: 242.0 [M−H].

### Step-2: Synthesis of tert-butyl (3-bromo-4-chloro-2-hydroxyphenyl)carbamate (1c)

To the stirred solution of tert-butyl (4-chloro-2-hydroxyphenyl)carbamate (0.5 g, 2.052 mmol) in CH₂Cl₂ (10 mL) was added diisopropylamine (0.441 g, 4.1 mmol) and NBS (0.29 g, 1.64 mmol) at 0° C. The reaction mixture was warmed to RT and stirred for 4 hrs. After the completion of reaction, quenched with saturated aq. sodium thiosulphate solution, extracted with CH₂Cl₂ and the layers were separated. The organic layer was dried over anhydrous Na₂SO₄, filtered, and concentrated to get the crude. The crude compound was purified by combi flash chromatograph using 5% ethyl acetate in hexane as an eluent to afford the pure title compound 1c (0.2 g, 30.2%). LC-MS: 321.9 [M−H]⁻.

### Step-3: Synthesis of 6-amino-2-bromo-3-chlorophenol (1d)

To the stirred solution of tert-butyl (3-bromo-4-chloro-2-hydroxyphenyl)carbamate (0.12 g, 0.37 mmol) in CH₂Cl₂ (1 mL) was added 4M HCl in dioxane (1 mL) at 0° C. Then the resulting reaction mixture was stirred at RT for 4 h. After the completion of reaction, quenched with saturated aq. NaHCO₃ solution, extracted with ethyl acetate and the layers were separated. The organic layer was dried over anhydrous Na₂SO₄, filtered, and concentrated to afford the title compound 1d (0.1 g). LC-MS: 223.9 [M+H]⁺.

### Step-4: Synthesis of 7-bromo-6-chlorobenzo[d]oxazole-2-thiol (I-1)

To the stirred solution of 6-amino-2-bromo-3-chlorophenol (0.5 g, 2.24 mmol) in acetonitrile (10 mL) was added di(1H-imidazol-1-yl)methanethione (0.8 g, 4.49 mmol) at 0° C. The resulting reaction mixture was warmed to RT and stirred at 85° C. for 4 h. After the completion of reaction, cooled to RT, quenched with 1N HCl, extracted with ethyl acetate and the layers were separated. The organic layer was dried over anhydrous Na₂SO₄, filtered, and concentrated to afford the title compound I-1 (0.55 g). LC-MS: 263.8 [M−H]⁻.

### Intermediate I-2: Synthesis of 7-bromobenzo[d]oxazole-2-thiol

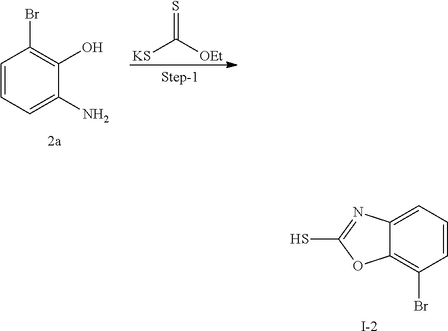

### Step-1: 7-bromobenzo[d]oxazole-2-thiol (I-2)

To the stirred solution of 2-amino-6-bromophenol (3 g, 15.9 mmol) in methanol (50 mL) was added potassium ethyl xanthate (2.83 g, 17.8 mmol) at RT and resulting reaction mixture was stirred at 65° C. for 7 h. After the completion of reaction, cooled to RT, solvent was removed under vacuum pressure, the residue was dissolved in water and acidified with 1N HCl. The precipitated solid was filtered, washed with water. The residue thus obtained was dissolved using ethyl acetate, washed with water and the two layers were separated. The organic layer was dried over anhydrous Na₂SO₄, filtered, and concentrated under vacuo to get dark brown solid I-2 (2.36 g). This was used as such without further purification. LC-MS: 229.8 [M−H]⁻.

The intermediates listed below in Table-A were prepared using similar procedure described in the synthesis of I-1 & I-2 with appropriate variations in cyclization methods, reactants, quantities of reagents and solvents, and reaction conditions. The characterization data of the compounds are summarized herein the below table.

TABLE A

| Int. No. | Starting material | Intermediate | Spectral data |
|---|---|---|---|
| A-1 | 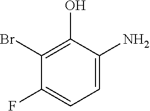 | 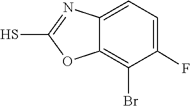 [SMILES: Fc1ccc2nc(S)oc2c1Br] | LC-MS: 247.1 [M − H]⁻ |
| A-2 | 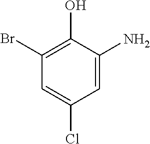 | 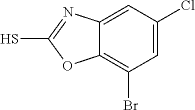 [SMILES: Sc1nc2cc(Cl)cc(Br)c2o1] | LC-MS: 263.5 [M − H]⁻ |
| A-3 | 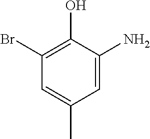 | 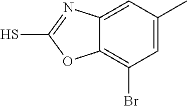 | LC-MS: 243.1 [M − H]⁻ |
| A-4 | 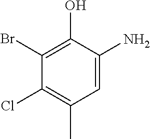 | 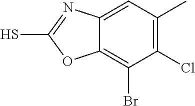 | LC-MS: 277.5 [M − H]⁻ |
| A-5 | 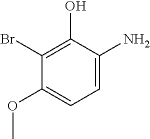 | 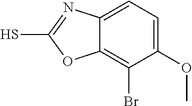 | LC-MS: 259.1 [M − H]⁻ |
| A-6 | 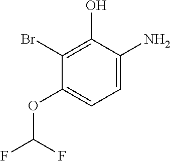 [SMILES: Nc1ccc(OC(F)F)c(Br)c1O] | 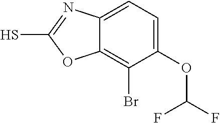 [SMILES: FC(F)Oc1ccc2nc(S)oc2c1Br] | LC-MS: 295.1 [M − H]⁻ |
| A-7 | 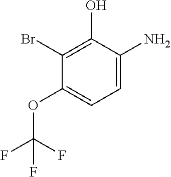 [SMILES: Nc1ccc(OC(F)(F)F)c(Br)c1O] | 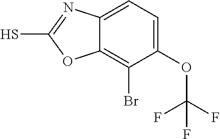 [SMILES: FC(F)(F)Oc1ccc2nc(S)oc2c1Br] | LC-MS: 313.1 [M − H]⁻ |
| A-8 | 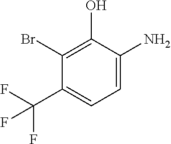 [SMILES: Nc1ccc(C(F)(F)F)c(Br)c1O] | 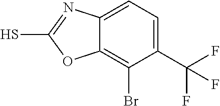 [SMILES: FC(F)(F)c1ccc2nc(S)oc2c1Br] | LC-MS: 297.1 [M − H]⁻ |
| A-9 | 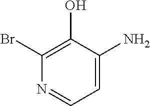 | 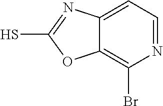 [SMILES: Sc1nc2ccnc(Br)c2o1] | LC-MS: 230.1 [M − H]⁻ |
| A-10 | 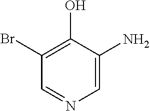 | 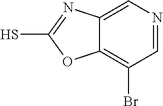 [SMILES: Sc1nc2cncc(Br)c2o1] | LC-MS: 230.1 [M − H]⁻ |
| A-11 | 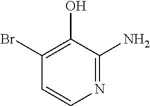 | 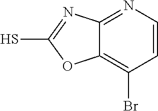 [SMILES: Sc1nc2nccc(Br)c2o1] | LC-MS: 230.1 [M − H]⁻ |
| A-12 | 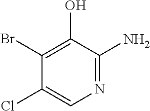 | 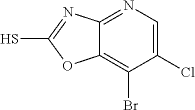 [SMILES: Sc1nc2ncc(Cl)c(Br)c2o1] | LC-MS: 264.5 [M − H]⁻ |
| A-13 | 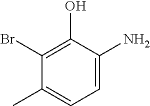 | 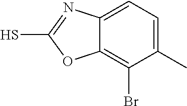 | LC-MS: 243.1 [M − H]⁻ |
| A-14 | 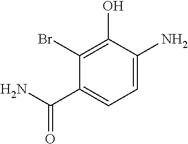 [SMILES: NC(=O)c1ccc(N)c(O)c1Br] | 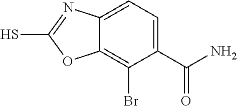 [SMILES: NC(=O)c1ccc2nc(S)oc2c1Br] | LC-MS: 272.1 [M − H]⁻ |
| A-15 | 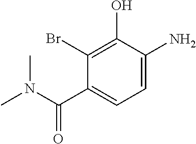 | 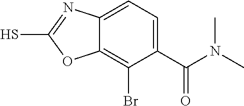 [SMILES: CN(C)C(=O)c1ccc2nc(S)oc2c1Br] | LC-MS: 300.2 [M − H]⁻ |
| A-16 | 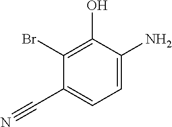 [SMILES: N#Cc1ccc(N)c(O)c1Br] | 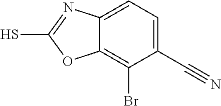 [SMILES: N#Cc1ccc2nc(S)oc2c1Br] | LC-MS: 254.1 [M − H]⁻ |
| A-17 | 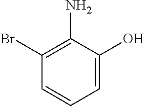 | 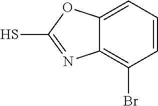 [SMILES: Sc1nc2c(Br)cccc2o1] | LC-MS: 229.8 [M − H]⁻ |

### Intermediate I-3: Synthesis of 4-bromo-2-chloro-1-methyl-1H-benzo[d]imidazole

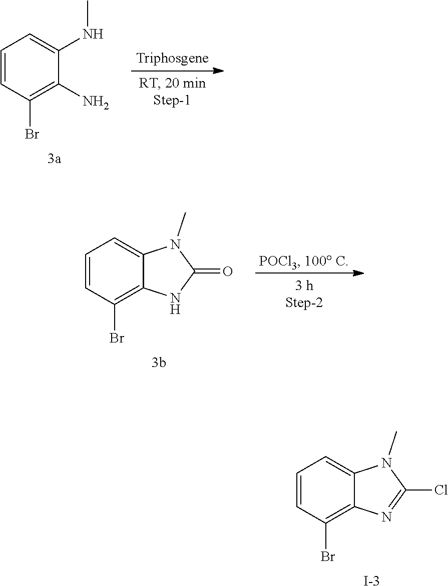

### Step-1: Synthesis of 4-bromo-1-methyl-1,3-dihydro-2H-benzo[d]imidazol-2-one (3b)

To the stirred solution of 3-bromo-N¹-methylbenzene-1,2-diamine (0.75 g, 3.73 mmol) in THE (15 mL) was added triphosgene (0.332 g, 1.11 mmol) at RT. The resulting reaction mixture was stirred for 20 minutes at the same temperature. After the completion of reaction, the reaction mixture was quenched with water, extracted with ethyl acetate and the layers were separated. The organic layer was dried over anhydrous Na₂SO₄, filtered, and concentrated to get the title compound 3b (0.79 g). LC-MS: 228.8 [M+H]⁺.

### Step-2: Synthesis of 4-bromo-2-chloro-1-methyl-1H-benzo[d]imidazole (1-3)

In an RB flask, mixture of 4-bromo-1-methyl-1,3-dihydro-2H-benzo[d]imidazol-2-one (1.5 g, 6.6 mmol) and POCl₃ (20.2 g) was heated to 100° C. for 3 h. After the completion of reaction, cooled to RT, quenched with ice cold water, extracted with ethyl acetate, and the layers were separated. The organic layer was dried over anhydrous Na₂SO₄, filtered, and concentrated to get the title compound I-3 (0.8 g, 49.3%). ¹H-NMR (400 MHz, CDCl₃): δ 7.5 (d, 1H), 7.48 (d, 1H), 7.26-7.18 (m, 1H), 3.82 (s, 3H).

### Intermediate 1-4: Synthesis of 7-bromo-2-chloro-1-methyl-1H-benzo[d]imidazole

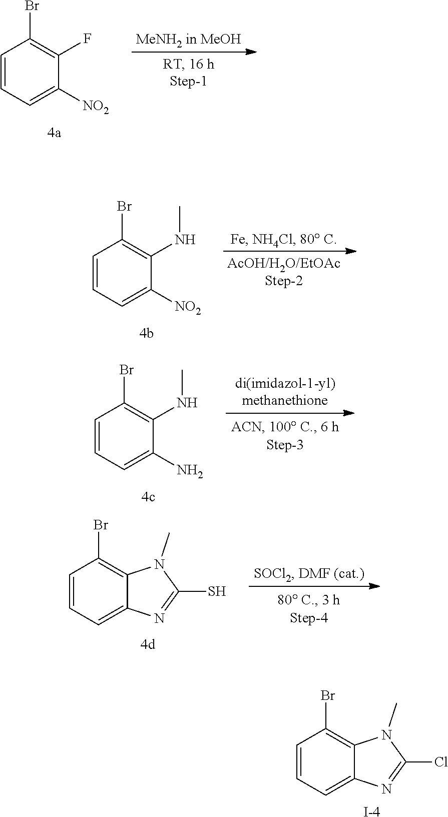

### Step-1: Synthesis of 2-bromo-N-methyl-6-nitroaniline (4b)

Compound 4a (9 g, 40.9 mmol) in methylamine in MeOH (90 mL) was stirred at room temperature for 16 hrs. Progress of the reaction was monitored by TLC. After completion of reaction concentrated under reduced pressure to get crude solid 4b (10 g crude yield) which was used without any further purification. LC-MS: 232.1 [M+H]⁺.

### Step-2: Synthesis of 6-bromo-N¹-methylbenzene-1,2-diamine (4c)

To a stirred solution of 2-bromo-N-methyl-6-nitroaniline (4b) (3 g, 12.9 mmol) in AcOH: EtOAc: water (3:1:0.1, 42 mL) was added iron powder (2.9 g, 51.9 mmol) and heated at 80° C. for 1 h. Progress of the reaction was monitored by TLC. After completion of the reaction quenched by ice water and extracted with CH₂Cl₂. The organic layer was washed by brine solution dried over anhydrous Na₂SO₄ and concentrated under reduced pressure to get tittle compound 4c (1.6 g, 62% yield). LC-MS: 202.2 [M+H]⁺.

### Step-3: Synthesis of 7-bromo-1-methyl-1H-benzo[d]imidazole-2-thiol (4d)

To a stirred solution of 6-bromo-N¹-methylbenzene-1,2-diamine (4c) (1.6 g, 7.9 mmol) in ACN (60 mL), 1 1′-carbonyldiimidazole (2.67, 14.9 mmol) was added and stirred at 100° C. for 6 hrs. Progress of the reaction was monitored by TLC. After completion of starting material, the reaction mixture was quenched with 1(N) HCl and extracted with ethyl acetate and concentrated to get tittle compound 4d (1.3 g crude yield). LC-MS: 244.1 [M+H]⁺.

### Step-4: Synthesis of 7-bromo-2-chloro-1-methyl-1H-benzo[d]imidazole (I-4)

7-bromo-1-methyl-1H-benzo[d]imidazole-2-thiol (4d) (0.5 g, 2.05 mmol) was dissolved in thionyl chloride (2.1 mL, 28.7 mmol) under argon at RT followed by 2 drops of DMF was added as catalyst and the reaction was heated at 80° C. for 30 mins. After cooling down to RT toluene was added and the mixture was evaporated to remove the excess of SOCl₂. The residue was poured into cold water, and it was extracted with toluene. The organic phase was dried over Na₂SO₄, filtered, and evaporated to get the crude which was Purified by flash chromatography using 15% ethyl acetate in hexane to get title compound I-4 (0.4 g, 85% yield). LC-MS: 246.5 [M+H]⁺.

### Intermediate 1-5: Synthesis of 7-bromobenzo[d]thiazole-2-thiol

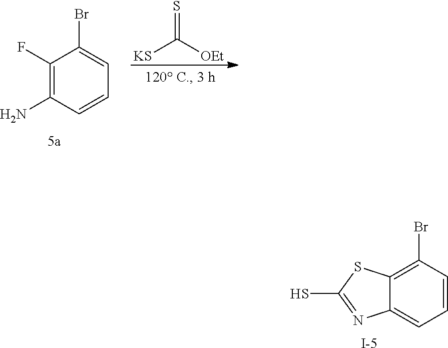

To the stirred solution of 5a (2 g, 10.52 mmol) in DMF (20 mL) was added potassium ethyl xanthate (3.73 g, 23.1 mmol) at RT. The resulting reaction mixture was stirred at 120° C. for 3 h. After the completion of reaction, the reaction mixture was cooled to RT and acidified with 1N HCl. The precipitated solid was filtered, washed with water, and dried to get the title compound (1.9 g crude yield). This was used as such in the next step. LC-MS: 248.0 [M+H+1]⁺.

The intermediates listed below in Table-B were prepared using similar procedure described in the synthesis of I-5 with appropriate variations in cyclization methods, reactants, quantities of reagents and solvents, and reaction conditions. The characterization data of the compounds are summarized herein the below table.

TABLE B

| Int. No. | Starting material | Intermediate | Spectral data |
|---|---|---|---|
| A-18 | 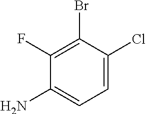 | 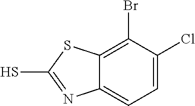 [SMILES: Sc1nc2ccc(Cl)c(Br)c2s1] | LC-MS: 282.6 [M + H + 1]⁺ |
| A-19 | 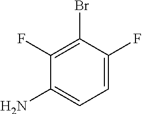 | 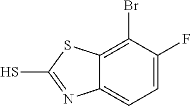 [SMILES: Fc1ccc2nc(S)sc2c1Br] | LC-MS: 266.1 [M + H + 1]⁺ |
| A-20 | 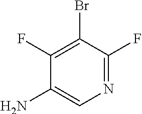 | 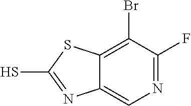 [SMILES: Fc1ncc2nc(S)sc2c1Br] | LC-MS: 267.1 [M + H + 1]⁺ |
| A-21 | 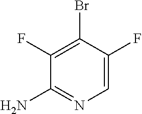 | 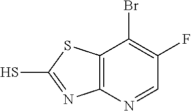 [SMILES: Fc1cnc2nc(S)sc2c1Br] | LC-MS: 267.1 [M + H + 1]⁺ |
| A-22 | 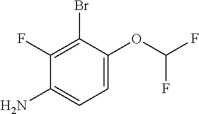 [SMILES: Nc1ccc(OC(F)F)c(Br)c1F] | 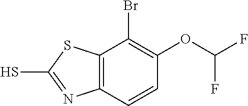 [SMILES: FC(F)Oc1ccc2nc(S)sc2c1Br] | LC-MS: 314.1 [M + H + 1]⁺ |

### Intermediate T-6

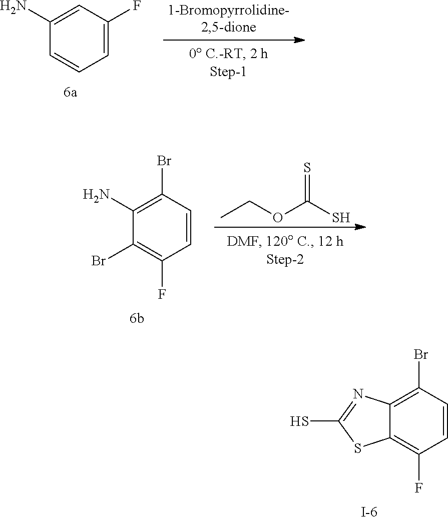

### Step-1: Synthesis of 2,6-dibromo-3-fluoroaniline (6b)

To a stirred solution of 3-fluoroaniline (1 g, 8.99 mmol) in acetonitrile (30 mL), 1-bromopyrrolidine-2,5-dione (3.2 g, 17.9 mmol) was added at 0° C. and stirred at RT for 2 hrs. After the completion of the reaction concentrated under reduced pressure to get the crude which was purified by flash chromatography using 1% ethyl acetate in hexane as an eluent to afford the pure title compound 6b (2 g, 82.5% yield). LC-MS: 269.9 [M+H]⁺.

### Step-2: Synthesis of 4-bromo-7-fluorobenzo[d]thiazole-2-thiol (1-6)

To a stirred solution of 2,6-dibromo-3-fluoroaniline (6b) (2 g, 7.44 mmol) in DMF (20 mL), ethyl xanthic acid (2 g, 16.36 mmol) was added and stirred at 120° C. for 12 hrs. After the completion of reaction, the reaction mixture quenched with 1N HCl solution to get the solid which was filtered to pure title compound I-6 used for next step without further purification. LC-MS: 266.1 [M+1+H]⁺.

### Intermediate I-7: Synthesis of (1R,3r)-3-((R)-3-(azetidin-3-yl)piperidin-1-yl)-1-methylcyclobutane-1-carboxylic acid

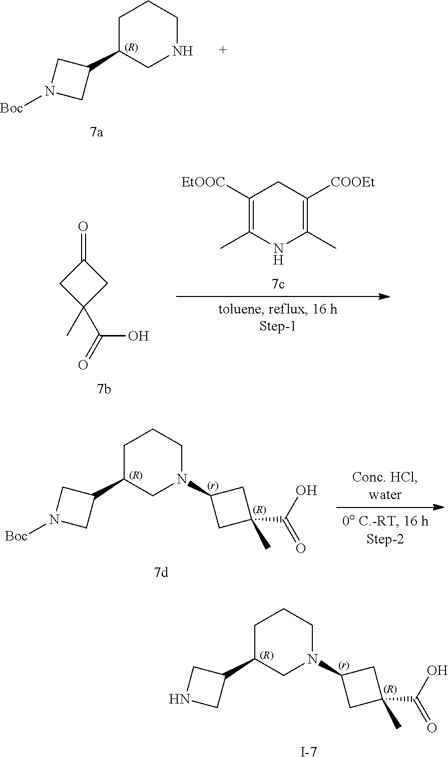

### Step-1: Synthesis of (1R,3r)-3-((R)-3-(1-(tert-butoxycarbonyl)azetidin-3-yl)piperidin-1-yl)-1-methylcyclobutane-1-carboxylic acid (7d)

To a stirred solution of tert-butyl (R)-3-(piperidin-3-yl)azetidine-1-carboxylate (7a) (15 g, 62.4 mmol), 1-methyl-3-oxocyclobutane-1-carboxylic acid (7b) (6.39 g, 49.9 mmol) in toluene (300 mL) was added diethyl 2,6-dimethyl-1,4-dihydropyridine-3,5-dicarboxylate (7c) (18.9 g, 74.8 mmol) and the mixture was refluxed in a round bottom fitted with Dean stark condenser for 16 hrs. The reaction mixture was cooled to room temperature, quenched with water, washed with ethyl acetate and the aqueous layer was concentrated to get the title compound 7d (21 g crude yield). LC-MS: 353.15 [M+H]⁺.

### Step-2: (1R,3r)-3-((R)-3-(azetidin-3-yl)piperidin-1-yl)-1-methylcyclobutane-1-carboxylic acid (I-7)

To a solution of (1R,3r)-3-((R)-3-(1-(tert-butoxycarbonyl)azetidin-3-yl)piperidin-1-yl)-1-methylcyclobutane-1-carboxylic acid (7d) (20 g, 56.7 mmol) in water (160 mL) was added con. HCl (40 mL) at 0° C. The reaction mixture was gradually warmed to RT and stirred for 16 h. The reaction mixture was then washed with diethyl ether followed by ethyl acetate and the aqueous portion was neutralized with 6N aq. NaOH solution. The aqueous portion was concentrated, and the residue obtained was added 20% methanol in CH₂Cl₂ and filtered. The filtrate was concentrated, and the residue was washed with diethyl ether to afford title compound I-7 in quantitative yield. LC-MS: 253.2 [M+H]⁺; ¹H-NMR (400 MHz, DMSO-d⁶) 3.70-3.59 (m, 2H), 3.55-3.45 (m, 2H), 2.67-2.52 (m, 2H), 2.48-2.30 (m, 3H), 1.68-1.42 (m, 6H), 1.4-1.2 (m, 3H), 1.77 (s, 3H), 0.80-0.65 (m, 1H).

### Intermediate A-23: 2-(1-(3-(azetidin-3-yl)piperidin-1-yl)cyclobutyl)acetic acid hydrochloride

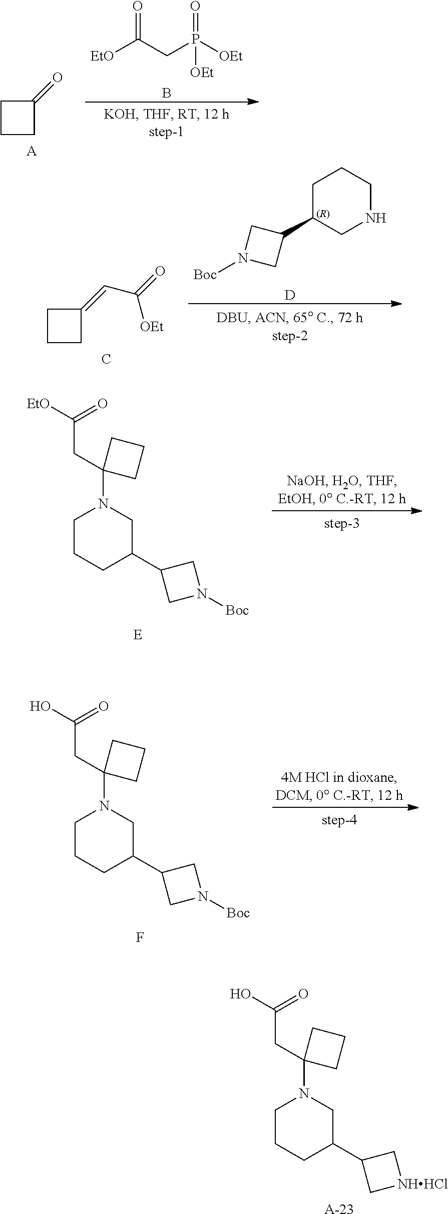

### Step-1: Synthesis of ethyl 2-cyclobutylideneacetate

To a stirred solution of reagent A in THF was added ethyl 2-diethoxyphosphorylacetate (B) and potassium hydroxide at 0° C. under argon atmosphere. Reaction mixture slowly warmed to RT and stirred for 12 h. Then the reaction was monitored by TLC and after completion, quenched with water and extracted with diethyl ether. The combined organic layer was concentrated to get crude intermediate C of compound A-23 which was used for the next step.

LC-MS: 141.2 [M+H]⁺

### Step-2: Synthesis of tert-butyl 3-(1-(1-(2-ethoxy-2-oxoethyl)cyclobutyl)piperidin-3-yl)azetidine-1-carboxylate

To a stirred solution of intermediate C and D in ACN was added 2,3,4,6,7,8,9,10-Octahydropyrimido[1,2-a]azepine and stirred at 65° C. under argon atmosphere for 72 h. After the completion of reaction monitored by TLC, evaporated to dryness to get crude which was purified by combi flash column chromatography, eluted with 0-20% MeOH:DCM to get pure intermediate E of compound A-23. LC-MS: 381.5 [M+H]⁺

### Step-3: Synthesis of 2-(1-(3-(1-(tert-butoxycarbonyl)azetidin-3-yl)piperidin-1-yl)cyclobutyl)acetic acid

To a stirred solution of intermediate E in THF:EtOH:H₂O (2:2:1) was added sodium hydroxide (3 eq.) at 0° C. and stirred for 12 h at RT. After the completion of reaction, reaction mixture was concentrated under reduced pressure and washed with 1N HCl extracted with 10% MeOH:DCM. The combined organic layer was concentrated to get crude intermediate F of compound A-23 which was used for the next step. LC-MS: 353.5 [M+H]⁺

### Step-4: Synthesis of 2-(1-(3-(azetidin-3-yl)piperidin-1-yl)cyclobutyl)acetic acid hydrochloride

To a stirred solution of intermediate F in DCM at 0° C. was added 4M HCl in 1 4-dioxane under argon atmosphere. The resulting reaction mixture was stirred at RT for 12 h. After the completion of reaction, concentrated under reduced pressure to get crude salt A-23 which was used for next step. LC-MS: 252.5 [M]⁺

### Intermediate A-24: Synthesis of (1r,3r)-3-(2-(azetidin-3-yl)morpholino)-1-methylcyclobutane-1-carboxylic acid

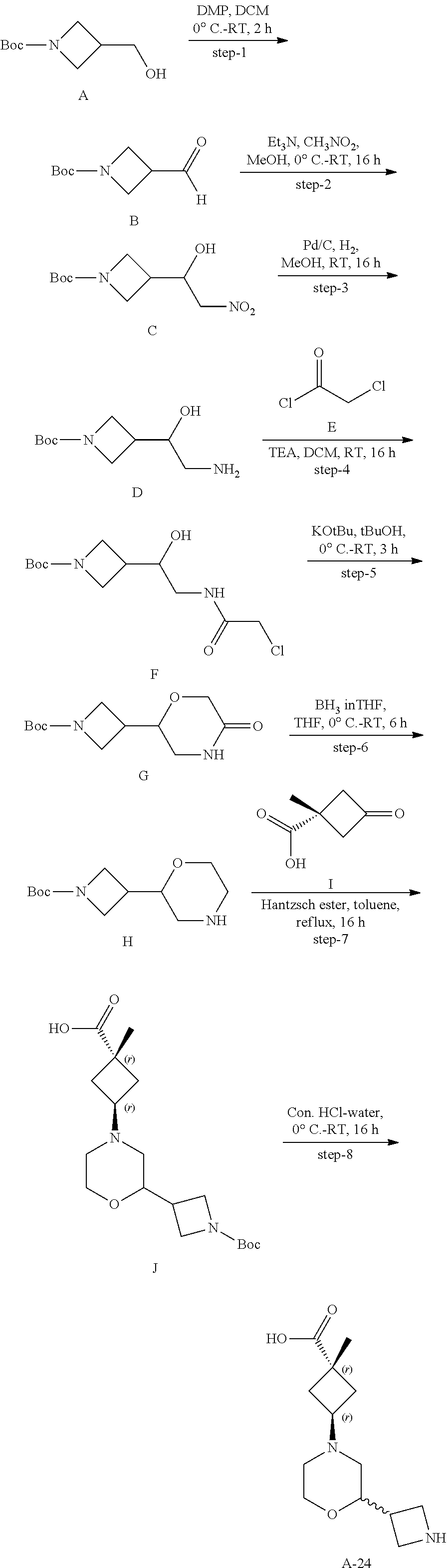

### Step-1: Synthesis of tert-butyl 3-formylazetidine-1-carboxylate

To the stirred solution of tert-butyl 3-(hydroxymethyl) azetidine-1-carboxylate (5 g, 26.70 mmol) in DCM (50 mL) was added Dess martin periodinane (13.59 g, 32.04 mmol) at 0° C. The resulting reaction mixture was stirred at RT for 2 h. The reaction was monitored by TLC and showed that absence of starting material. The reaction mixture was cooled to 0° C. and reaction mixture was poured in saturated NaHCO₃ solution and extracted with DCM, organic layer was washed with brine and dried over on anhydrous Na₂SO₄ and concentrated under reduced pressure to afford the crude material. The crude material was purified by flash chromatography and eluted with 0-60% EtOAc in hexane to afford Intermediate B of compound A-24 (4.5 g, 91%). ¹H NMR (400 MHz, CDCl₃) δ 9.86 (s, 1H), 4.164-4.081 (m, 4H), 1.460 (S, 9H)

### Step-2: Synthesis of tert-butyl 3-(1-hydroxy-2-nitroethyl) azetidine-1-carboxylate

To the stirred solution of tert-butyl 3-formylazetidine-1-carboxylate (4.5 g, 24.25 mmol) in MeOH (50 mL) was added Triethylamine (7.3 g, 72.80 mmol) and Nitromethane (4.45 g, 72.88 mmol) at 0° C. The resulting reaction mixture was stirred RT for 16 h. The reaction was monitored by TLC and showed that absence of starting material. The reaction mixture was concentrated under reduced pressure to afford the crude material. Crude material was purified by flash chromatography and eluted with 0-60% EtOAc in hexane to afford Intermediate C of compound A-24 (4.40 g, 91%). LC-MS: 245.0 [M+H]⁺

### Step-3: Synthesis of tert-butyl 3-(2-amino-1-hydroxyethyl) azetidine-1-carboxylate

To the stirred solution of tert-butyl 3-(1-hydroxy-2-nitroethyl) azetidine-1-carboxylate (4 g, 16.24 mmol) in MeOH (50 mL) was added Pd/C (10%) (1.3 g) at RT. The resulting reaction mixture was stirred at RT under H₂ balloon pressure (30 psi) for 16 h. The reaction was monitored by TLC and showed that absence of starting material and reaction mixture was filtered through celite pad and washed further with excess of MeOH. Filtrate was concentrated under reduced pressure to afford the crude intermediate D of compound A-24 (4.0 g). LC-MS: 217.2 [M+H]⁺

### Step-4: Synthesis of tert-butyl 3-(2-(2-chloroacetamido)-1-hydroxyethyl) azetidine-1-carboxylate

To the stirred solution of tert-butyl 3-(2-amino-1-hydroxyethyl) azetidine-1-carboxylate (3.7 g, 17.11 mmol) in DCM (40 mL) was added Triethylamine (5.19 g, 51.32 mmol) and Chloro acetyl chloride (2.13 g, 18.81 mmol) at 0° C. The resulting reaction mixture was stirred at RT for 16 h. The reaction was monitored by TLC and showed that absence of starting material. The reaction mixture was cooled to 0° C. and reaction mixture was poured in saturated NaHCO₃ solution and extracted with DCM, organic layer was washed with brine and dried over on anhydrous Na₂SO₄ and concentrated under reduced pressure to afford the crude compound. The crude compound was purified by flash chromatography and eluted with 0-80% EtOAc in hexane to afford the intermediate F of compound A-24 (2.8 g 56%). LC-MS: 193.1[M-Boc]⁺

### Step-5: Synthesis of tert-butyl 3-(5-oxomorpholin-2-yl) azetidine-1-carboxylate

To the stirred solution of tert-butyl 3-(2-(2-chloroacetamido)-1-hydroxyethyl) azetidine-1-carboxylate (2.7 g, 9.22 mmol) in tBuOH (25 mL) was added KO^{t}Bu (2.07 g, 18.44 mmol) at 0° C. The resulting reaction mixture was stirred at RT for 3 h. The reaction was monitored by TLC and showed that absence of starting material. The reaction mixture was cooled to 0° C. and reaction mixture was poured in ice water and extracted with EtOAc, organic layer was washed with brine and dried over on anhydrous Na₂SO₄ and concentrated under reduced pressure to afford the intermediate G of compound A-24 (2.2 g). LC-MS: 157.1[M-Boc]⁺

### Step-6: Synthesis of tert-butyl 3-(morpholin-2-yl) azetidine-1-carboxylate

To the stirred solution of tert-butyl 3-(5-oxomorpholin-2-yl) azetidine-1-carboxylate (0.9 g, 3.51 mmol) in THE (15 mL) was added BH₃ in THE (1M) (0.91 g, 10.53 mmol) dropwise at 0° C. The resulting reaction mixture was stirred at RT for 6 h. The reaction was monitored by TLC and showed that absence of starting material. The reaction mixture was cooled to 0° C. and reaction mixture was quenched with MeOH slowly and concentrated under reduced pressure to afford the crude material. Crude material was purified by flash chromatography and eluted with 0-20% MeOH in DCM to afford intermediate H of compound A-24 (0.35 g, 41%). LC-MS: 243.2 [M+1]⁺

### Step-7: Synthesis of 3-(2-(1-(tert-butoxy carbonyl) azetidin-3-yl) morpholino)-1-methylcyclobutane-1-carboxylic acid

To the stirred solution of tert-butyl 3-(morpholin-2-yl) azetidine-1-carboxylate (0.15 g, 0.62 mmol) in Toluene (5 mL) was added 1-methyl-3-oxocyclobutane-1-carboxylic acid (0.087 g, 0.68 mmol) and Hantzsch ester (0.2 g, 0.8 mmol) at RT. The resulting reaction mixture was stirred at 110° C. for 16 h. The reaction was monitored by TLC and showed that absence of starting material. The reaction mixture was cooled to RT and poured into water and washed with EtOAc to remove impurities. Then the aqueous layer was concentrated under reduced pressure to afford intermediate J of compound A-24 (0.25 g). LC-MS: 355.2[M+1]⁺

### Step-8: Synthesis of 3-(2-(azetidin-3-yl) morpholino)-1-methylcyclobutane-1-carboxylic acid hydrochloride

To the stirred solution of 3-(2-(1-(tert-butoxy carbonyl) azetidin-3-yl) morpholino)-1-methylcyclobutane-1-carboxylic acid (0.15 g, 0.62 mmol) in water (5 mL) was added con HCl (2.5 ml) at 0° C. The resulting reaction mixture was stirred at RT for 16 h. The reaction was monitored by TLC and showed that absence of starting material. The reaction mixture was cooled to RT and poured into water and washed with EtOAc to remove impurities. Then the aqueous layer was concentrated under reduced pressure to afford the crude compound A-24 (0.2 g). LC-MS: 255.2[M+1]⁺.

### Intermediate I-8 to I-10

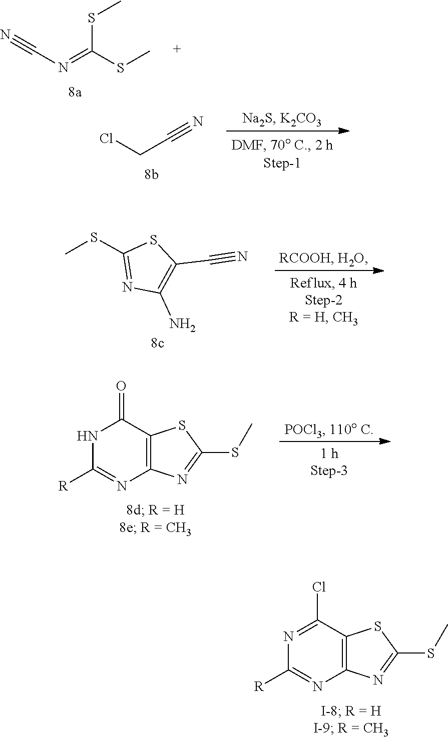

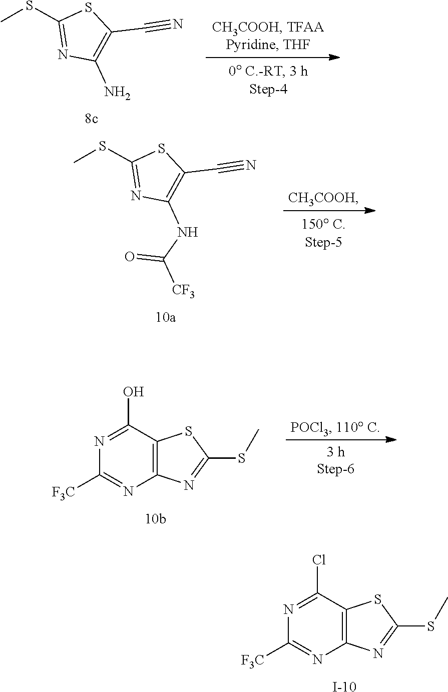

### Step-1: Synthesis of 4-amino-2-(methylthio)thiazole-5-carbonitrile (8c)

To a stirred suspension of Na₂S (5.4 g, 68.4 mmol, 1 eq.) in DMF (80 mL), dimethyl cyanodithioiminocarbonate (10 g, 68.4 mmol, 1 eq.) was added and heated the reaction mixture at 70° C. for 2 hours. Chloroacetonitrile (10.3 g, 136.8 mmol, 2 eq.) was then added slowly at 70° C. to the reaction mixture and stir for 2 hrs at the same temperature followed by potassium carbonate was added to the reaction mixture and stirred for 1 h at the same temperature. After the completion of reaction pour the mixture onto water under good stirring to get solid precipitate, which was filtered and washed with water, dried under vacuum to get tittle compound 8c (7.8 g crude yield). LC-MS: 172.2 [M+H]⁺.

### Step-2 Synthesis of 2-(methylthio)thiazolo[4,5-d]pyrimidin-7(6H)-one (8d)

To a stirred solution of 4-amino-2-(methylthio)thiazole-5-carbonitrile (8c) (1.1 g, 6.42 mmol) in formic acid (4 mL), water (1 drop) was added and heated to reflux for 4 hrs. The progress of the reaction was monitored by TLC. The mixture was allowed to stand at RT and after completion of the reaction was added to ice water to get precipitate, which was collected by filtration, washed with water, dried under vacuum to get compound 8d (0.85 g crude yield). LC-MS: 200.2 [M+H]⁺.

### Step-3: Synthesis of 7-chloro-2-(methylthio)thiazolo[4,5-d]pyrimidine (I-8)

To a stirred solution of 2-(methylthio)thiazolo[4,5-d]pyrimidin-7(6H)-one (8d) (0.1 g, 0.5 mmol) in POCl₃ (1 mL) was heated at 110° C. for 1 hr. The progress of the reaction was monitored by TLC analysis. After completion of the reaction added to the ice water to get precipitate, which was collected by filtration, washed with water, dried under vacuum to get compound I-8 (0.05 g crude yield). LC-MS: 218.7 [M+H]⁺.

The procedure for synthesis of I-9 was same as for I-8.

### Step-4: Synthesis of N-(5-cyano-2-(methylthio)thiazol-4-yl)-2,2,2-trifluoroacetamide (10a)

To a stirred solution of 4-amino-2-(methylthio)thiazole-5-carbonitrile (8c) (2 g, 11.7 mmol, 1 eq.) in THF (20 mL), pyridine (1.03 mL, 12.8 mmol, 1.1 eq.) was added followed by trifluoro acetic anhydride (1.64 mL, 11.7 mmol, 1 eq.) at 0° C. and continued stirring at RT for 3 h. The progress of the reaction was monitored by TLC. After the completion of reaction concentrated under high vacuum and crude was diluted with ice cold water and extracted with CH₂Cl₂ (2 times) and combined organic layer was further washed with brine, dried over anhydrous Na₂SO₄ and concentrated under vacuum to afford the crude 10a (4.8 g crude yield) which was used without further purification. LC-MS: 268.2 [M+H]⁺.

### Step-5: Synthesis of 2-(methylthio)-5-(trifluoromethyl)thiazolo[4,5-d]pyrimidin-7-ol (10b)

AcOH (40 mL) was added to N-(5-cyano-2-(methylthio)thiazol-4-yl)-2,2,2-trifluoroacetamide (10a) (3 g) at 0° C. and continued stirring at 150° C. for 16 h. Progress of the reaction was monitored by TLC. After the completion of reaction, concentrated under high vacuum to afford the crude solid which was triturated with pentane and dried under reduced pressure to get 10b (1.2 g crude yield); LC-MS: 268.2 [M+H]⁺.

### Step-6: synthesis of 2-(methylthio)-5-(trifluoromethyl)thiazolo[4,5-d]pyrimidin-7-ol (I-10)

Phosphorus(V) oxychloride (19.2 mL, 205.8 mmol, 50 eq.) was added to 2-(methylthio)-5-(trifluoromethyl)thiazolo[4,5-d]pyrimidin-7-ol (10b) (1.1 g, 4.1 mmol) at 0° C. and continued stirring at 110° C. for 3 h. The progress of the reaction was monitored by TLC. After completion of the reaction concentrated under high vacuum to afford the crude which was diluted with ice cold water and extracted with Et₂O (2 times) and combined organic layer was further washed with brine, dried over anhydrous Na₂SO₄ and concentrated under vacuum to afford the crude solid which was triturated with pentane and dried under reduced pressure to get I-10 (1.3 g crude yield). LC-MS: 286.7 [M+H]⁺.

### Intermediate I-11: Synthesis of 1-(3,5-dichloropyridin-2-yl)ethan-1-amine

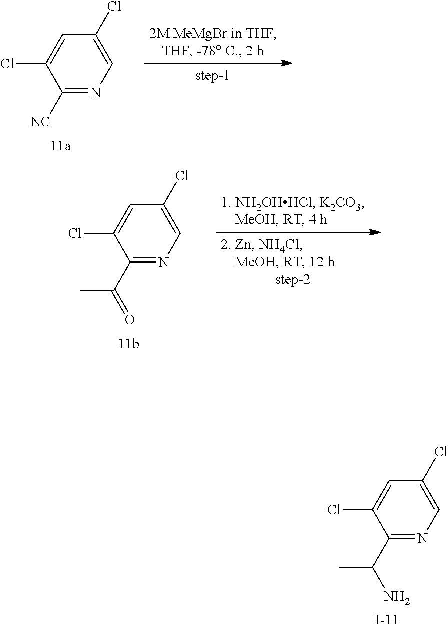

### Step-1: Synthesis of 1-(3,5-dichloropyridin-2-yl)ethan-1-one

To the stirred solution of 3,5-dichloropicolinonitrile (5 g, 28.90 mmol) in dry THF (50 mL) was added 2M Methyl magnesium bromide in THF solution (6.89 g, 57.80 mmol) at −78° C. within 15 min. The resulting reaction mixture continued for 2 h. The reaction was monitored by TLC and showed that absence of starting material. The reaction mixture was quenched with saturated aqueous NH₄Cl solution, extracted with EtOAc, washed with brine, dried over anhydrous Na₂SO₄, organic layer was evaporated to dryness under reduced pressure to afford the crude compound. The crude compound was purified by combi flash using 0-5% ethyl acetate in hexane to get the title compound 11a (3.3 g, 60%). LC-MS: 190.0 [M+H]⁺.

### Step-2: Synthesis of 1-(3,5-dichloropyridin-2-yl)ethan-1-amine

To the stirred solution of 1-(3,5-dichloropyridin-2-yl)ethan-1-one (11a) (1.0 g, 5.26 mmol) in MeOH (20 mL) was added Hydroxylamine hydrochloride (0.55 g, 7.89 mmol) followed by sodium carbonate (0.84 g, 7.89 mmol) at RT. The resulting reaction mixture was heated to 60° C. for 3 h. The reaction was monitored by TLC and showed that absence of starting material. The reaction mixture was cooled to RT and was added NH₄Cl (1.41 g, 53.49 mmol) in water (5 mL) followed by zinc (3.44 g, 52.61 mmol) at RT and continued up to 12 h. After the completion of the reaction, RM was filtered through celite padded sintered funnel, washed the solid with excess of ethyl acetate. Filtrate was evaporated to dryness under reduced pressure to afford the crude compound. The crude compound was purified by combi flash using 0-5% of MeOH in DCM to afford title compound I-11. (0.65 g, 65%). LC-MS: 191.0 [M+H]⁺, 1H-NMR (400 MHz, DMSO-d6) δ 8.65 (d, 1H), 8.28 (d, 1H), 4.52-4.51 (m, 3H), 1.32 (d, 3H).

The intermediate listed below in Table-C was prepared using a similar procedure described in the synthesis of I-11 with appropriate variations in reactants, quantities of reagents and solvents, and reaction conditions. The characterization data of the compounds are summarized herein the below table.

TABLE C

| Int. | Starting | | |
|---|---|---|---|
| No. | material | Intermediate | Spectral data |
| A-25 | 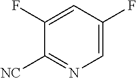 | 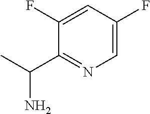 | LC-MS: 159.1 [M + H]⁺ |

### General Synthetic Schemes

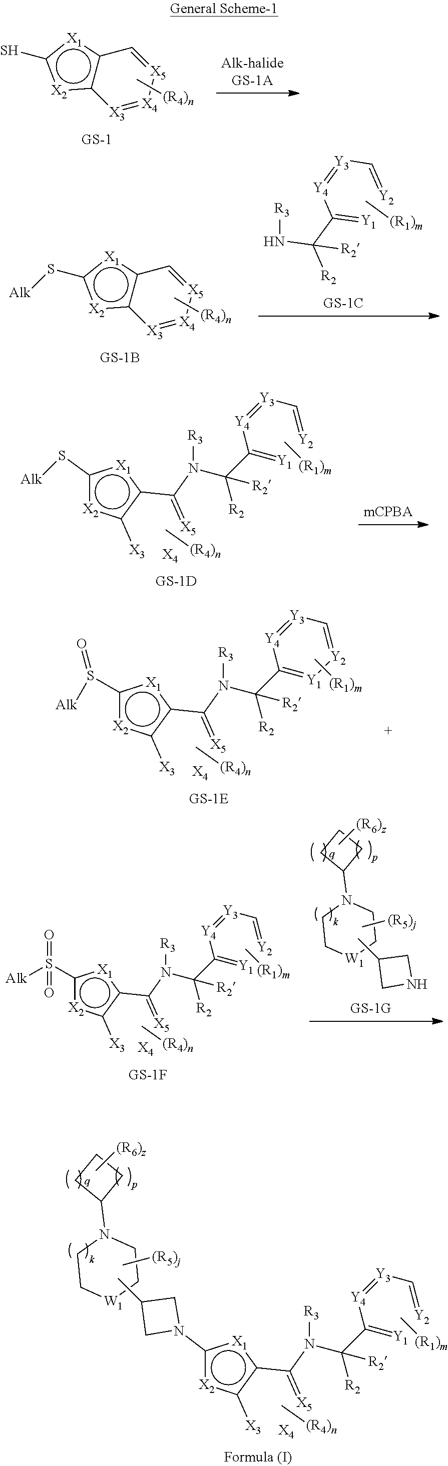

wherein, X₁, X₂ X₃, X₄, X₅, Y₁, Y₂, Y₃, Y₄, R₁, R₂, R₂′, R₃, R₄, R₅, R₆, j, k, m, n, and p are as defined in formula (I).

Some exemplary compounds of the present application may be generally synthesized utilizing the process outlined in General Scheme-1. The commercially available or synthesized GS-1 was reacted with GS-1A (alkyl halide) in presence of suitable reagents and solvents (K₂CO₃, DMF, room temperature) to obtain GS-1B which upon reacting with GS-1C in presence of suitable reagents and solvents (toluene, Xantphos, Pd₂(dba)₃, sodium tertiary butoxide) afforded GS-1D. GS-1D on treatment with mCPBA in presence of suitable reagents and solvents (chloroform) afforded mixture of GS-1E and GS-1F. Treatment of mixture of GS-1E and GS-1F with GS-1G in presence of suitable reagents and solvents (DMF, dioxane, DIPEA) afforded compound of formula (I).

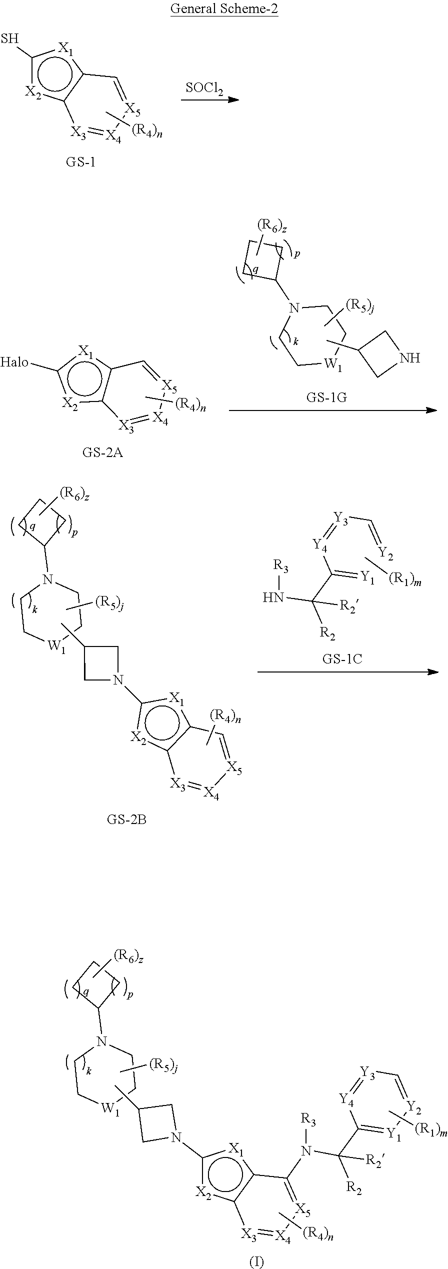

Some exemplary compounds of the present application may be generally synthesized utilizing the process outlined in General Scheme-2. The commercially available or synthesized GS-1 was reacted with suitable reagents and solvents (SOCl₂, DMF) to obtain GS-2A which upon reacting with GS-1G in presence of suitable reagents and solvents (dioxane, DIPEA) afforded GS-2B. GS-2B on treatment with GS-1C in presence of suitable reagents and solvents (CS₂CO₃, X-Phos, Pd₂(dba)₃, 1,2-DME, tert-butanol) afforded compound of formula (I).

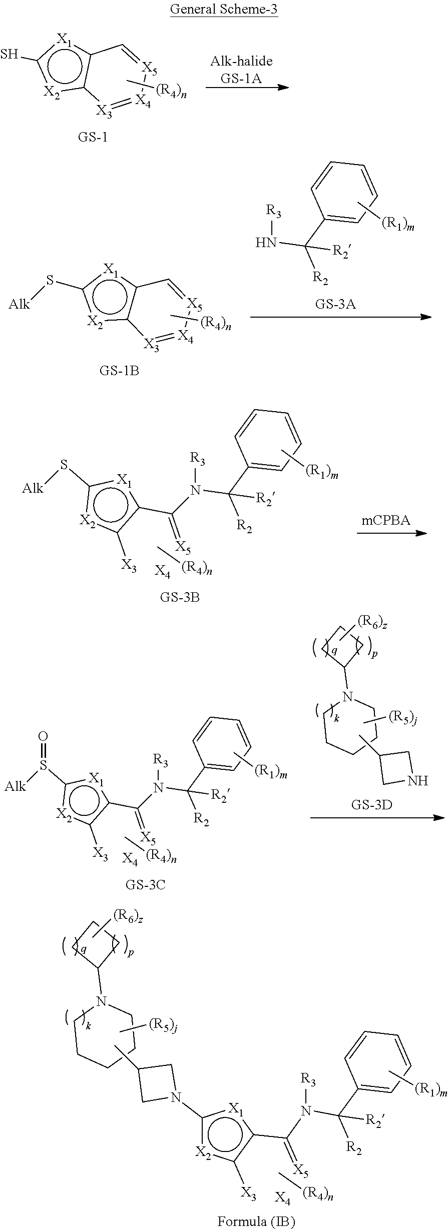

wherein, X₁, X₂ X₃, X₄, X₅, R₁, R₂, R₂′, R₃, R₄, R₅, R₆, j, k, m, n, and p are as defined in formula (I).

Some exemplary compounds of the present application may be generally synthesized utilizing the process outlined in General Scheme-3. The commercially available or synthesized GS-1 was reacted with GS-1A (alkyl halide) in presence of suitable reagents and solvents (K₂CO₃, DMF, room temperature) to obtain GS-1B which upon reacting with GS-3A in presence of suitable reagents and solvents (toluene, Xantphos, Pd₂(dba)₃, sodium tertiary butoxide) afforded GS-3B. GS-3B on treatment with mCPBA in presence of suitable reagents and solvents (chloroform) afforded GS-3C. Treatment of GS-3C with GS-3D in presence of suitable reagents and solvents (DMF, dioxane, DIPEA) afforded compound of formula (IB).

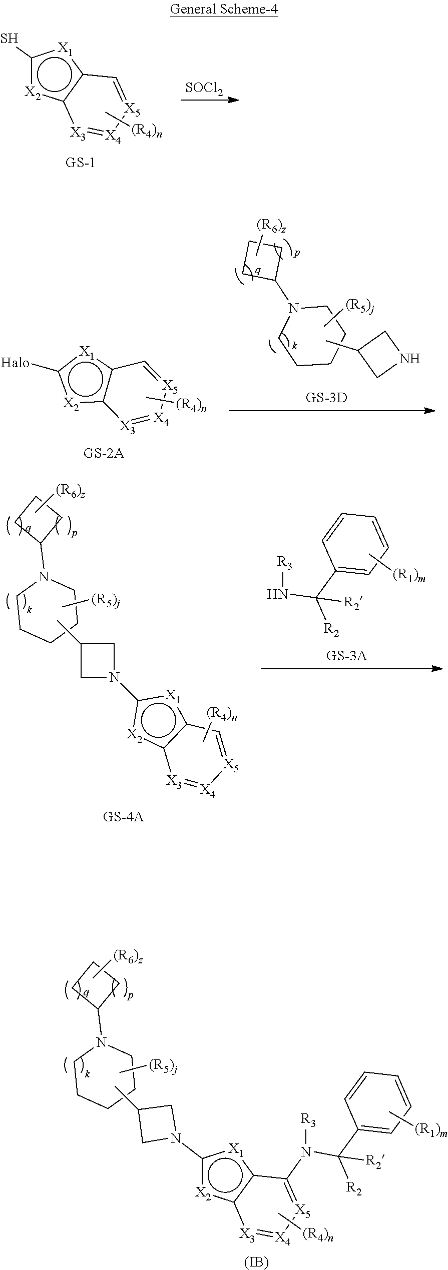

Some exemplary compounds of the present application may be generally synthesized utilizing the process outlined in General Scheme-4. The commercially available or synthesized GS-1 was reacted with suitable reagents and solvents (SOCl₂, DMF) to obtain GS-2A which upon reacting with GS-3D in presence of suitable reagents and solvents (dioxane, DIPEA) afforded GS-4A. GS-4A on treatment with GS-3A in presence of suitable reagents and solvents (CS₂CO₃, X-Phos, Pd₂(dba)₃, 1,2-DME, tert-butanol) afforded compound of formula (IB).

### EXAMPLES

### Example-1: Synthesis of (1R,3r)-3-((R)-3-(1-(6-Chloro-7-(((R)-1-(2,4-dichlorophenyl)ethyl)amino)benzo[d]oxazol-2-yl)azetidin-3-yl)piperidin-1-yl)-1-methylcyclobutane-1-carboxylic acid (Compound-1)

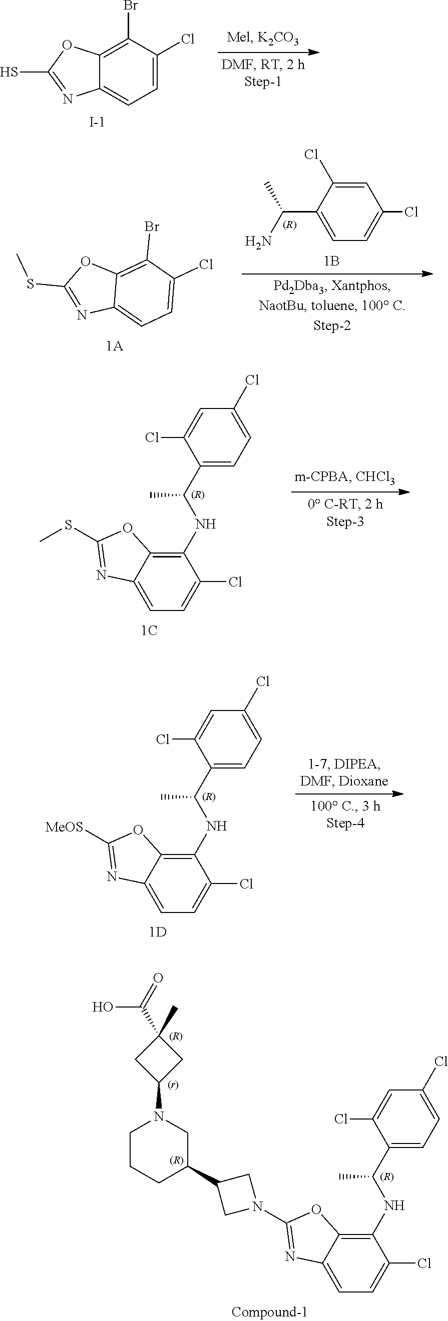

### Step-1: Synthesis of 7-bromo-6-chloro-2-(methylthio)benzo[d]oxazole (1A)

To the stirred solution of 7-bromo-6-chlorobenzo[d]oxazole-2-thiol (2 g, 7.56 mmol) in DMF (20 mL) was added anhydrous K₂CO₃ (2.09 g, 15.12 mmol) at 0° C. and stirred for 10 minutes at the same temperature. Iodomethane (1.61 g, 11.34 mmol) was added at same temperature and the reaction mixture was gradually warmed to RT. The reaction mixture was stirred at RT for 2 h. After the completion of reaction, poured into cold water and extracted with ethyl acetate. The organic portion was washed with water (2 times) and the layers were separated. Organic layer was dried over anhydrous Na₂SO₄, filtered, and concentrated to get crude compound. The crude compound was purified by flash chromatography using 0-10% ethyl acetate in hexane as an eluent to afford the pure title compound 1A (1.5 g, 71.2%). LC-MS: 279.6 [M+H]⁺.

### Step-2: Synthesis of (R)-6-chloro-N-(1-(2,4-dichlorophenyl)ethyl)-2-(methylthio)benzo[d]oxazol-7-amine (1C)

In a sealed tube, 7-bromo-6-chloro-2-(methylthio)benzo[d]oxazole (0.1 g, 0.35 mmol) and (R)-1-(2,4-dichlorophenyl)ethan-1-amine (0.102 g, 0.538 mmol) were taken, dissolved using toluene (6 mL) and stirred at RT. Later, argon gas was purged for 5 minutes, then Xantphos (0.042 g, 0.07 mmol), Pd₂(dba)₃ (0.033 g, 0.03 mmol) and sodium tertiary butoxide (0.069 g, 0.71 mmol) were added sequentially followed by argon purging was continued another for 5 minutes. The resulting reaction mixture was stirred at 100° C. for 1.5 h. After the completion of reaction, the reaction mixture was cooled to RT and concentrated to get the crude compound. The crude compound was purified by flash chromatography on silica gel column using 0-10% ethyl acetate in hexane as an eluent to afford the pure title compound 1C (0.08 g, 71.8%). LC-MS: 388.0 [M+H]⁺.

### Step-3: Synthesis of 6-chloro-N—((R)-1-(2,4-dichlorophenyl)ethyl)-2-(methylsulfinyl)benzo[d]oxazol-7-amine (1D)

To the stirred solution of (R)-6-chloro-N-(1-(2,4-dichlorophenyl)ethyl)-2-(methylthio)benzo[d]oxazol-7-amine (0.07 g, 0.181 mmol) in chloroform (3 mL) was added m-CPBA (0.047 g, 0.27 mmol) at 0° C. and the resulting reaction mixture was stirred at RT for 2 h. After the completion of reaction, the reaction mixture was quenched with saturated aq. NaHCO₃ and extracted with DCM. The organic portion was again washed with water, dried over Na₂SO₄, filtered, and concentrated to get the title compound 1D (0.15 g). LC-MS: 404.9 [M+H]⁺.

### Step-4: Synthesis of (1R,3r)-3-((R)-3-(1-(6-chloro-7-(((R)-1-(2,4-dichlorophenyl)ethyl)amino)benzo[d]oxazol-2-yl)azetidin-3-yl)piperidin-1-yl)-1-methylcyclobutane-1-carboxylic acid (Compound-1)

A mixture of 6-chloro-N—((R)-1-(2,4-dichlorophenyl)ethyl)-2-(methylsulfinyl)benzo[d]oxazol-7-amine (0.13 g, 0.31 mmol), (1R,3r)-3-((R)-3-(azetidin-3-yl)piperidin-1-yl)-1-methylcyclobutane-1-carboxylic acid (0.156 g, 0.62 mmol) in DMF (1 mL), dioxane (1 mL) and was added DIPEA (0.16 g, 1.24 mmol) at 0° C. and the resulting reaction mixture was allowed to reach to RT and then was stirred at 100° C. for 3 h. After the completion of reaction, the reaction mixture was cooled to RT, quenched with saturated NH₄Cl solution, extracted with ethyl acetate and the layers were separated. The organic portion was dried over anhydrous Na₂SO₄, filtered, and concentrated to get crude compound. The crude compound was purified by preparative TLC using 15% methanol in CH₂Cl₂ to afford the title compound 1 (0.09 g, 49.04%). LC-MS: 591.2 [M+H]⁺; ¹H-NMR (400 MHz, CD₃OD) δ 7.47 (d, 1H), 7.37 (d, 1H), 7.27-7.25 (dd, 1H), 7.11 (d, 1H), 6.60 (d, 1H), 5.53-5.46 (m, 1H), 4.29 (t, 1H), 4.20 (t, 1H), 3.99 (t, 1H), 3.83 (t, 1H), 3.29-3.20 (m, 3H), 3.13-3.07 (m, 1H), 2.74-2.69 (m, 2H), 2.68-2.61 (m, 1H), 2.35-2.28 (m, 1H), 2.08-2.02 (m, 1H), 1.98-1.84 (m, 4H), 1.75-1.64 (m, 1H), 1.59 (d, 3H), 1.39 (s, 3H), 1.10-1.03 (m, 1H).

### Example-2: Synthesis of (1R,3r)-3-((R)-3-(1-(7-(((R)-1-(2,4-dichlorophenyl)ethyl)amino)benzo[d]oxazol-2-yl)azetidin-3-yl)piperidin-1-yl)-1-methylcyclobutane-1-carboxylic acid (Compound-2)

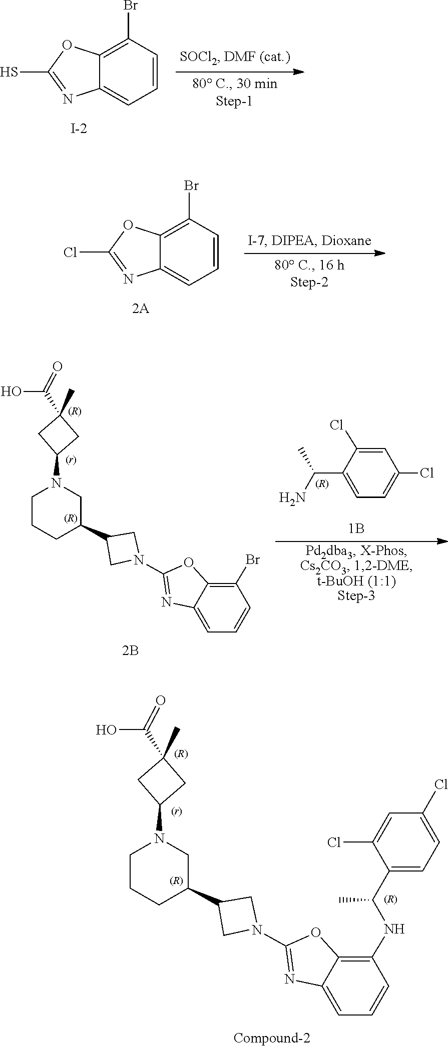

### Step-1: Synthesis of 7-bromo-2-chlorobenzo[d]oxazole (2A)

To the stirred solution of 7-bromobenzo[d]oxazole-2-thiol (1 g, 4.34 mmol) in thionyl chloride (7.2 g, 60.8 mmol), a few drops of DMF were added and the resulting reaction mixture was stirred at 80° C. After 30 minutes, the reaction mixture was cooled to 50° C. and toluene (5 mL) was added, the resultant mixture was concentrated to get residue. The residue was added to the cold water, extracted with toluene and the layers were separated. The organic portion was dried over anhydrous Na₂SO₄, filtered and concentrated. The crude compound was purified by flash chromatography using 10% ethyl acetate in hexane as an eluent to afford the pure title compound 2A (0.53 g, 52.4%). LC-MS: 229.8 [M−H].

### Step-2: Synthesis of (1R, 3r)-3-((R)-3-(1-(7-bromobenzo[d]oxazol-2-yl)azetidin-3-yl)piperidin-1-yl)-1-methylcyclobutane-1-carboxylic acid (2B)

To the stirred solution of (1R,3r)-3-((R)-3-(azetidin-3-yl)piperidin-1-yl)-1-methylcyclobutane-1-carboxylic acid (0.261 g, 1.03 mmol) in dioxane (4 mL), were added DIPEA (0.154 g, 1.19 mmol) and 7-bromo-2-chlorobenzo[d]oxazole (0.185 g, 0.79 mmol) at RT. The resultant mixture was heated to 80° C. and stirred for 16 h. Reaction mixture was then cooled to RT and was concentrated. The residue was washed with ether to get the crude title compound 2B (0.4 g). This was used as such in the next step. LC-MS: 450.3 [M+H+2]⁺.

### Step-3: Synthesis of (1R,3r)-3-((R)-3-(1-(7-(((R)-1-(2,4-dichlorophenyl)ethyl)amino)benzo[d]oxazol-2-yl)azetidin-3-yl)piperidin-1-yl)-1-methylcyclobutane-1-carboxylic acid (Compound-2)

In a sealed tube, (1R,3r)-3-((R)-3-(1-(7-bromobenzo[d]oxazol-2-yl)azetidin-3-yl)piperidin-1-yl)-1-methylcyclobutane-1-carboxylic acid (0.15 g, 0.33 mmol), (R)-1-(2,4-dichlorophenyl)ethan-1-amine (0.083 g, 0.436 mmol) were taken, dissolved using 1:1 ratio of 1.2-DME (3.5 mL), tert-butanol (3.5 mL) and stirred at RT. Later, the reaction mixture was purged with argon gas for 5 minutes, then were added Cs₂CO₃ (0.437 g, 1.34 mmol), X-Phos (0.048 g, 0.1 mmol), Pd₂(dba)₃ (0.031 g, 0.3 mmol) one followed by another and again purged with argon gas for another 5 minutes at RT. The resultant mixture was heated to 110° C. and stirred for 16 h. After the completion of reaction, the reaction mixture was cooled to RT and concentrated to get the crude compound. The crude compound is purified by preparative HPLC-method to get the title compound 2 (0.005 g).

Method: Column: LUNA OMEG PS C-18 (250×21.2 mm) 5μ; eluents: A is 0.05% TFA in water, B is acetonitrile flowrate: 16 mL/min; gradient program: 20% B at 0 min, 30% B at 2 min, 80% B at 8 min. LC-MS: 557.3 [M+H]⁺; ¹H-NMR (400 MHz, CD₃OD) δ 7.48-7.46 (dd, 2H), 7.25-7.22 (dd, 1H), 6.90 (t, 1H), 6.62-6.60 (dd, 1H), 6.22 (d, 1H), 5.17-5.12 (m, 1H), 4.40-4.30 (m, 2H), 4.09-4.01 (m, 2H), 3.85-3.75 (m, 1H), 3.58-3.51 (m, 1H), 3.44-3.36 (m, 1H), 3.28-3.24 (m, 1H), 2.91-2.84 (m, 2H), 2.80-2.70 (m, 2H), 2.58-2.51 (m, 1H), 2.28-2.18 (m, 3H), 2.10-1.98 (m, 3H), 1.53 (d, 3H), 1.47 (s, 3H).

The below compounds were prepared using similar routes as Example-1 and 2 from the respective starting materials given as in Table-D with appropriate variations in coupling methods, reactants, quantities of reagents, and solvents, as required. The characterization data of the compounds are summarized herein the below table.

TABLE D

| | | | Starting | |
|---|---|---|---|---|
| Comp. | | Route of | material | |
| No. | Structure | synthesis | used | Spectral data |
| 3 | 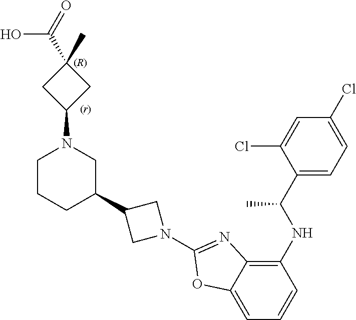 | Ex-2 | A-17 | LC-MS: 557.3 [M + H]⁺; ¹H-NMR (400 MHz, CD₃OD) δ 7.51-7.49 (dd, 2H), 7.29-7.27 (dd, 1H), 6.97 (t, 1H), 6.82 (d, 1H), 6.25 (d, 1H), 5.11-5.09 (m, 1H), 4.57-4.50 (m, 2H), 4.36-4.30 (m, 2H), 3.82-3.70 (m, 1H), 3.55-3.52 (m, 1H), 3.45- 3.42 (m, 1H), 2.90-2.82 (m, 3H), 2.79-2.72 (m, 1H), 2.58-2.52 (m, 1H), 2.40-2.26 (m, 3H), 2.10-2.00 (m, 2H), 1.93-1.85 (m, 1H), 1.60 (d, 3H), 1.48 (s, 3H), 1.29-1.20 (m, 1H). |
| 4 | 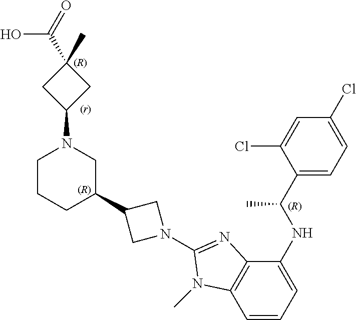 | Ex-2 | I-3 | LC-MS: 570.4 [M + H]⁺; ¹H-NMR (400 MHz, CD₃OD) δ 7.50-7.48 (dd, 2H), 7.25-7.23 (dd, 1H), 7.04 (t, 1H), 6.72 (d, 1H), 6.14 (d, 1H), 5.06-5.04 (m, 1H), 4.72-4.68 (m, 2H), 4.60-4.45 (m, 2H), 3.80-3.72 (m, 1H), 3.71 (s, 3H), 3.54-3.44 (m, 2H), 2.89-2.84 (m, 1H), 2.68 (s, 3H), 2.57-2.39 (m, 4H), 2.10- 1.90 (m, 2H), 1.61 (d, 3H), 1.49 (s, 3H), 1.35-1.32 (m, 2H). |
| 5 | 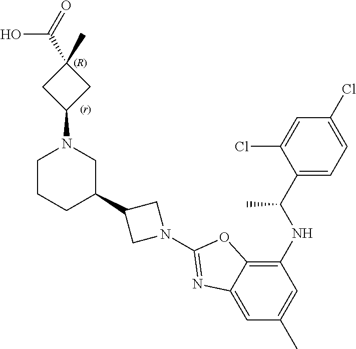 | Ex-1 | A-3 | LC-MS: 571.2 [M + H]⁺; ¹H-NMR (400 MHz, CD₃OD) δ 7.49-7.45 (m, 2H), 7.26-7.24 (dd, 1H), 6.42 (s, 1H), 6.05 (s, 1H), 5.12-5.09 (m, 1H), 4.29-4.24 (m, 2H), 4.02-3.96 (m, 1H), 3.10-2.75 (m, 3H), 2.69- 2.62 (m, 3H), 2.22-2.16 (m, 3H), 2.99-2.72 (m, 5H), 1.68-1.58 (m, 3H), 1.52 (d, 3H), 1.37 (s, 3H), 1.05-0.88 (m, 2H). |
| 6 | 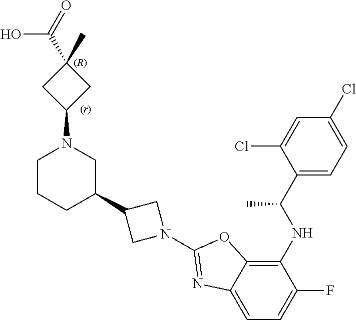 | Ex-1 | A-1 | LC-MS: 575.1 [M + H]⁺; ¹H-NMR (400 MHz, CD₃OD) δ 7.47-7.45 (m, 2H), 7.27-7.24 (dd, 1H), 6.90- 6.85 (m, 1H), 6.53-6.50 (m, 1H), 5.42-5.34 (m, 1H), 4.28-4.22 (t, 1H), 4.20-4.14 (t, 1H), 3.99-3.94 (m, 1H), 3.84-3.77 (m, 1H), 3.08- 2.91 (m, 3H), 2.71-2.55 (m, 3H), 2.09-1.95 (m, 1H), 1.89-1.78 (m, 5H), 1.74-1.62 (m, 2H), 1.57 (d, 3H), 1.37 (s, 3H), 1.35-1.29 (m, 1H) |
| 7 | 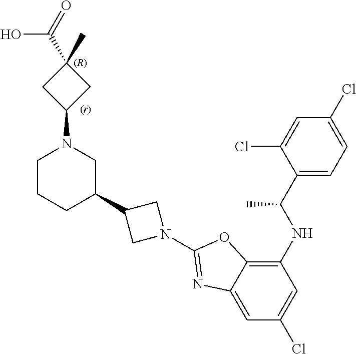 | Ex-1 | A-2 | LC-MS: 591.1 [M + H]⁺; ¹H-NMR (400 MHz, CD₃OD) δ 7.49-7.45 (m, 2H), 7.29-7.26 (dd, 1H), 6.55 (d, 1H), 6.21 (d, 1H), 5.13-5.08 (m, 1H), 4.34-4.29 (m, 2H), 4.07-3.97 (m, 2H), 3.78-3.71 (m, 2H), 3.38- 3.26 (m, 2H), 2.79-2.67 (m, 4H), 2.08-1.88 (m, 6H), 1.54 (d, 3H), 1.40 (s, 3H), 1.37-1.30 (m, 1H). |
| 8 | 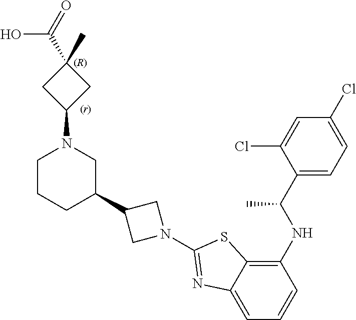 | Ex-2 | I-5 | LC-MS: 573.3 [M + H]⁺; ¹H-NMR (400 MHz, CD₃OD) δ 7.49 (d, 1H), 7.41 (d, 1H), 7.26-7.24 (dd, 1H), 7.20 (t, 1H), 6.81 (d, 1H), 6.19 (d, 1H), 5.06-5.03 (m, 1H), 4.58-4.50 (m, 2H), 4.42-4.37 (m, 2H), 3.82- 3.77 (m, 1H), 3.55-3.50 (m, 1H), 3.48-3.42 (m, 1H), 2.92-2.72 (m, 4H), 2.58-2.52 (m, 1H), 2.46-2.32 (m, 3H), 2.12-1.88 (m, 3H), 1.57 (d, 3H), 1.49 (s, 3H), 1.35-1.25 (m, 1H). |
| 9 | 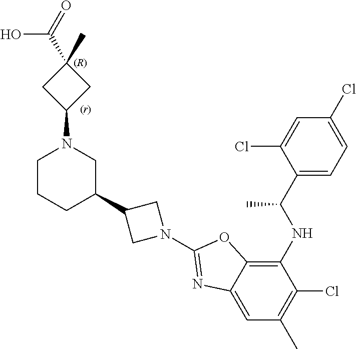 | Ex-1 | A-4 | LC-MS: 605.2 [M + H]⁺; ¹H-NMR (400 MHz, CD₃OD) § 7.46 (d, 1H), 7.38 (d, 1H), 7.27-7.26 (dd, 1H), 6.58 (s, 1H), 5.53-5.42 (m, 1H), 4.24 (t, 1H), 4.16 (t, 1H), 3.99-3.92 (m, 1H), 3.83-3.78 (m, 1H), 3.37 (s, 1H), 3.03-2.95 (m, 1H), 2.91- 2.84 (m, 2H), 2.68-2.55 (m, 4H), 2.36 (s, 3H), 1.85-1.72 (m, 5H), 1.65-1.61 (m, 1H), 1.59 (d, 3H), 1.38 (s, 3H), 1.28-1.32 (m, 1H). |
| 10 | 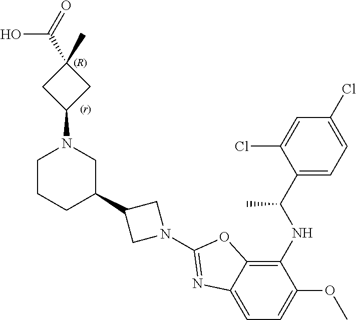 | Ex-1 | A-5 | LC-MS: 587.3 [M + H]⁺; ¹H-NMR (400 MHz, CD₃OD) δ 7.44 (d, 1H), 7.36 (d, 1H), 7.24-7.21 (dd, 1H), 6.79 (d, 1H), 6.58 (d, 1H), 5.40- 5.38 (m, 1H), 4.30-4.18 (m, 2H), 3.98-3.96 (m, 1H), 3.96 (s, 3H), 3.85-3.82 (m, 1H), 3.36-3.30 (m, 1H), 2.80-2.75 (m, 2H), 2.72-2.63 (m, 1H), 2.60-2.52 (m, 1H), 2.38- 2.28 (m, 1H), 2.25-2.16 (m, 2H), 2.05-1.99 (m, 3H), 1.95-1.85 (m, 2H), 1.80-1.72 (m, 1H), 1.55 (d, 3H), 1.41 (s, 3H), 1.22-1.10 (m, 1H). |
| 11 | 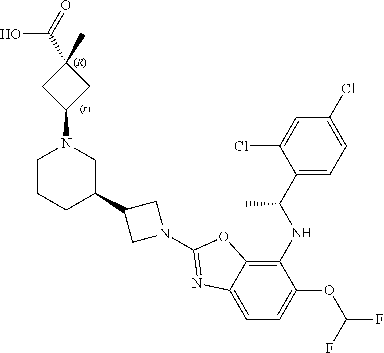 | Ex-1 | A-6 | LC-MS: 623.1 [M + H]⁺; ¹H-NMR (400 MHz, CD₃OD) δ 7.47-7.43 (m, 2H), 7.27-7.25 (dd, 1H), 6.97- 6.95 (m, 1H), 6.78 (s, 1H), 6.59- 6.56 (m, 1H), 5.40-5.38 (m, 1H), 4.26-4.22 (m, 1H), 4.17-4.13 (m, 1H), 3.97-3.93 (m, 1H), 3.80-3.77 (m, 1H), 2.92-2.90 (m, 3H), 2.68- 2.63 (m, 3H), 1.95-1.92 (m, 1H), 1.84-1.79 (m, 4H), 1.68-1.60 (m, 2H), 1.60 (d, 3H), 1.37 (s, 3H), 1.37-1.31 (m, 1H), 0.98-0.92 (m, 1H). |
| 12 | 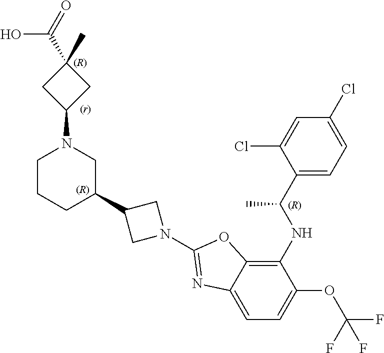 | Ex-1 | A-7 | LC-MS: 641.1 [M + H]⁺; ¹H-NMR (400 MHz, CD₃OD) δ 7.48 (d, 1H), 7.45-7.43 (m, 1H), 7.29-7.26 (dd, 1H), 7.05-7.02 (m, 1H), 6.60 (d, 1H), 5.40-5.38 (m, 1H), 4.26-4.22 (m, 1H), 4.16-4.12 (m, 1H), 3.98- 3.92 (m, 1H), 3.80-3.72 (m, 1H), 2.94-2.87 (m, 1H), 2.85-2.78 (m, 1H), 2.72-2.70 (m, 1H), 2.64-2.59 (m, 3H), 2.05-2.01 (m, 1H), 1.76- 1.69 (m, 6H), 1.55 (d, 3H), 1.48- 1.40 (m, 1H), 1.39 (s, 3H), 1.35- 1.30 (m, 1H). |
| 13 | 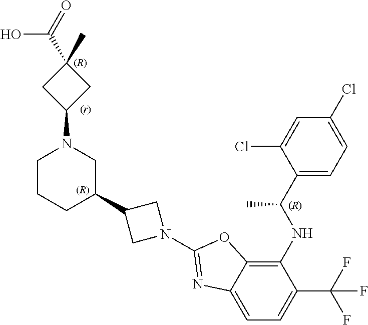 | Ex-1 | A-8 | LC-MS: 625.3 [M + H]⁺; ¹H-NMR (400 MHz, CD₃OD) δ 7.50-7.46 (m, 2H), 7.35-7.28 (m, 2H), 6.69 (d, 1H), 5.51-5.43 (m, 1H), 4.25 (t, 1H), 4.15 (t, 1H), 3.99-3.96 (m, 1H), 3.81-3.77 (m, 1H), 2.98-2.92 (m, 1H), 2.88-2.76 (m, 2H), 2.66- 2.61 (m, 3H), 1.91 (s, 3H), 1.85- 1.72 (m, 6H), 1.65-1.52 (m, 6H). |
| 14 | 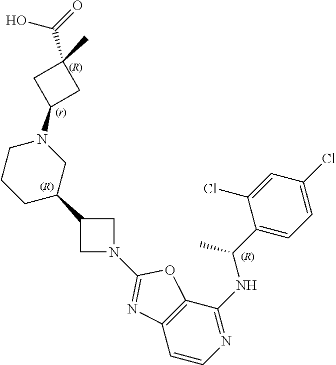 | Ex-1 | A-9 | LC-MS: 558.1 [M + H]⁺; ¹H-NMR (400 MHz, CD₃OD) δ 7.72 (d, 1H), 7.49-7.45 (m, 2H), 7.25 (dd, 1H), 6.64 (d, 1H), 5.53-5.49 (m, 1H), 4.38-4.30 (m, 2H), 4.10-3.99 (m, 2H), 3.06-2.99 (m, 3H), 2.70-2.65 (m, 3H), 2.15-2.08 (m, 1H), 1.93- 1.81 (m, 7H), 1.74-1.60 (m, 1H), 1.58 (d, 3H), 1.38 (s, 3H). |
| 15 | 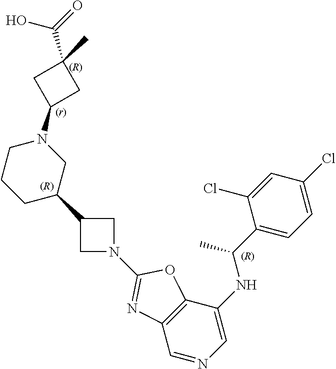 | Ex-1 | A-10 | LC-MS: 558.1 [M + H]⁺; ¹H-NMR (400 MHz, DMSO-d₆) δ 7.84 (s, 1H), 7.59 (s, 2H), 7.51 (d, 1H), 7.38-7.36 (dd, 1H), 6.65 (d, 1H), 5.12-5.08 (m, 1H), 4.20-4.13 (m, 2H), 3.96-3.92 (m, 1H), 3.86-3.80 (m, 1H), 3.43-3.40 (m, 1H), 3.28- 3.25 (m, 1H), 2.36-2.30 (m, 2H), 1.69-1.56 (m, 5H), 1.47 (d, 3H), 1.44-1.32 (m, 6H), 1.12 (s, 3H). |
| 16 | 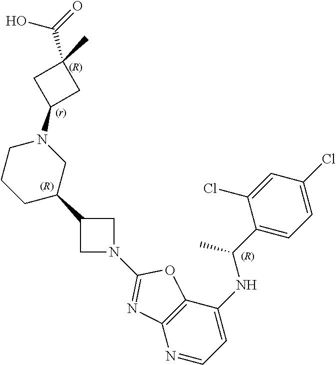 | Ex-1 | A-11 | LC-MS: 558.3 [M + H]⁺; ¹H-NMR (400 MHz, CD₃OD) δ 7.73 (d, 1H), 7.60 (s, 1H), 7.44 (d, 1H), 7.37- 7.35 (dd, 1H), 6.70 (brs, 1H), 5.51 (brs, 1H), 4.52-4.42 (brm, 1H), 4.38-4.32 (brs, 1H), 4.30-4.22 (brs, 1H), 4.12-4.05 (brs, 1H), 3.84-3.72 (m, 1H), 3.51 (brd, 1H), 3.37-3.32 (m, 1H), 2.87-2.72 (m, 4H), 2.52- 2.42 (m, 3H), 2.28-2.20 (brs, 1H), 2.12-2.04 (m, 1H), 1.98-1.89 (m, 2H), 1.64 (d, 3H), 1.48 (s, 3H), 1.28-1.16 (m, 1H). |
| 17 | 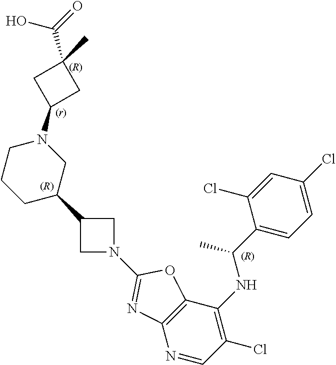 | Ex-1 | A-12 | LC-MS: 592.1 [M + H]⁺; ¹H-NMR (400 MHz, CD₃OD) δ 7.89 (s, 1H), 7.53 (d, 1H), 7.39 (d, 1H), 7.32- 7.30 (dd, 1H), 5.53-5.52 (m, 1H), 4.31-4.27 (m, 1H), 4.20-4.16 (m, 1H), 4.02-3.99 (m, 1H), 3.84-3.80 (m, 1H), 2.89-2.85 (m, 1H), 2.80- 2.73 (m, 1H), 2.73-2.69 (m, 1H), 2.63-2.59 (m, 3H), 1.80-1.70 (m, 6H), 1.66 (d, 3H), 1.62-1.54 (m, 1H), 1.48-1.42 (m, 1H), 1.35 (s, 3H), 0.95-0.88 (m, 1H). |
| 18 | 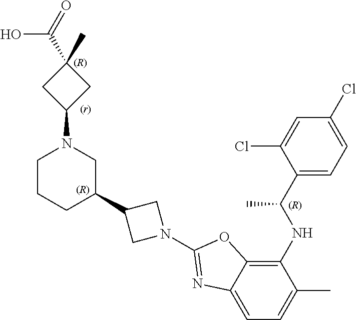 | Ex-1 | A-13 | LC-MS: 571.2 [M + H]⁺; ¹H-NMR (400 MHz, CD₃OD) δ 7.55-7.53 (m, 1H), 7.41 (d, 1H), 7.30-7.27 (m, 1H), 7.14-7.12 (dd, 1H), 6.59 (d, 1H), 5.46-5.41 (m, 1H), 4.60- 4.52 (m, 1H), 4.48-4.35 (m, 2H), 4.22-4.12 (m, 1H), 3.80-3.76 (m, 1H), 3.58-3.50 (m, 1H), 3.44-3.34 (m, 1H), 2.86-2.80 (m, 3H), 2.78- 2.74 (m, 1H), 2.58-2.42 (m, 2H), 2.40-2.36 (m, 4H), 2.34-2.28 (m, 1H), 2.12-2.05 (m, 1H), 2.02-1.90 (m, 2H), 1.62 (d, 3H), 1.49 (s, 3H), 1.30-1.15 (m, 1H). |
| 19 | 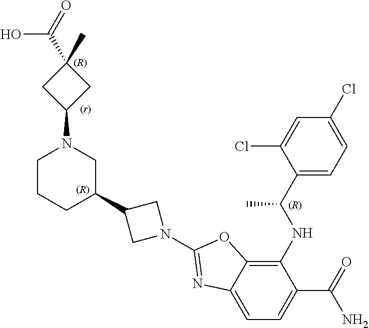 | Ex-1 | A-14 | LC-MS: 600.1 [M + H]⁺; ¹H-NMR (400 MHz, CD₃OD) δ 7.53 (d, 1H), 7.47 (d, 1H), 7.43 (d, 1H), 7.26- 7.23 (dd, 1H), 6.58 (d, 1H), 5.49- 5.46 (m, 1H), 4.32-4.29 (m, 1H), 4.24-4.19 (m, 1H), 4.03-3.99 (m, 1H), 3.85-3.82 (m, 1H), 3.20-3.16 (m, 2H), 3.08-3.04 (m, 1H), 2.73- 2.62 (m, 3H), 2.28-2.22 (m, 1H), 1.95-1.84 (m, 6H), 1.76-1.64 (m, 1H), 1.56 (d, 3H), 1.38 (s, 3H), 1.36-1.32 (m, 1H). |
| 20 | 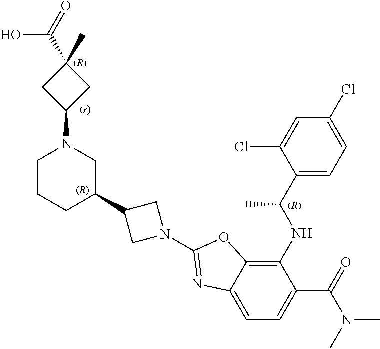 | Ex-1 | A-15 | LC-MS: 628.2 [M + H]⁺; ¹H-NMR (400 MHz, CD₃OD) δ 7.51 (d, 1H), 7.46 (d, 1H), 7.27-7.24 (dd, 1H), 7.03-7.00 (d, 1H), 6.66 (d, 1H), 5.43-5.38 (m, 1H), 4.32-4.27 (m, 1H), 4.24-4.20 (m, 1H), 4.02-3.99 (m, 1H), 3.87-3.84 (m, 1H), 3.14- 3.04 (brm, 8H), 3.02-2.99 (m, 1H), 2.71-2.63 (m, 3H), 2.16-2.08 (m, 1H), 1.92-1.82 (m, 6H), 1.70-1.64 (m, 1H), 1.54 (d, 3H), 1.38 (s, 3H), 1.08-1.00 (m, 1H). |
| 21 | 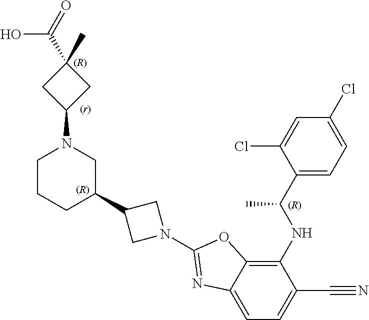 | Ex-1 | A-16 | LC-MS: 582.2 [M + H]⁺; ¹H-NMR (400 MHz, CD₃OD) δ 7.51 (d, 1H), 7.46 (d, 1H), 7.32-7.29 (m, 2H), 6.68 (d, 1H), 5.51-5.49 (m, 1H), 4.34-4.30 (m, 1H), 4.24-4.20 (m, 1H), 4.04-4.00 (m, 1H), 3.86-3.83 (m, 1H), 3.22-3.20 (m, 1H), 3.08- 3.02 (m, 1H), 2.72-2.64 (m, 3H), 2.23-2.18 (m, 1H), 1.94-1.87 (m, 6H), 1.78-1.65 (m, 1H), 1.63 (d, 3H), 1.38 (s, 3H), 1.36-1.30 (m, 1H), 1.08-1.04 (m, 1H). |
| 22 | 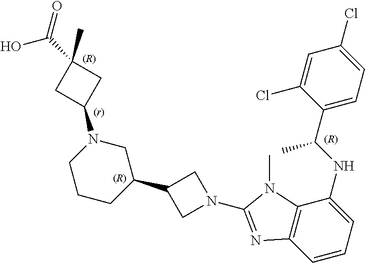 | Ex-2 | I-4 | LC-MS: 570.2 [M + H]⁺; ¹H-NMR (400 MHz, CD₃OD) δ 7.60 (d, 1H), 7.50 (d, 1H), 7.28-7.25 (dd, 1H), 7.04-7.00 (m, 1H), 6.82-6.79 (dd, 1H), 6.29 (d, 1H), 4.98-4.92 (m, 2H), 4.67-4.60 (m, 2H), 4.52-4.46 (m, 1H), 4.44-4.39 (m, 1H), 4.09 (s, 3H), 3.84-3.74 (m, 1H), 3.58- 3.52 (m, 1H), 3,48-3.42 (m, 1H), 2.87-2.76 (m, 4H), 2.55-2.38 (m, 4H), 2.05-1.96 (m, 3H), 1.60 (d, 3H), 1.49 (s, 3H). |
| 23 | 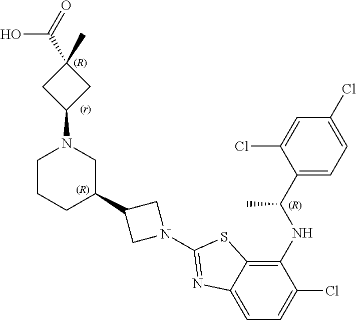 | Ex-1 | A-18 | LC-MS: 607.1 [M + H]⁺; ¹H-NMR (400 MHz, CD₃OD) δ 7.43-7.41 (m, 2H), 7.28-7.25 (m, 2H), 6.94 (d, 1H), 5.35-5.30 (m, 1H), 4.25- 4.16 (m, 2H), 3.98-3.96 (m, 2H), 3.59-3.55 (m, 1H), 3.42-3.35 (m, 1H), 3.28-3.25 (m, 1H), 2.81-2.76 (m, 2H), 2.68-2.59 (m, 2H), 2.36- 2.30 (m, 1H), 2.17-1.91 (m, 5H), 1.78-1.72 (m, 1H), 1.60 (d, 3H), 1.42 (s, 3H), 1.14-1.10 (m, 1H). |
| 24 | 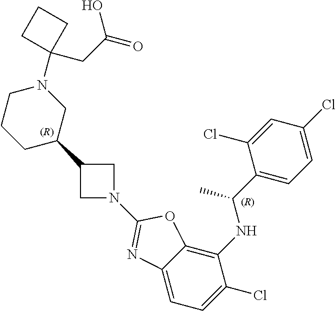 | Ex-1 | I-1 & A-23 | LC-MS: 591.1 [M + H]⁺; ¹H-NMR (400 MHz, CD₃OD) δ 7.48 (d, 1H), 7.38 (d, 1H), 7.27-7.25 (dd, 1H), 7.12 (d, 1H), 6.61(d, 1H), 5.52- 5.46 (m, 1H), 4.33-4.22 (m, 2H), 4.07-4.05 (m, 1H), 3.91-3.88 (m, 1H), 3.52-3.36 (m, 1H), 2.96-2.88 (m, 1H), 2.86-2.82 (m, 2H), 2.80- 2.72 (m, 1H), 2.68-2.62 (m, 3H), 2.24-2.06 (m, 2H), 1.93-1.90 (m, 5H), 1.84-1.72 (m, 1H), 1.60 (d, 3H), 1.41-1.37 (m, 1H), 1.35-1.31 (m, 1H). |
| 25 | 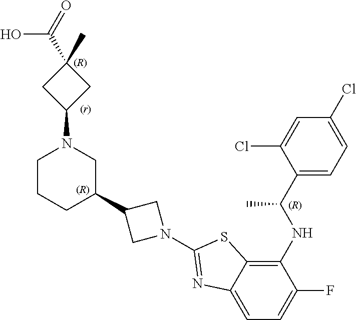 | Ex-1 | A-19 | LC-MS: 591.1 [M + H]⁺; ¹H-NMR (400 MHz, CD₃OD) δ 7.49 (d, 1H), 7.40 (d, 1H), 7.27-7.24 (dd, 1H), 7.03-6.98 (m, 1H), 6.89-6.86 (m, 1H), 5.29-5.25 (m, 1H), 4.26-4.18 (m, 2H), 3.97-3.93 (m, 2H), 3.47- 3.41 (m, 1H), 3.26-3.18 (m, 1H), 2.77-2.67 (m, 4H), 2.54-2.43 (m, 1H), 2.28-2.19 (m, 1H), 2.20-1.90 (m, 5H), 1.78-1.68 (m, 1H), 1.57 (d, 3H), 1.40 (s, 3H), 1.18-1.09 (m, 1H). |
| 26 | 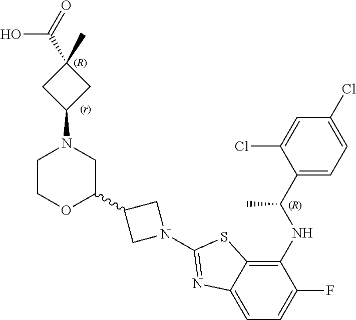 | Ex-1 | A-19 & A-24 | LC-MS: 593.1 [M + H]⁺; ¹H-NMR (400 MHz, CD₃OD) δ 7.48 (d, 1H), 7.40 (d, 1H), 7.26-7.24 (dd, 1H), 7.01-6.95 (m, 1H), 6.88-6.84 (m, 1H), 5.28-5.26 (m, 1H), 4.19-4.10 (m, 3H), 4.05-4.01 (m, 1H), 3.96- 3.93 (m, 1H), 3.71-3.66 (m, 2H), 2.98-2.92 (m, 1H), 2.80-2.74 (m, 3H), 2.68-2.60 (m, 2H), 2.06- 1.95 (m, 1H), 1.82-1.75 (m, 2H), 1.68-1.62 (m, 1H), 1.56 (d, 3H), 1.37 (s, 3H). |
| | (chiral separation method: Chiral pak-IG Dimension 250 × | | | |
| | 21.2 mm, 5-micron, Flow Rate 15.0 ml per minute, Mobile | | | |
| | Phase-A: n-HEXANE, Mobile Phase-B: Ethanol | | | |
| 27 | 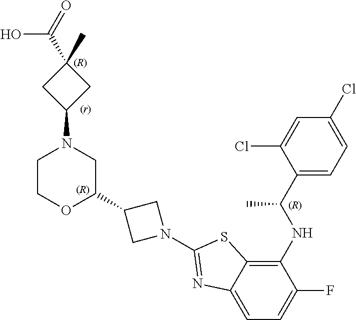 | Ex-1 | A-19 & A-24 | LC-MS: 593.1 [M + H]⁺; ¹H-NMR (400 MHz, CD₃OD) δ 7.49 (d, 1H), 7.40 (d, 1H), 7.27-7.24 (dd, 1H), 7.01-6.96 (m, 1H), 6.88-6.85 (m, 1H), 5.30-5.25 (m, 1H), 4.22-4.17 (m, 1H), 4.13-4.11 (m, 2H), 4.04- 3.98 (m, 2H), 3.72-3.67 (m, 2H), 2.98-2.86 (m, 2H), 2.84-2.77 (m, 2H), 2.63-2.58 (m, 2H), 2.09- 2.01 (m, 1H), 1.89-1.85 (m, 2H), 1.74-1.66 (m, 1H), 1.56 (d, 3H), 1.39 (s, 3H). |
| | (Isomer-1 of compound 26) | | | |
| 28 | 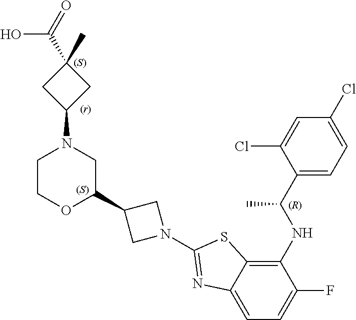 | Ex-1 | A-19 & A-24 | LC-MS: 593.15 [M + H]⁺; ¹H-NMR (400 MHz, CD₃OD) δ 7.48 (d, 1H), 7.40 (d, 1H), 7.27-7.24 (dd, 1H), 7.01-6.96 (m, 1H), 6.88-6.85 (m, 1H), 5.30-5.27 (m, 1H), 4.19-4.10 (m, 3H), 4.06-4.02 (m, 1H), 4.00- 3.95 (m, 1H), 3.72-3.66 (m, 2H), 2.96-2.90 (m, 2H), 2.84-2.78 (m, 2H), 2.64-2.58 (m, 2H), 2.12- 2.04 (m, 1H), 1.91-1.84 (m, 2H), 1.75-1.68 (m, 1H), 1.56 (d, 3H), 1.39 (s, 3H). |
| | (Isomer-2 of compound 26) | | | |
| 29 | 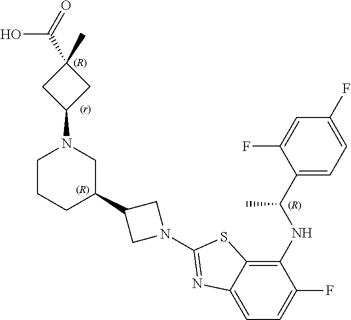 | Ex-1 | A-19 | LC-MS: 559.2 [M + H]⁺; ¹H-NMR (400 MHz, CD₃OD) δ 7.45 (m, 1H), 7.21-7.16 (m, 1H), 6.93-6.85 (m, 3H), 5.18-5.12 (m, 1H), 4.50- 4.43 (m, 2H), 4.36-4.30 (m, 2H), 3.84-3.72 (m, 1H), 3.54-3.50 (m, 1H), 3.46-3.38 (m, 1H), 2.88-2.83 (m, 3H), 2.78-2.71 (m, 1H), 2.56- 2.30 (m, 4H), 2.10-1.85 (m, 3H), 1.62 (d, 3H), 1.48 (s, 3H), 1.25- 1.19 (m, 1H). |
| 30 | 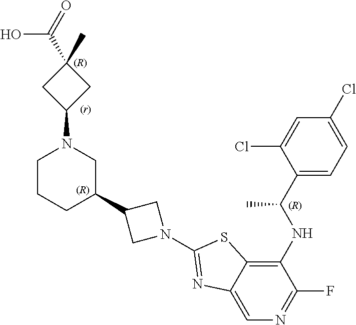 | Ex-1 | A-20 | LC-MS: 592.1 [M + H]⁺; ¹H-NMR (400 MHz, CD₃OD) δ 7.58 (d, 1H), 7.49 (d, 1H), 7.46 (d, 1H), 7.32- 7.30 (m, 1H), 5.30-5.25 (m, 1H), 4.46-4.36 (m, 2H), 4.32-4.20 (m, 2H), 3.79-3.75 (m, 1H), 3.55-3.49 (m, 1H), 2.85-2.80 (m, 3H), 2.78- 2.70 (m, 1H), 2.51-2.34 (m, 4H), 2.08-1.88 (m, 4H), 1.62 (d, 3H), 1.48 (s, 3H), 1.27-1.18 (m, 1H). |
| 31 | 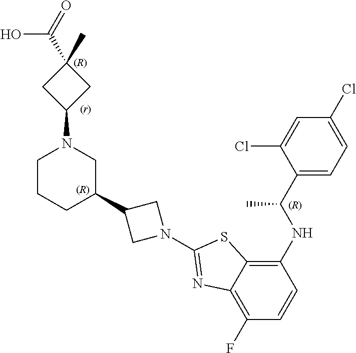 | Ex-1 | I-6 | LC-MS: 591.3 [M + H]⁺; ¹H-NMR (400 MHz, CD₃OD) δ 7.48-7.46 (m, 2H), 7.27-7.25 (dd, 1H), 7.15 (d, 1H), 6.51 (d, 1H), 5.01-4.85 (m, 2H), 4.19-4.11 (m, 2H), 3.90-3.86 (m, 2H), 3.24-3.16 (m, 2H), 3.14- 3.06 (m, 1H), 2.72-2.67 (m, 2H), 2.64-2.54 (m, 1H), 2.34-2.22 (m, 1H), 1.97-1.87 (m, 6H), 1.52 (d, 3H), 1.38 (s, 3H), 1.08-1.02 (m, 1H). |

### Example-3: Synthesis of (1R,3r)-3-((R)-3-(1-(7-(((R)-1-(2,4-Dichlorophenyl)ethyl)amino)thiazolo[4,5-d]pyrimidin-2-yl)azetidin-3-yl)piperidin-1-yl)-1-methylcyclobutane-1-carboxylic acid (Compound-32)

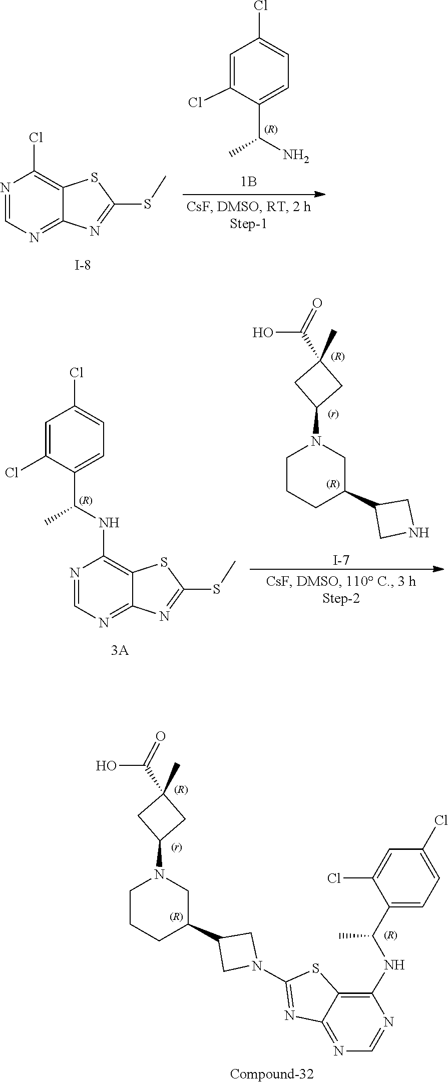

### Step-1: (R)—N-(1-(2,4-dichlorophenyl)ethyl)-2-(methylthio)thiazolo[4,5-d]pyrimidin-7-amine (3A)

To the suspension of 7-chloro-2-(methylthio)thiazolo[4,5-d]pyrimidine (I-8) (0.6 g, 2.75 mmol, 1 eq.) and 1B(0.78 g, 4.1 mmol, 1.5 eq.) in DMSO (12 mL) was added CsF (1.25 g, 8.26 mmol, 3 eq.) and stirred the reaction mixture at RT for 2 hrs. The progress of the reaction was monitored by TLC. After the completion of reaction, the mixture was dissolved in ice-water and extracted with EtOAc (3 times) and the combined organic layer was dried over anhydrous Na₂SO₄, concentrated under high vacuum to get crude which was purified by flash chromatography on silica gel column using 30-40% EtOAc in hexane as an eluent to afford pure tittle compound 3A (0.65 g, 63% yield). LC-MS: 372.3 [M+H]⁺.

### Step-2: Synthesis of (1R,3r)-3-((R)-3-(1-(7-(((R)-1-(2,4-dichlorophenyl)ethyl)amino)thiazolo[4,5-d]pyrimidin-2-yl)azetidin-3-yl)piperidin-1-yl)-1-methylcyclobutane-1-carboxylic acid (Compound-32)

To the stirred solution of 3A (0.05 g, 0.13 mmol, 1 eq.) and (1R,3r)-3-((R)-3-(azetidin-3-yl)piperidin-1-yl)-1-methylcyclobutane-1-carboxylic acid (I-7) (0.06 g, 0.27 mmol, 2 eq.) in DMSO (3 mL) was added cesium fluoride (0.06 g, 0.4 mmol, 3 eq.) at RT and heated at 100° C. for 3 hrs. The progress of the reaction was monitored by TLC. After completion of the reaction, ice water was added to the reaction mixture to get crude solid which was purified by preparative TLC using 15% methanol in CH₂Cl₂ to afford the title compound (0.03 g, 39% yield). LC-MS: 575.1 [M+H]⁺; 1H-NMR (400 MHz, CD3OD) δ 8.24 (s, 1H), 7.47 (d, 1H), 7.42 (d, 1H), 7.29-7.27 (dd, 1H), 5.71-5.66 (m, 1H), 4.35-4.31 (m, 2H), 4.12-4.08 (m, 2H), 3.84-3.72 (m, 1H), 3.55-3.46 (m, 1H), 3.44-3.36 (m, 1H), 2.89-2.74 (m, 4H), 2.58-2.48 (m, 1H), 2.28-2.19 (m, 3H), 2.10-1.93 (m, 2H), 1.90-1.72 (i, 1H), 1.58 (d, 3H), 1.44 (s, 3H), 1.32-1.18 (m, 1H).

The below compounds were prepared using similar routes as Example-3 from the respective starting materials given as in Table-E with appropriate variations in coupling methods, reactants, quantities of reagents, and solvents, as required. The characterization data of the compounds are summarized herein the below table.

TABLE-E

| | | | Starting | |
|---|---|---|---|---|
| Comp. | | Route of | material | |
| No. | Structure | synthesis | used | Spectral data |
| 33 | 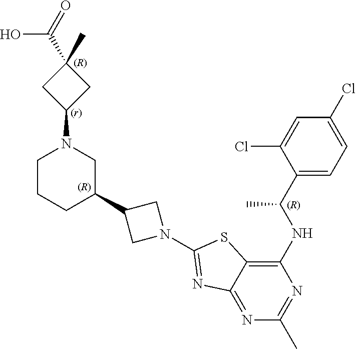 | Ex-3 | I-9 | LC-MS: 589.2 [M + H]⁺; ¹H-NMR (400 MHz, CD₃OD) δ 7.50 (d, 1H), 7.45 (d, 1H), 7.35-7.32 (dd, 1H), 5.88-5.76 (bs, 1H), 4.46-4.38 (brs, 2H), 4.28-4.16 (brs, 2H), 3.79-3.75 (m, 1H), 3.53-3.50 (m, 1H), 3.44-3.39 (m, 1H), 2.85-2.74 (m, 3H), 2.68 (s, 3H), 2.55-2.48 (m, 1H), 2.46-2.20 (m, 3H), 2.10-1.85 (m, 3H), 1.62 (d, 3H), 1.48 (s, 3H), 1.36-1.28 (m, 2H). |
| 34 | 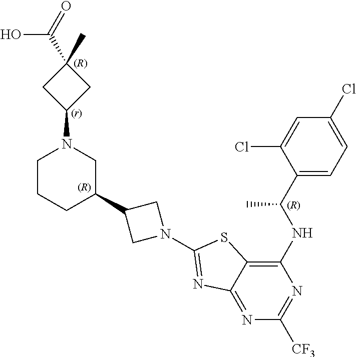 | Ex-3 | I-10 | LC-MS: 643.1 [M + H]⁺; ¹H-NMR (400 MHz, CD₃OD) δ 7.45-7.41 (m, 2H), 7.29-7.26 (dd, 1H), 5.73-5.68 (m, 1H), 4.37-4.30 (m, 2H), 4.11-4.05 (m, 2H), 3.28-3.15 (m, 2H), 2.76-2.70 (m, 3H), 2.42-2.36 (m, 1H), 2.22-2.12 (m, 1H), 2.10-2.00 (m, 2H), 1.99-1.91 (m, 4H), 1.78-1.66 (m, 1H), 1.58 (d, 3H), 1.39 (s, 3H), 1.18-1.06 (m, 1H). |

### Example-4: Synthesis of (1R,3r)-3-((3R)-3-(1-(7-((1-(3,5-dichloropyridin-2-yl)ethyl)amino)-6-fluorobenzo[d]thiazol-2-yl)azetidin-3-yl)piperidin-1-yl)-1-methylcyclobutane-1-carboxylic acid (Compound-35), (1R,3r)-3-((R)-3-(1-(7-(((R)-1-(3,5-dichloropyridin-2-yl)ethyl)amino)-6-fluorobenzo[d]thiazol-2-yl)azetidin-3-yl)piperidin-1-yl)-1-methylcyclobutane-1-carboxylic acid (Compound-36), and (1R,3r)-3-((R)-3-(1-(7-(((S)-1-(3,5-dichloropyridin-2-yl)ethyl)amino)-6-fluorobenzo[d]thiazol-2-yl)azetidin-3-yl)piperidin-1-yl)-1-methylcyclobutane-1-carboxylic acid (Compound-37)

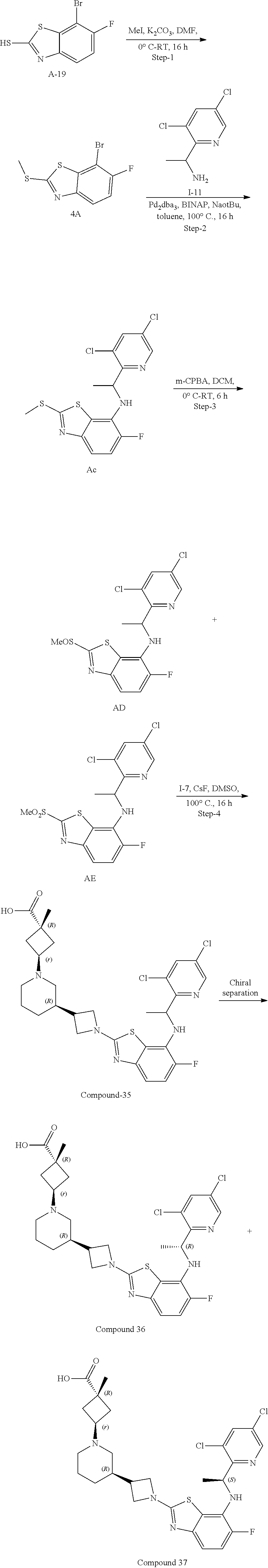

### Step-1: Synthesis of 7-bromo-6-fluoro-2-(methylthio)benzo[d]thiazole (4A)

To the stirred solution of A-19 (12 g, 45.43 mmol) in DMF (120 mL) was added anhydrous K₂CO₃ (12.56 g, 90.86 mmol) at 0° C. and stirred for 10 minutes at the same temperature. Later, Iodomethane (9.67 g, 68.14 mmol) was added at same temperature and the reaction mixture was gradually warmed to RT. Then the reaction mixture was stirred at RT for 16 h. After the completion of reaction, the reaction mixture was poured into cold water and obtained precipitate was filtered, washed with excess of ice-cold water, dried under line vacuum to get crude compound 4A (10 g). LC-MS: 277.9 [M+H]⁺.

### Step-2: Synthesis of N-(1-(3,5-dichloropyridin-2-yl)ethyl)-6-fluoro-2-(methylthio)benzo[d]thiazol-7-amine (4C)

In a sealed tube, 7-bromo-6-fluoro-2-(methylthio)benzo[d]thiazole (4A) (0.5 g, 1.80 mmol) and I-11 (0.51 g, 2.70 mmol) were taken, dissolved using toluene (15 mL) and stirred at RT. Later, argon gas was purged for 5 minutes, then BINAP (0.22 g, 0.36 mmol), Pd₂(dba)₃ (0.29 g, 0.36 mmol) and sodium tertiary butoxide (0.35 g, 3.60 mmol) were added one followed by argon purging continued another 5 minutes. The resulting reaction mixture was stirred at 100° C. for 16 h. After the completion of reaction, the reaction mixture was cooled to RT and concentrated to get the crude compound. The crude compound was purified by flash chromatography on silica gel column using 0-20% ethyl acetate in hexane as an eluent to afford the pure title compound 4C (0.22 g, 31%). LC-MS: 388.0 [M+H]⁺.

### Step-3: Synthesis of N-(1-(3,5-dichloropyridin-2-yl)ethyl)-6-fluoro-2-(methylsulfinyl)benzo[d]thiazol-7-amine (4D) and N-(1-(3,5-dichloropyridin-2-yl)ethyl)-6-fluoro-2-(methylsulfonyl)benzo[d]thiazol-7-amine (4E)

To the stirred solution of N-(1-(3,5-dichloropyridin-2-yl)ethyl)-6-fluoro-2-(methylthio)benzo[d]thiazol-7-amine (4C) (0.2 g, 0.51 mmol) in dichloromethane (5 mL) was added m-CPBA (0.18 g, 1.03 mmol) at 0° C. and the resulting reaction mixture was stirred at RT for 6 h. After the completion of reaction, the reaction mixture was quenched with saturated aq. NaHCO₃ solution and extracted with CH₂C₁₂(2 times). The organic layer was again washed with water, dried over anhydrous Na₂SO₄, filtered, and concentrated to get the crude compound (mixture of 4D and 4E) (0.24 g). LC-MS: 403.85 [M+H]⁺ and 420.1 [M+H]⁺.

Step-4: Synthesis of (1R,3r)-3-((3R)-3-(1-(7-((1-(3,5-dichloropyridin-2-yl)ethyl)amino)-6-fluorobenzo[d]thiazol-2-yl)azetidin-3-yl)piperidin-1-yl)-1-methylcyclobutane-1-carboxylic acid (Compound-35) A mixture of 4D (0.2 g, 0.49 mmol) and 4E (0.25 g, 0.99 mmol) in DMSO (3 mL) and was added CsF (0.24 g, 1.56 mmol) at 0° C. and the resulting reaction mixture was allowed to reach to RT and stirred at 100° C. for 16 h. After the completion of reaction, the reaction mixture was cooled to RT, quenched with saturated NH₄Cl solution, extracted with 10% MeOH in CH₂Cl₂. The organic layer was dried over anhydrous Na₂SO₄, filtered, and concentrated under reduced pressure to get crude compound which was purified by preparative TLC using 15% methanol in CH₂Cl₂ to afford the title compound 35 (0.1 g, 34%). LC-MS: 592.2 [M+H]⁺; ¹H-NMR (400 MHz, CD₃OD) δ 8.53 (d, 1H), 7.95-7.94 (dd, 1H), 7.07-7.02 (dd, 1H), 6.97-6.94 (dd, 1H), 5.39-5.37 (m, 1H), 4.33-4.30 (m, 2H), 4.08-4.05 (m, 2H), 3.85-3.75 (m, 1H), 3.60-3.55 (m, 1H), 3.45-3.34 (m, 1H), 2.92-2.82 (m, 2H), 2.80-2.72 (m, 2H), 2.54 (t, 1H), 2.27-2.21 (m, 2H), 2.15-1.98 (m, 2H), 1.83-1.70 (m, 1H), 1.48 (d, 3H), 1.46 (s, 3H), 1.30-1.15 (m, 1H). LC-MS: 592.2 [M+H]⁺

This racemic mixture was separated by using chiral HPLC and obtained both the isomers. The details of the method are as follows.
Column: CHIRALPAK IG (250 mm×4.6 mm, 5μ),
Mobile phase: A: n-Hexane B: 0.1% DEA in EtOH:05% TFA in water and B was acetonitrile
Flow rate: 15 mL/min.

(1R,3r)-3-((R)-3-(1-(7-(((R)-1-(3,5-dichloropyridin-2-yl)ethyl)amino)-6-fluorobenzo[d]thiazol-2-yl)azetidin-3-yl)piperidin-1-yl)-1-methylcyclobutane-1-carboxylic acid (Compound-36): LC-MS: 592.2 [M+H]⁺; ¹H-NMR (400 MHz, CD₃OD): δ 8.52 (d, 1H), 7.93 (d, 1H), 7.04-6.99 (dd, 1H), 6.98-6.93 (dd, 1H), 5.40-5.37 (m, 1H), 4.31-4,24 (m, 2H), 4.02-3.96 (m, 2H), 3.34-3.21 (m, 2H), 2.76-2.73 (m, 3H), 2.52-2.42 (m, 1H), 2.32-2.22 (m, 1H), 2.06-1.94 (m, 5H), 1.78-1.68 (m, 1H), 1.46 (d, 3H), 1.43 (s, 3H), 1.20-1.10 (m, 1H), 0.90 (m, 1H), HPLC:98.21% and chiral HPLC:93.35%.

(1R,3r)-3-((R)-3-(1-(7-(((S)-1-(3,5-dichloropyridin-2-yl)ethyl)amino)-6-fluorobenzo[d]thiazol-2-yl)azetidin-3-yl)piperidin-1-yl)-1-methylcyclobutane-1-carboxylic acid (Compound-37): LC-MS: 592.2 [M+H]⁺; ¹H-NMR (400 MHz, CD₃OD): δ 8.52 (d, 1H), 7.93 (d, 1H), 7.04-6.99 (dd, 1H), 6.98-6.93 (dd, 1H), 5.40-5.36 (m, 1H), 4.29-4,24 (m, 2H), 4.03-3.97 (m, 2H), 2.76-2.71 (m, 3H), 2.01-1.92 (m, 6H), 1.47 (d, 3H), 1.44-1.42 (m, 2H), 1.39 (s, 3H), 1.32-1.27 (m, 2H), 0.93-0.89 (m, 2H), HPLC: 97.16% and chiral HPLC 15:98.30%.

The below compounds were prepared using similar routes as Example-4 from the respective starting materials given as in Table-F with appropriate variations in coupling methods, reactants, quantities of reagents, and solvents, as required. The characterization data of the compounds are summarized herein the below table.

TABLE-F

| | | Route of | Starting | |
|---|---|---|---|---|
| Comp. No. | Structure | synthesis | material used | Spectral data |
| 38 | 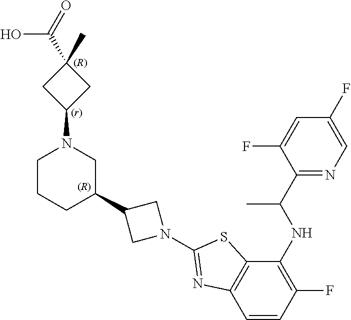 | Ex-4 | A-19 & A-25 | LC-MS: 560.2 [M + H]⁻; ¹H-NMR (400 MHz, CD₃OD) δ 8.36 (d, 1H), 7.57-7.52 (m, 1H), 7.21-7.15 (m, 1H), 6.94-6.91 (m, 1H) 5.26-5.20 (m, 1H), 4.52-4.47 (m, 2H), 4.36-4.28 (m, 2H), 3.82-3.74 (m, 1H), 3.56-3.50 (m, 1H), 3.48-3.42 (m, 1H), 2.87-2.72 (m, 4H), 2.59-2.50 (m, 1H), 2.44-2.29 (m, 3H), 2.12-1.97 (m, 2H), 1.94-1.82 (m, 1H), 1.58 (d, 3H), 1.48 (s, 3H), 1.30-1.18 (m, 1H). |
| 39 | 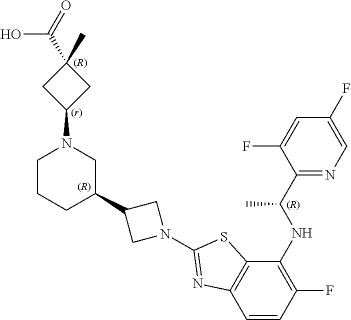 | Ex-4 | A-19 & A-25 | LC-MS: 560.1 [M + H]⁻; ¹H-NMR (400 MHz, CD₃OD) δ 8.38 (d, 1H), 7.54-7.49 (m, 1H), 7.03-6.93 (m, 2H), 5.28-5.22 (m, 1H), 4.31-4.25 (m, 2H), 4.03-3.98 (m, 2H), 3.24-3.15 (m, 1H), 2.78-2.69 (m, 3H), 2.45-2.36 (m, 1H), 2.20-2.10 (m, 2H), 2.07-1.87 (m, 6H), 1.76-1.64 (m, 1H), 1.52 (d, 3H), 1.39 (s, 3H), 1.21-1.09 (m, 1H). |
| | (Isomer-1 of compound 38) | | | |
| 40 | 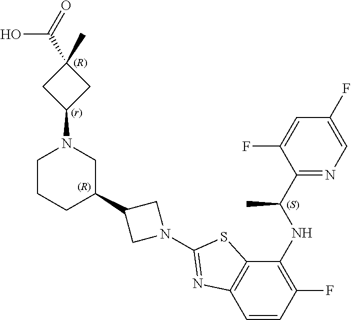 | Ex-4 | A-19 & A-25 | LC-MS: 560.1 [M + H]⁻; ¹H-NMR (400 MHz, CD₃OD) δ 8.38 (d, 1H), 7.54-7.49 (m, 1H), 7.04-6.94 (m, 2H), 5.29-5.18 (m, 1H), 4.31-4.26 (m, 2H), 4.04-3.99 (m, 2H), 3.52-3.50 (m, 1H), 3.42-3.24 (m, 1H), 2.80-2.75 (m, 3H), 2.64-2.53 (m, 1H), 2.38-2.29 (m, 1H), 2.09-1.95 (m, 6H), 1.82-1.73 (m, 1H), 1.52 (d, 3H), 1.41 (s, 3H), 1.22-1.15 (m, 1H). |
| | (Isomer-2 of compound 38) | | | |
| 41 | 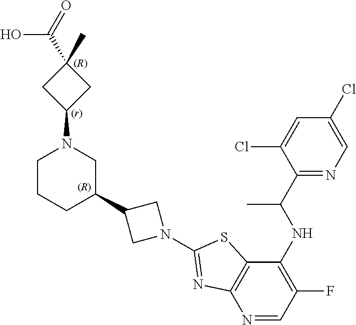 | Ex-4 | A-21 & I-11 | LC-MS: 593.2 [M + H]⁻; ¹H-NMR (400 MHz, CD₃OD) δ 8.51-8.49 (m, 1H), 8.04- 8.02 (m, 1H), 7.97 (d, 1H), 5.56-5.51 (m, 1H), 4.32-4.27 (m, 2H), 4.07-4.02 (m, 2H), 3.69-3.62 (m, 1H), 3.51-3.42 (m, 1H), 3.44-3.35 (m, 1H), 2.83-2.75 (m, 3H), 2.72-2.62 (m, 1H), 2.49-2.41 (m, 1H), 2.24-2.08 (m, 3H), 2.06-1.94 (m, 2H), 1.90-1.72 (m, 1H), 1.60 (d, 3H), 1.36 (s, 3H), 1.24-1.16 (m, 1H). |
| 42 | 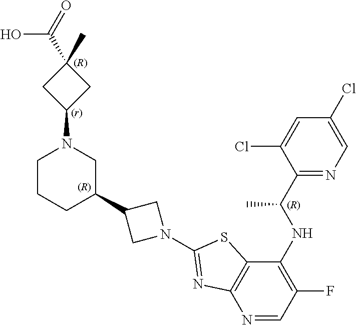 | Ex-4 | A-21 & I-11 | LC-MS: 593.1 [M + H]⁻; ¹H-NMR (400 MHz, CD₃OD) δ 8.50 (d, 1H), 8.02 (d, 1H), 7.93 (d, 1H), 5.56-5.51 (m, 1H), 4.28-4.24 (m, 2H), 4.01-3.99 (m, 2H), 3.18-3.02 (m, 3H), 2.71-2.66 (m, 3H), 2.26-2.14 (m, 1H), 1.95-1.88 (m, 6H), 1.76-1.66 (m, 1H), 1.60 (d, 3H), 1.38 (s, 3H), 1.12-1.02 (m, 1H). |
| | (Isomer-1 of compound 41) | | | |
| 43 | 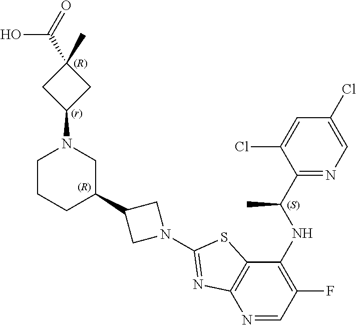 | Ex-4 | A-21 & I-11 | LC-MS: 593.1 [M + H]⁻; ¹H-NMR (400 MHz, CD₃OD) δ 8.50 (d, 1H), 8.02 (d, 1H), 7.95 (d, 1H), 5.55-5.51 (m, 1H), 4.31-4.25 (m, 2H), 4.06-3.98 (m, 2H), 3.49-3.38 (m, 1H), 3.28-3.10 (m, 1H), 2.78-2.71 (m, 3H), 2.50-2.39 (m, 1H), 2.26-2.16 (m, 1H), 2.08-1.92 (m, 6H), 1.78-1.69 (m, 1H), 1.60 (d, 3H), 1.38-1.34 (m, 3H), 1.18-1.09 (m, 1H). |
| | (Isomer-2 of compound 41) | | | |
| 44 | 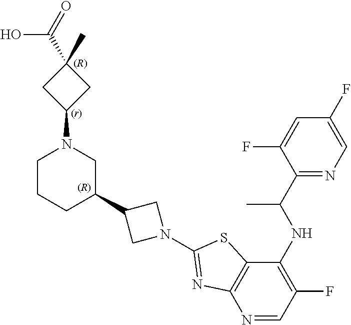 | Ex-4 | A-21 & A-25 | LC-MS: 561.2 [M + H]⁺; ¹H-NMR (400 MHz, CD₃OD) δ 8.36 (d, 1H), 7.95 (d, 1H), 7.63-7.58 (m, 1H), 5.47-5.42 (m, 1H), 4.31-4.28 (m, 2H), 4.06-4.00 (m, 2H), 3.38-3.33 (m, 2H), 3.32-3.23 (m, 1H), 2.76-2.71 (m, 3H), 2.45-2.42 (m, 1H), 2.26-2.15 (m, 1H), 2.08-1.92 (m, 5H), 1.70-1.68 (m, 1H), 1.61 (d, 3H), 1.34 (s, 3H), 1.20-1.08 (m, 1H). |
| 45 | 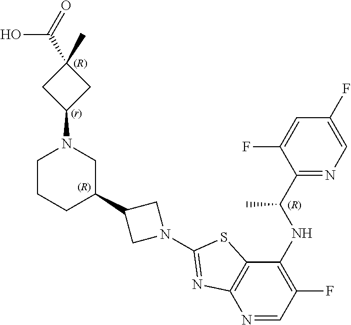 | Ex-4 | A-21 & A-25 | LC-MS: 561.1 [M + H]⁺; ¹H-NMR (400 MHz, CD₃OD) δ 8.36 (d, 1H), 7.94 (d, 1H), 7.61-7.60 (m, 1H), 5.46-5.44 (m, 1H), 4.30-4.26 (m, 2H), 4.04-4.00 (m, 2H), 3.34-3.32 (m, 3H), 2.74-2.70 (m, 3H), 2.40-2.32 (m, 1H), 2.00-1.90 (m, 6H), 1.78-1.65 (m, 1H), 1.61 (d, 3H), 1.42 (s, 3H), 1.35-1.30 (m, 1H). |
| | (Isomer-1 of compound 44) | | | |
| 46 | 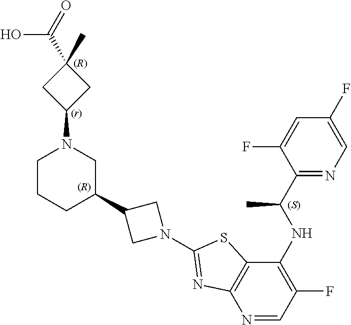 | Ex-4 | A-21 & A-25 | LC-MS: 561.2 [M + H]⁺; ¹H-NMR (400 MHz, CD₃OD) δ 8.36 (d, 1H), 7.94 (d, 1H), 7.63-7.58 (m, 1H), 5.46-5.44 (m, 1H), 4.29-4.27 (m, 2H), 4.03-4.01 (m, 2H), 3.34-3.32 (m, 3H), 2.74-2.69 (m, 3H), 2.40-2.32 (m, 1H), 2.25-1.85 (m, 6H), 1.79-1.65 (m, 1H), 1.61 (d, 3H), 1.42 (s, 3H), 1.35-1.30 (m, 1H). |
| | (Isomer-2 of compound 44) | | | |
| 47 | 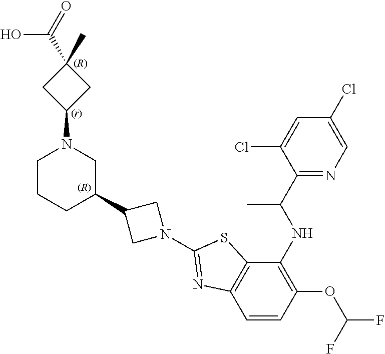 | Ex-4 | A-22 & I-11 | LC-MS: 640.2 [M + H]⁻; 1H-NMR (400 MHz, CD₃OD) δ 8.54 (d, 1H), 7.95 (d, 1H), 7.12 (d, 1H), 7.00-6.54 (m, 2H), 5.49-5.43 (m, 1H), 4.34-4.28 (m, 2H), 4.07-4.02 (m, 2H), 3.65-3.53 (m, 1H), 3.44-3.38 (m, 1H), 3.31-3.24 (m, 1H), 2.85-2.76 (m, 2H), 2.67-2.52 (m, 1H), 2.49-2.33 (m, 1H), 2.13-1.96 (m, 6H), 1.83-1.66 (m, 1H), 1.47 (d, 3H), 1.43 (s, 3H), 1.29-1.11 (m, 1H). |
| 48 | 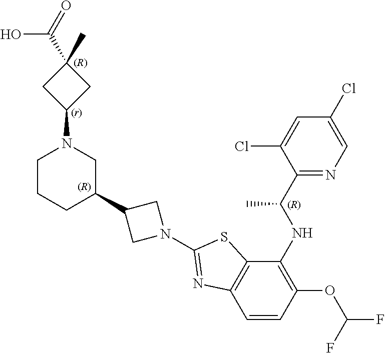 | Ex-4 | A-22 & I-11 | LC-MS: 640.15 [M + H]⁻; ¹H-NMR (400 MHz, CD₃OD) δ 8.54 (d, 1H), 7.95 (d, 1H), 7.12 (d, 1H), 7.00 (d, 1H), 6.91-6.54 (m, 1H), 5.49-5.41 (m, 1H), 4.30-4.27 (m, 2H), 4.04-4.00 (m, 2H), 3.53-3.42 (m, 1H), 3.29-3.22 (m, 2H), 2.79-2.72 (m, 2H), 2.60-2.48 (m, 1H), 2.35-2.24 (m, 1H), 2.09-1.92 (m, 6H), 1.82-1.68 (m, 1H), 1.47 (d, 3H), 1.41 (s, 3H), 1.23-1.11 (m, 1H). |
| 49 | 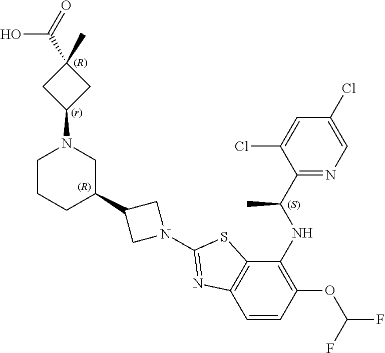 | Ex-4 | A-22 & I-11 | LC-MS: 640.15 [M + H]⁻; ¹H-NMR (400 MHz, CD₃OD) δ 8.54 (d, 1H), 7.95 (d, 1H), 7.12 (d, 1H), 7.00 (d, 1H), 6.92-6.52 (m, 1H), 5.48-5.42 (m, 1H), 4.32-4.26 (m, 2H), 4.16-4.00 (m, 2H), 3.59-3.53 (m, 1H), 3.46-3.38 (m, 3H), 2.81-2.72 (m, 3H), 2.64-2.54 (m, 1H), 2.45-2.32 (m, 1H), 2.17-2.01 (m, 3H), 2.00-1.94 (m, 1H), 1.84-1.74 (m, 1H), 1.47 (d, 3H), 1.42 (s, 3H), 1.21-1.15 (m, 1H). |
| | (Isomer-2 of compound 47) | | | |
| 50 | 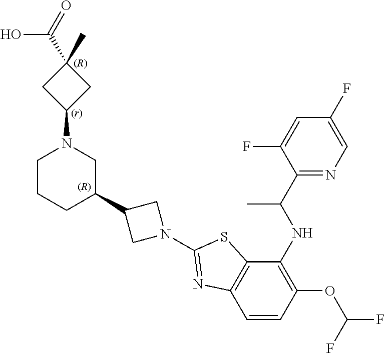 | Ex-4 | A-22 & A-25 | LC-MS: 608.2 |
| 51 | 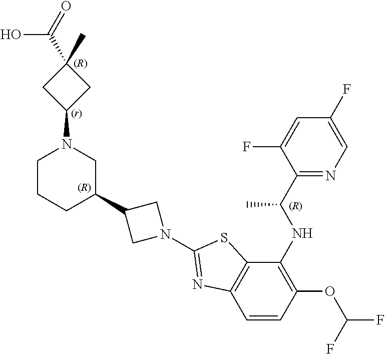 | Ex-4 | A-22 & A-25 | LC-MS: 608.2 [M + H]⁻; ¹H-NMR (400 MHz, CD₃OD) δ 8.38 (d, 1H), 7.56-7.51 (m, 1H), 7.11 (d, 1H), 7.00-6.50 (m, 2H), 5.34-5.26 (m, 1H), 4.36-4.25 (m, 2H), 4.09-4.01 (m, 2H), 3.51-3.42 (m, 2H), 3.28-3.17 (m, 1H), 2.79-2.70 (m, 3H), 2.55-2.38 (m, 1H), 2.28-2.19 (m, 1H), 2.07-1.92 (m, 5H), 1.79-1.68 (m, 1H), 1.49 (d, 3H), 1.40 (s, 3H), 1.25-1.12 (m, 1H). |
| | (Isomer-1 of compound 50) | | | |
| 52 | 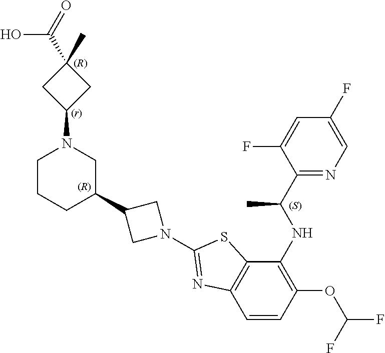 | Ex-4 | A-22 & A-25 | LC-MS: 608.2 [M + H]⁻; ¹H-NMR (400 MHz, CD₃OD) δ 8.38 (d, 1H), 7.55-7.51 (m, 1H), 7.11 (d, 1H), 7.01-6.57 (m, 1H), 6.52 (s, 1H), 5.32-5.25 (m, 1H), 4.36-4.27 (m, 2H), 4.09-4.00 (m, 2H), 3.45-3.37 (m, 1H), 3.32-3.28 (m, 1H), 2.82-2.75 (m, 3H), 2.70-2.59 (m, 1H), 2.46-2.36 (m, 1H), 2.18-2.04 (m, 4H), 2.02-1.95 (m, 2H), 1.85-1.74 (m, 1H), 1.50 (d, 3H), 1.43 (s, 3H), 1.27-1.12 (m, 1H). |
| | (Isomer-2 of compound 50) | | | |
| 53 | 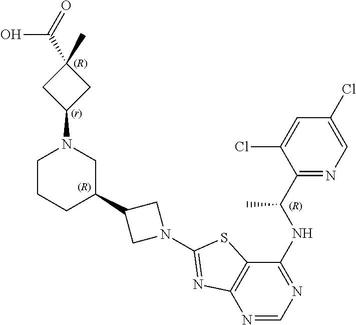 | Ex-4 | I-8 & I-11 | LC-MS: 576.1 [M + H]⁻; ¹H-NMR (400 MHz, CD₃OD) δ 8.46 (d, 1H), 8.18 (s, 1H), 7.97 (d, 1H), 5.87-5.81 (m, 1H), 4.31-4.27 (m, 2H), 4.04-4.00 (m, 2H), 2.92-2.82 (m, 2H), 2.73-2.65 (m, 2H), 2.63-2.59 (m, 2H), 1.91-1.85 (m, 2H), 1.76-1.71 (m, 4H), 1.69-1.62 (m, 1H), 1.60 (d, 3H), 1.52-1.38 (m, 1H), 1.35 (s, 3H), 1.32-1.28 (m, 1H). |

Although the present application as been illustrated by certain of the preceding examples, it is not to be construed as being limited thereby; but rather, the present application encompasses the generic area as hereinbefore disclosed. For example, the compounds below which can be prepared by following similar procedure as described in above Schemes/Examples with suitable modifications known to the one ordinary skilled in the art are also included in the scope of the present application:

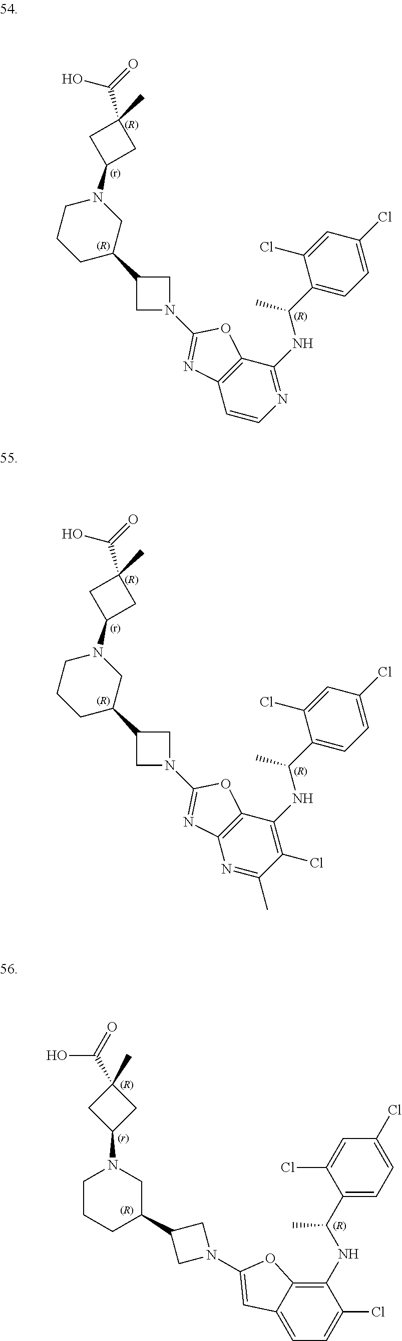

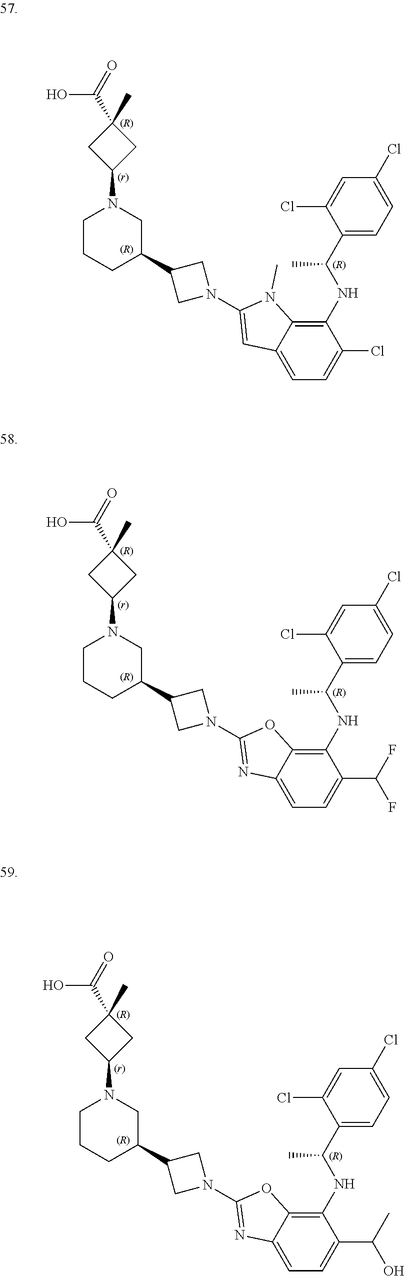

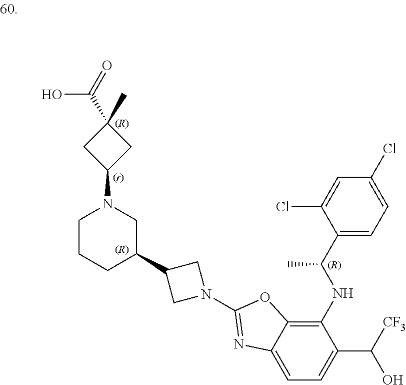

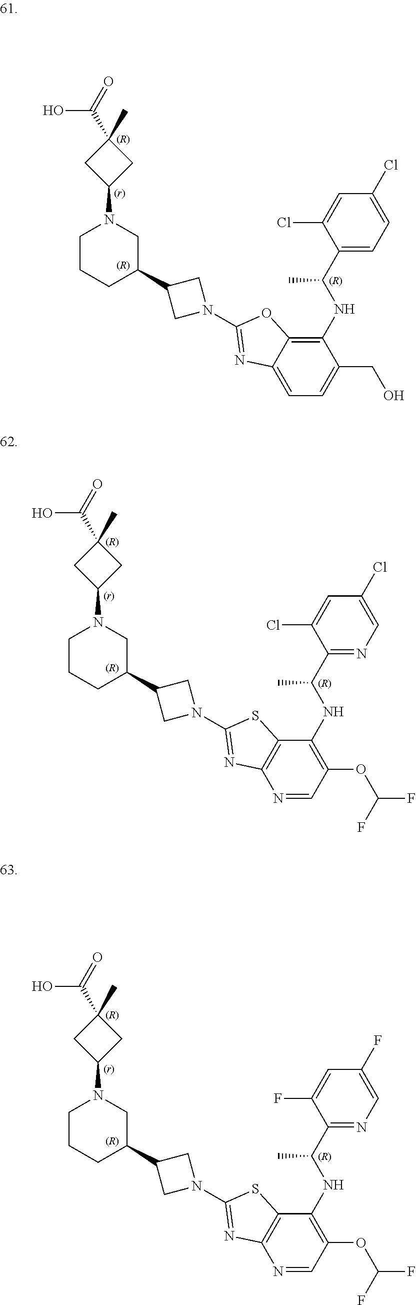

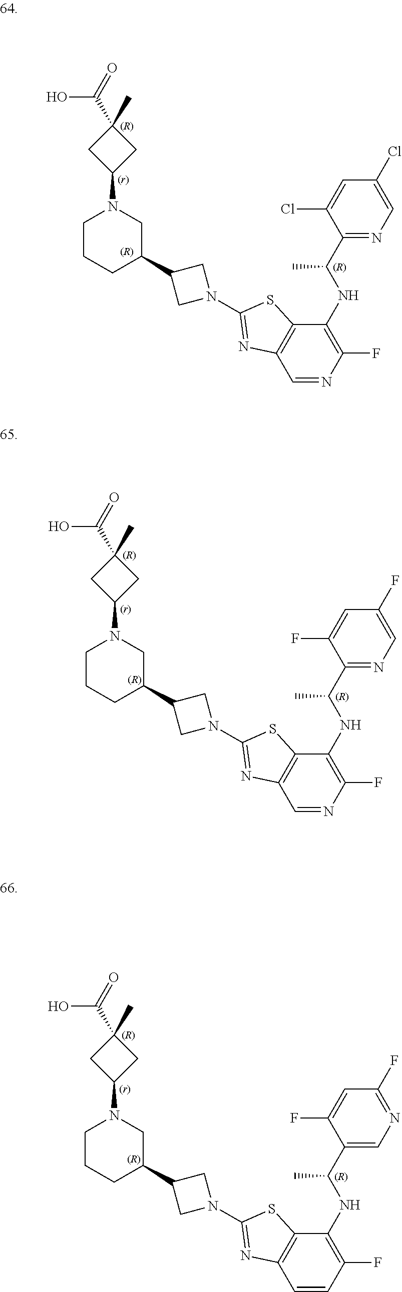

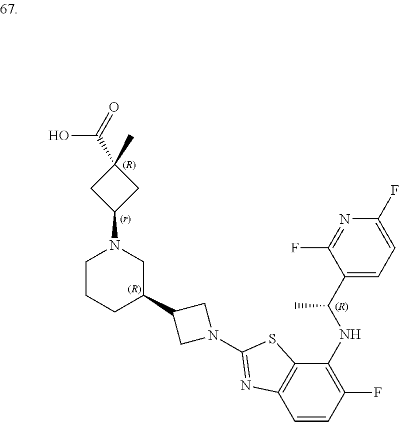

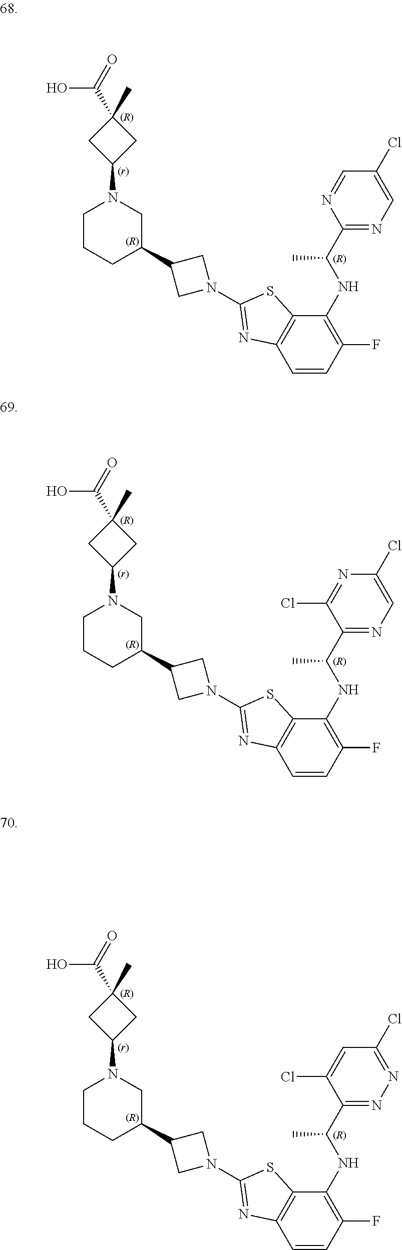

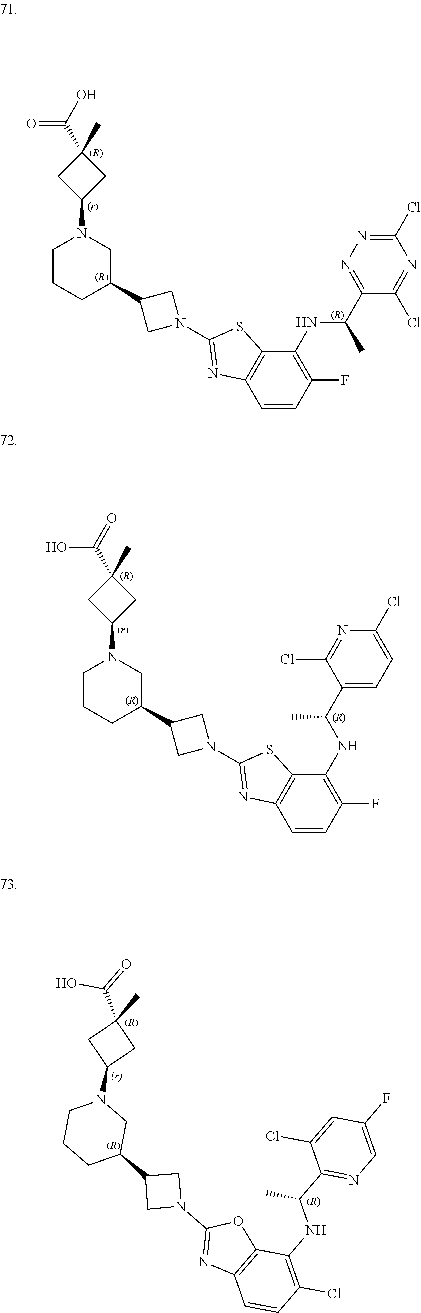

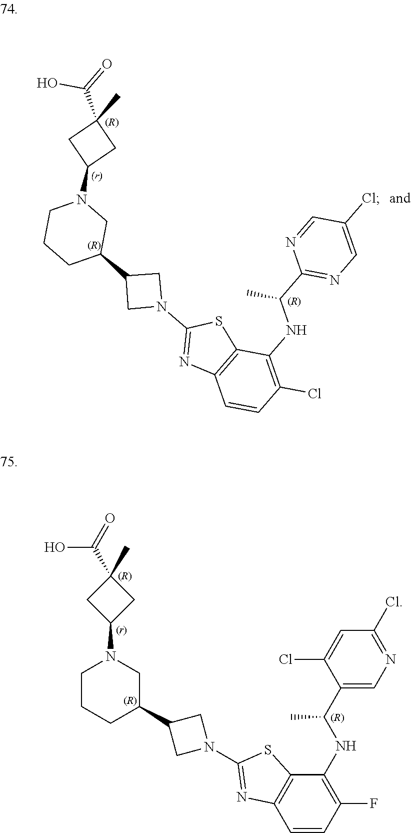

### Biology:

### β-Arrestin Recruitment Assay Protocol

CHO-K1 CCR4 β-Arrestin cells were seeded and incubated overnight at 37° C. in a CO₂ incubator for adherence. Next day, cells were pretreated with various concentrations of CCR4 modulators for 30 mins at 37° C. The cells were stimulated with recombinant human CCL22 (10 nM) and further incubated for 90 mins. Working detection solution was added and incubated at room temperature for 1 hour after which the chemiluminescent signal was read using a luminometer.

Exemplary compounds of the present application were screened by the above-mentioned assay and the results were tabulated.

The CCR4% inhibition values for certain exemplary compounds at 5 μM and at 30 μM are compiled in the table-G below.

TABLE G
% inhibition values of exemplary compounds

| Comp. | % inhibition at 5 μM/30 μM |
|---|---|
| No. | (% E-Max) |
| 1 | 103% at 30 μM |
| 2 | 99.5% at 30 μM |
| 4 | 71% at 30 μM |
| 5 | 102% at 30 μM |
| 6 | 104% at 30 μM |
| 7 | 101% at 30 μM |
| 8 | 102% at 5 μM |
| 9 | 103% at 5 μM |
| 10 | 93% at 5 μM |
| 11 | 101% at 5 μM |
| 12 | 99.3% at 5 μM |
| 13 | 101% at 5 μM |
| 14 | 96.4% at 5 μM |
| 15 | 101% at 5 μM |
| 16 | 103% at 5 μM |
| 17 | 102% at 5 μM |
| 18 | 103% at 5 μM |
| 19 | 93.8% at 5 μM |
| 20 | 41.5% at 5 μM |
| 21 | 103% at 5 μM |
| 22 | 29.5% at 30 μM |
| 23 | 104% at 5 μM |
| 24 | 61% at 5 μM |
| 25 | 101% at 5 μM |
| 26 | 103% at 5 μM |
| 27 | 99% at 5 μM |
| 28 | 72% at 5 μM |
| 29 | 103% at 5 μM |
| 30 | 103% at 5 μM |
| 31 | 19.3% at 5 μM |
| 32 | 104% at 5 μM |
| 33 | 103% at 5 μM |
| 34 | 102% at 5 μM |
| 35 | 102% at 5 μM |
| 36 | 103% at 5 μM |
| 37 | 78% at 5 μM |
| 38 | 81% at 5 μM |
| 39 | 39% at 5 μM |
| 40 | 84% at 5 μM |
| 41 | 101% at 5 μM |
| 42 | 103% at 5 μM |
| 43 | 95% at 5 μM |
| 44 | 99% at 5 μM |
| 45 | 89% at 5 μM |
| 46 | 99% at 5 μM |
| 47 | 93% at 5 μM |
| 48 | 52% at 5 μM |
| 49 | 99% at 5 μM |
| 51 | 77% at 30 μM |
| 52 | 99% at 30 μM |
| 53 | 94% at 5 μM |

The CCR4 antagonistic activity IC₅₀ values of some exemplary compounds are summarized below in table-H, wherein “A” refers to an IC₅₀ value less than 0.25 μM, “B” refers to an IC₅₀ value in range of 0.25 μM to 0.5 μM (both inclusive) and “C” refers to an IC₅₀ value greater than 0.5 μM.

TABLE H
IC₅₀ values of exemplary compounds

| IC₅₀ (μM) | |
|---|---|
| in range | Compounds |
| A | 1, 2, 6, 9, 11, 13, 16, 18, 21, 23, 25, 26, 29, 30, 32, 33, 35 and 36; |
| B | 5, 7, 8, 10, 12, 14, 15, 17, 19, 27, 34, 38, 40, 41, 42, 44, 46, 49 and 53; |
| C | 37, 43, 45, 47, 51 and 52. |

### INCORPORATION BY REFERENCE

All publications and patents mentioned herein are hereby incorporated by reference in their entirety as if each individual publication or patent were specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A compound of formula (I): 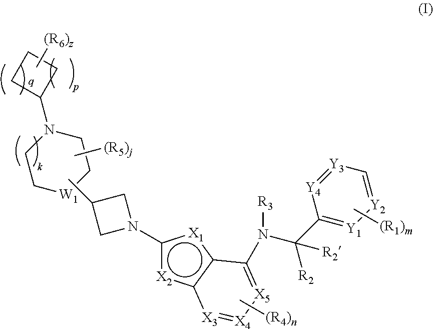
or a pharmaceutically acceptable salt thereof or a stereoisomer thereof;
wherein,
W₁ is C, N or O;
X₁ and X₂ are each independently CH, N, NRₓ, O, S or —S(O)—;
X₃, X₄ and X₅ are each independently C or N;
Rₓ is hydrogen or C₁-C₆ alkyl;
Y₁, Y₂, Y₃, and Y₄ are each independently C or N;
R₁ at each occurrence is independently C₁-C₆ alkyl, C₂-C₆ alkenyl, C₂-C₆ alkynyl, halo, cyano, amino, nitro, —OR₁ₐ, —C(O)R_{1b} or C₃-C₆ cycloalkyl; wherein each of C₁-C₆ alkyl, C₂-C₆ alkenyl, C₂-C₆ alkynyl, and C₃-C₆ cycloalkyl is optionally substituted with one or more substituents independently selected from halo, hydroxy and C₁-C₆ alkoxy;
R₂ and R₂′ are each independently hydrogen, C₁-C₆ alkyl or halo;
R₃ is hydrogen or C₁-C₆ alkyl;
R₄ at each occurrence is independently C₁-C₆ alkyl, C₂-C₆ alkenyl, C₂-C₆ alkynyl, halo, cyano, amino, nitro, —OR₄ₐ, —C(O)R_{4b}, or C₃-C₆ cycloalkyl; wherein each of C₁-C₆ alkyl, C₂-C₆ alkenyl, C₂-C₆ alkynyl, and C₃-C₆ cycloalkyl is optionally substituted with one or more substituents independently selected from one or more halo, hydroxy and C₁-C₆ alkoxy;
R₅ at each occurrence is independently halo, C₁-C₆ alkyl, C₁-C₆ haloalkyl, C₁-C₆ alkoxy or —C(O)R₅ₐ;
R₆ at each occurrence is independently C₁-C₆ alkyl, halo, C₁-C₆ haloalkyl, cyano, —C(O)R₆ₐ, —(C₁-C₃ alkyl)C(O)R₆ₐ or —OR_{6b};
R₁ₐ and R₄ₐ are each independently hydrogen, halo, C₁-C₆ alkyl, C₁-C₆ haloalkyl, C₃-C₆ cycloalkyl or C₃-C₆ heterocycloalkyl;
R_{1b}, R_{4b} and R₅ₐ are each independently hydrogen, C₁-C₆ alkyl, hydroxy, amino, C₁-C₆ alkylamino, C₁-C₆ haloalkyl, C₃-C₆ cycloalkyl or C₃-C₆ heterocycloalkyl;
R₆ₐ is hydrogen, hydroxy, C₁-C₆ alkyl, C₁-C₆ haloalkyl, C₁-C₆ alkoxy, C₁-C₆ haloalkoxy, amino, C₁-C₆ alkylamino or C₃-C₆ cycloalkyl;
R_{6b} is hydrogen, C₁-C₆ alkyl, C₁-C₆ haloalkyl or C₃-C₆ cycloalkyl;
‘m’ is selected from 0 to 5;
‘n’ is selected from 0 to 3;
‘j’ is selected from 0 to 4;
‘k’ is selected from 0 to 2;
‘p’ and ‘q’ are each independently selected from 1 and 2; and
‘z’ is selected from 0 to 4.

2. The compound of claim 1, having formula (IA): 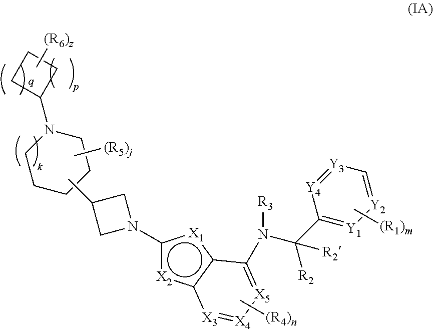
or a pharmaceutically acceptable salt thereof or a stereoisomer thereof.

3. The compound of claim 1, having formula (IB): 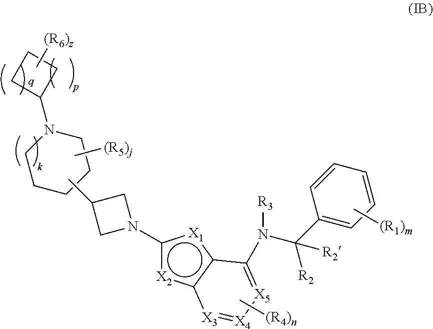
or a pharmaceutically acceptable salt thereof or a stereoisomer thereof.

4. The compound of claim 1, having formula IC to (IL): 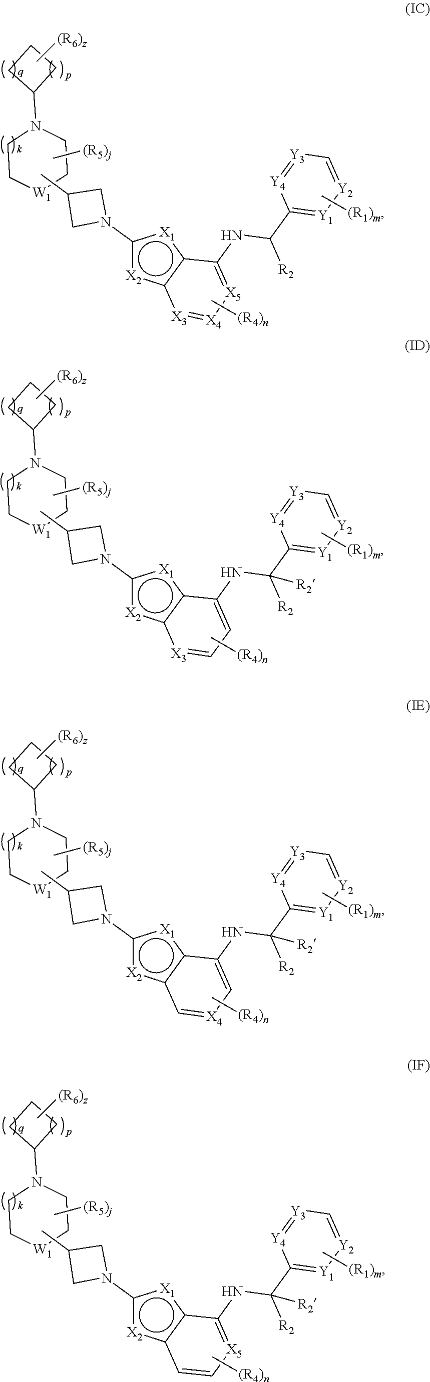 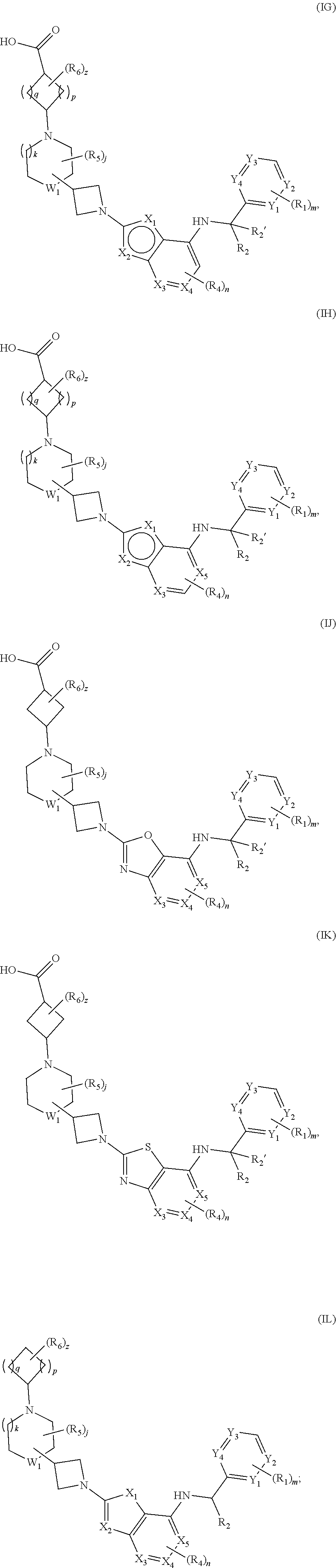
or a pharmaceutically acceptable salt thereof or a stereoisomer thereof.

5-6. (canceled)

7. The compound of claim 1, wherein the ring represented by 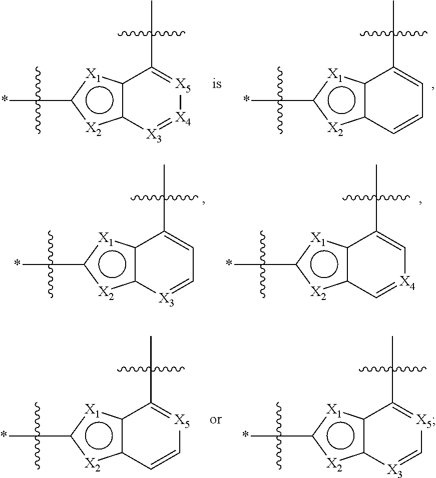
wherein the asterisk mark represents the point of attachment with azetidine ring.

8. The compound of claim 1, wherein the ring represented by, 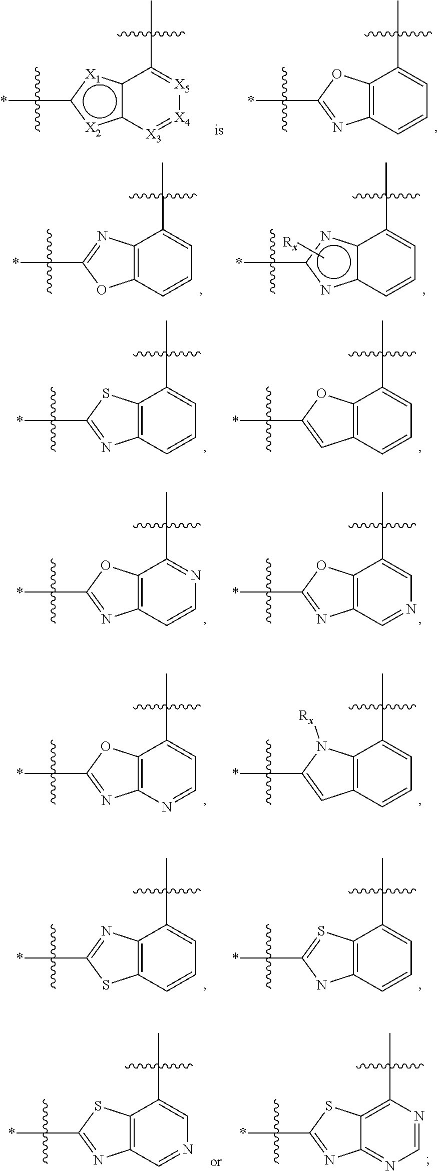
wherein the asterisk mark represents the point of attachment with azetidine ring.

9. The compound of claim 1, wherein the ring represented by, 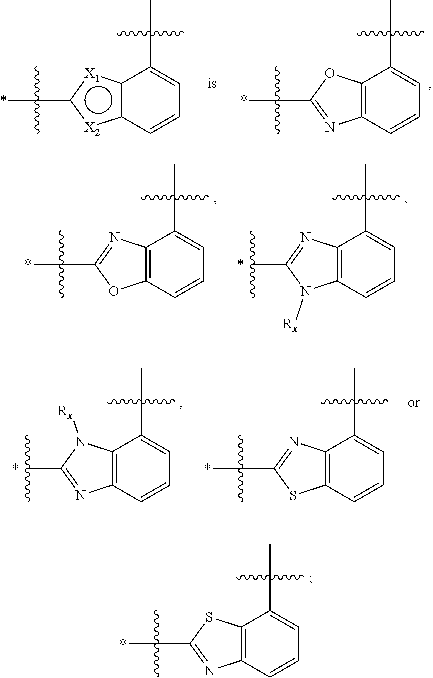
wherein the asterisk mark represents the point of attachment with azetidine ring.

10. The compound of claim 1, wherein the ring 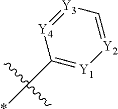
is independently 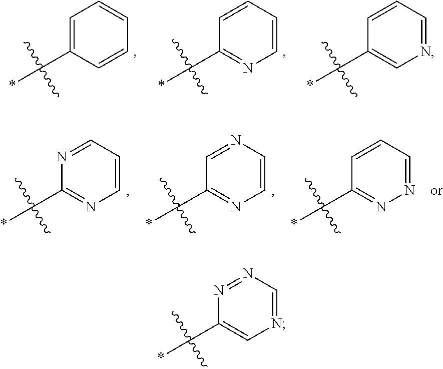
wherein asterisk mark represents point of attachment with carbon atom having R₂ and R₂′ groups.

11-12. (canceled)

13. The compound of claim 1, wherein X₁ is N, O or S, and X₂ is N, and R₁ at each occurrence is halo.

14-22. (canceled)

23. The compound of claim 1, wherein R₂ is C₁-C₆ alkyl, R₂′ is hydrogen, R₃ is hydrogen, R₄ is halo, and R₆ at each occurrence is independently selected from —CH₃ and —C(O)OH.

24-26. (canceled)

27. A compound selected from:

| Comp. | |
|---|---|
| No. | Structure |
| 1 | 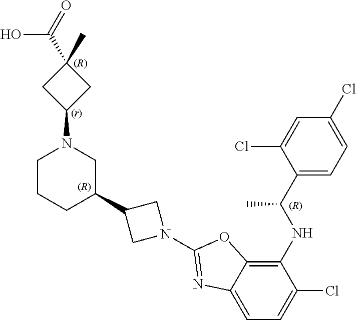 |
| 2 | 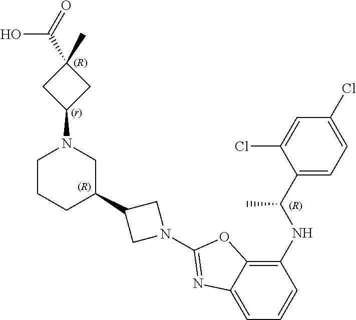 |
| 3 | 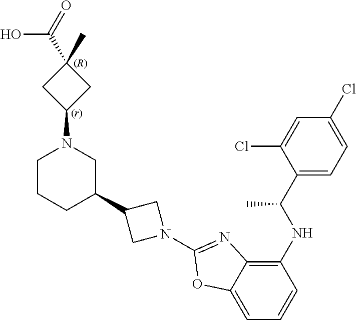 |
| 4 | 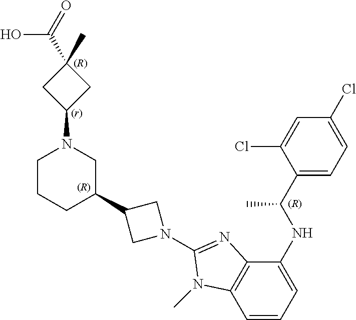 |
| 5 | 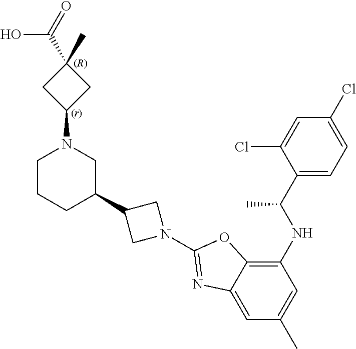 |
| 6 | 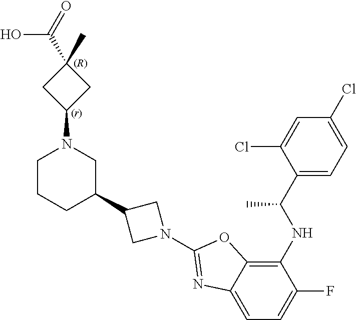 |
| 7 | 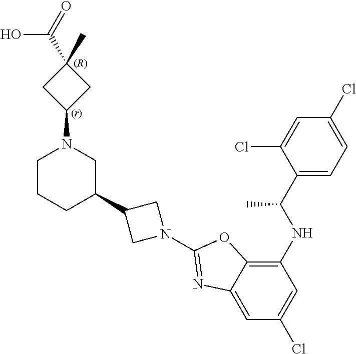 |
| 8 | 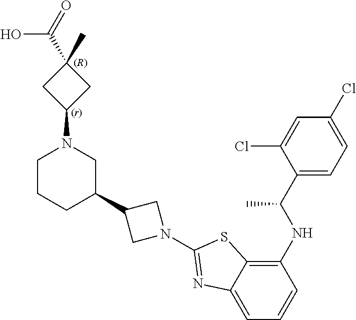 |
| 9 | 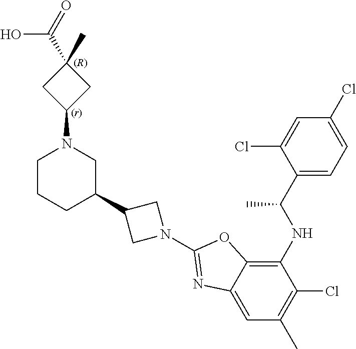 |
| 10 | 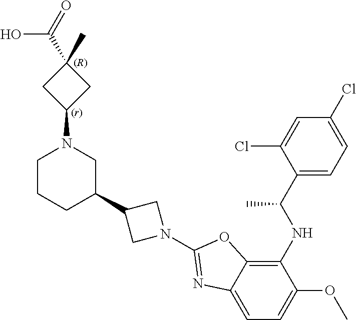 |
| 11 | 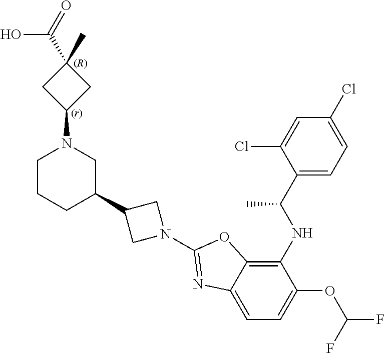 |
| 12 | 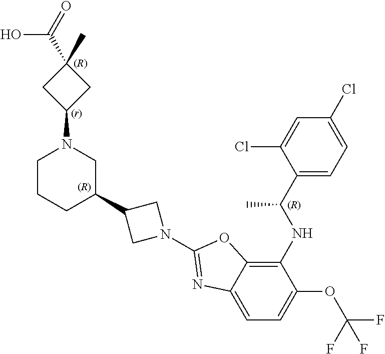 |
| 13 | 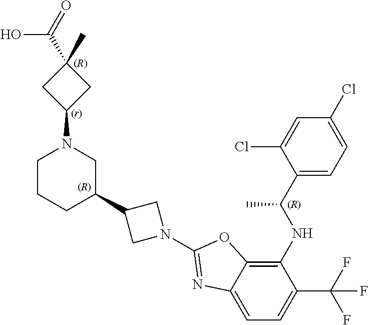 |
| 14 | 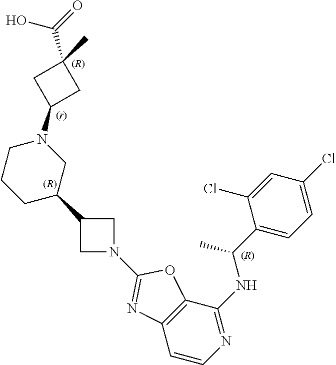 |
| 15 | 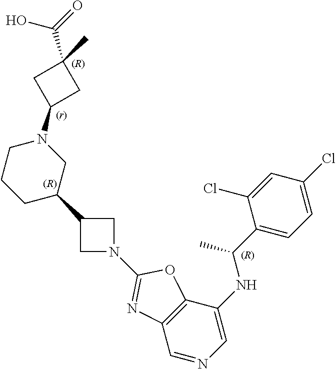 |
| 16 | 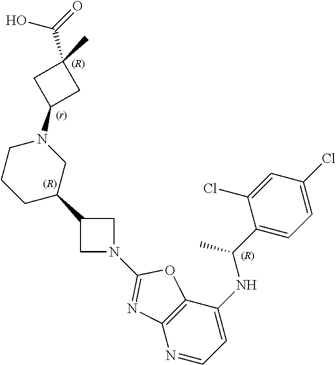 |
| 17 | 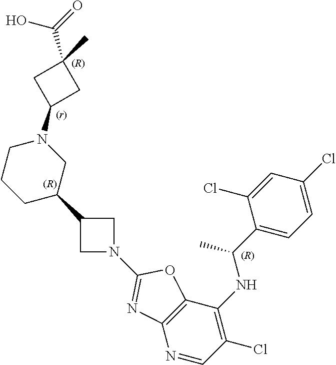 |
| 18 | 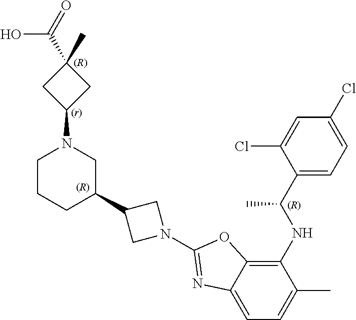 |
| 19 | 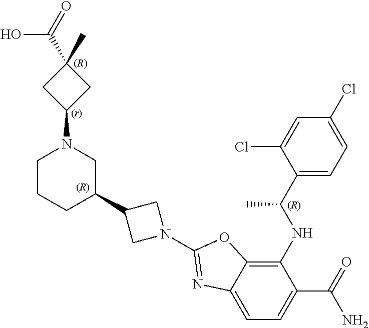 |
| 20 | 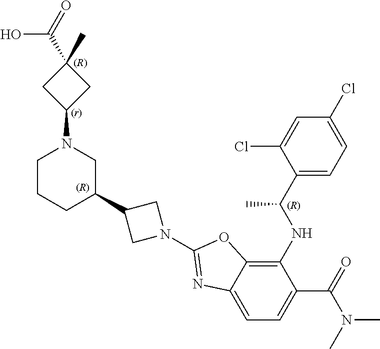 |
| 21 | 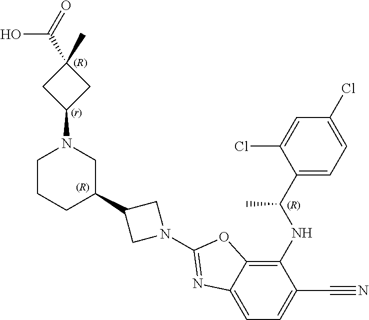 |
| 22 | 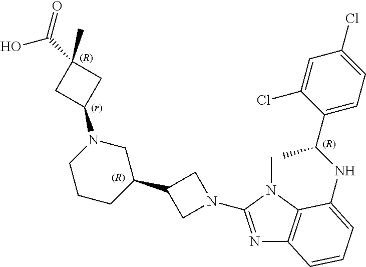 |
| 23 | 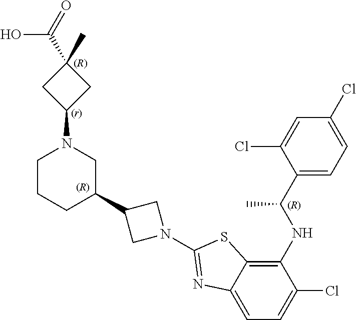 |
| 24 | 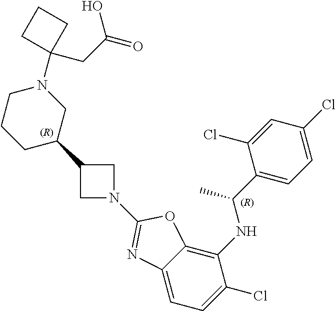 |
| 25 | 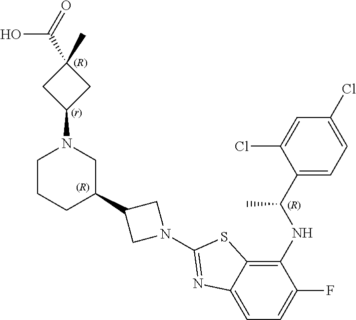 |
| 26 | 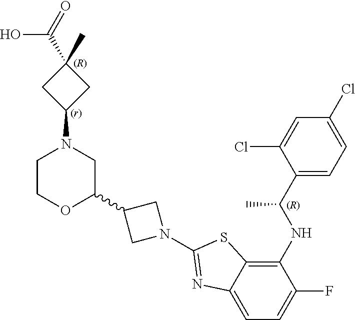 |
| 27 | 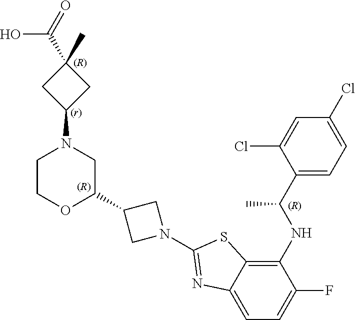 |
| 28 | 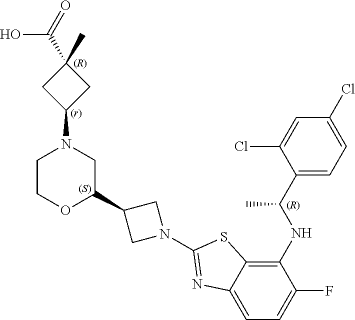 |
| 29 | 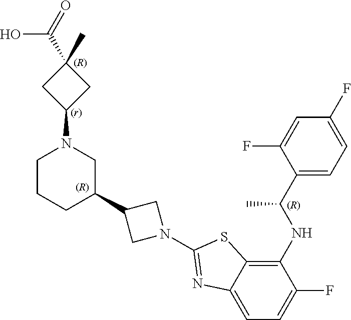 |
| 30 | 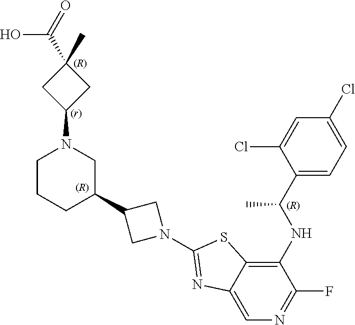 |
| 31 | 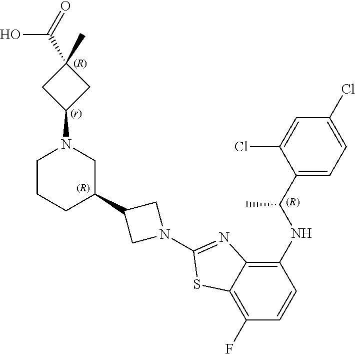 |
| 32 | 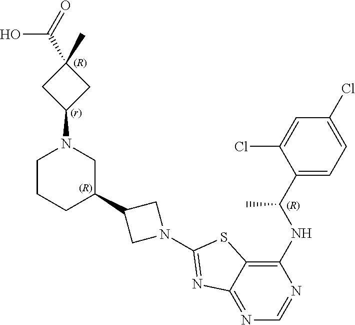 |
| 33 | 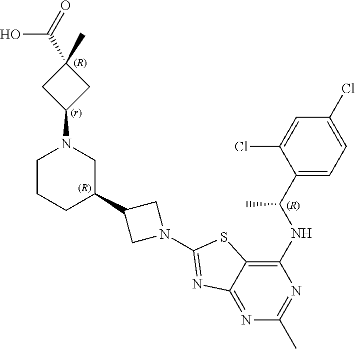 |
| 34 | 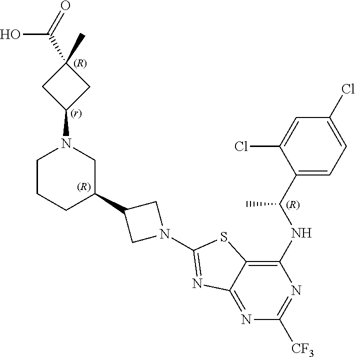 |
| 35 | 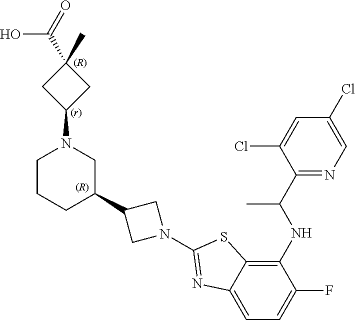 |
| 36 | 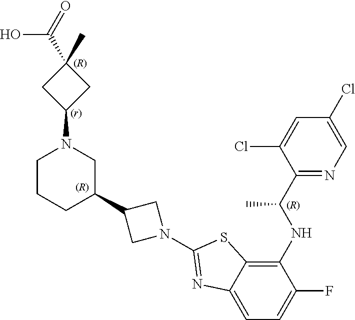 |
| | Isomer-1 of compound 35 |
| 37 | 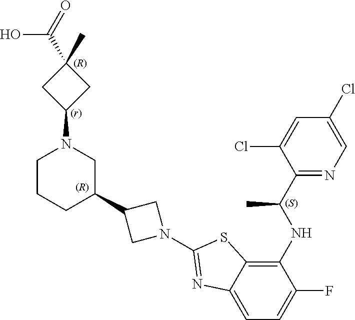 |
| | Isomer-2 of compound 35 |
| 38 | 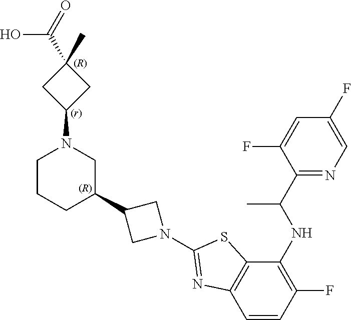 |
| 39 | 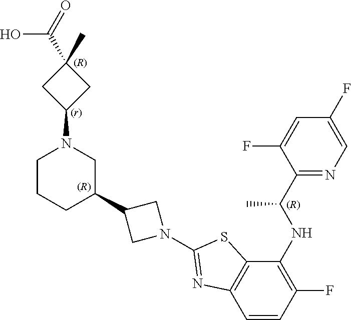 |
| | Isomer-1 of compound 38 |
| 40 | 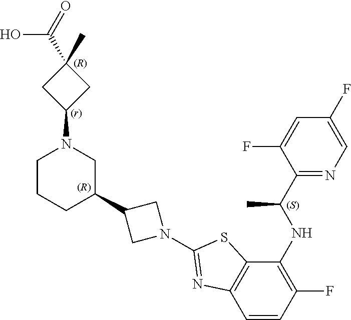 |
| | Isomer-2 of compound 38 |
| 41 | 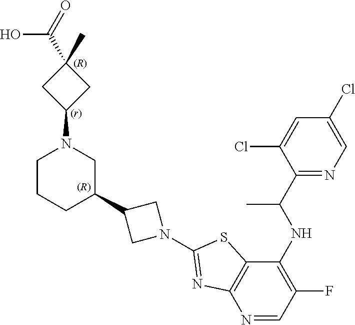 |
| 42 | 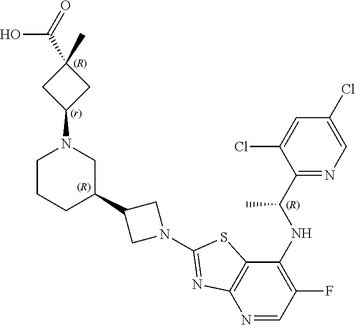 |
| | Isomer-1 of compound 41 |
| 43 | 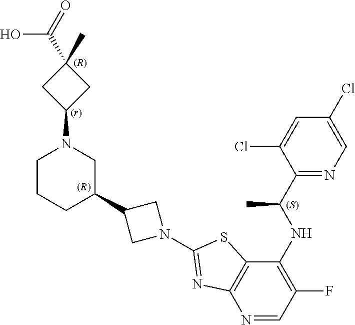 |
| | Isomer-2 of compound 41 |
| 44 | 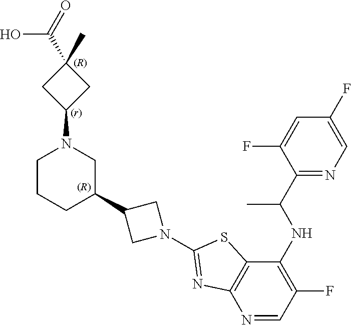 |
| 45 | 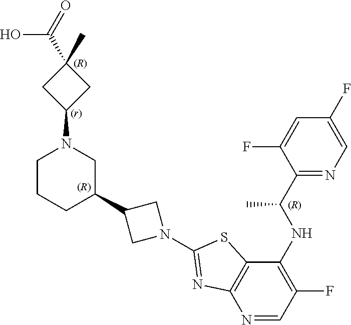 |
| | Isomer-1 of compound 44 |
| 46 | 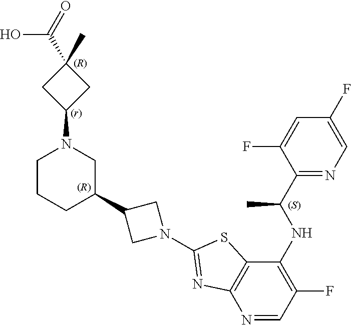 |
| | Isomer-2 of compound 44 |
| 47 | 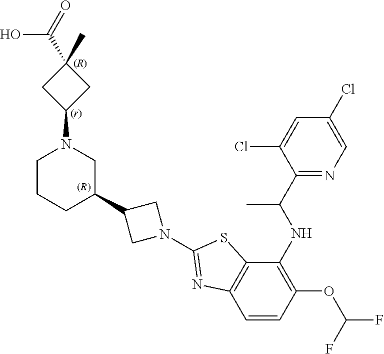 |
| 48 | 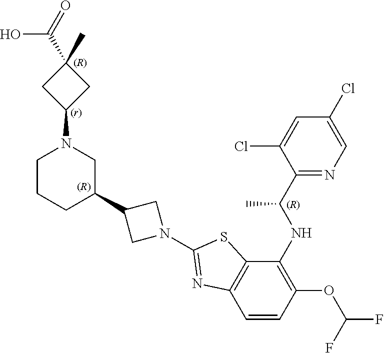 |
| | Isomer-1 of compound 47 |
| 49 | 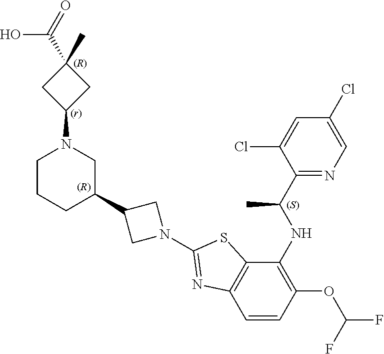 |
| | Isomer-2 of compound 47 |
| 50 | 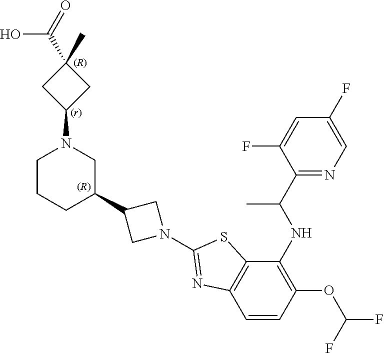 |
| 51 | 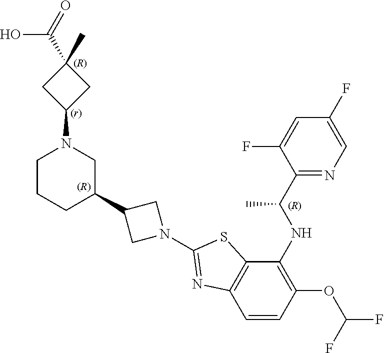 |
| | Isomer-1 of compound 50 |
| 52 | 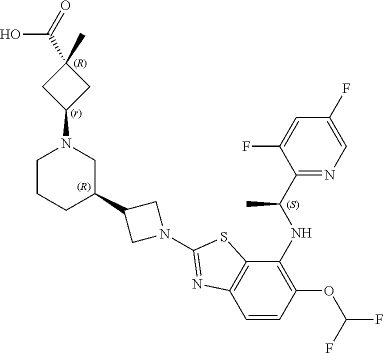 |
| | Isomer-2 of compound 50 |
| 53 | 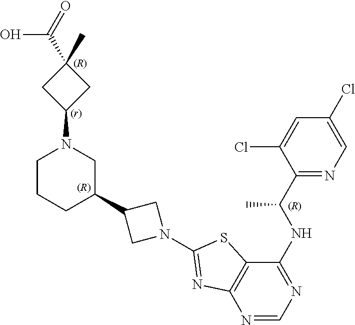 |
| 54 | 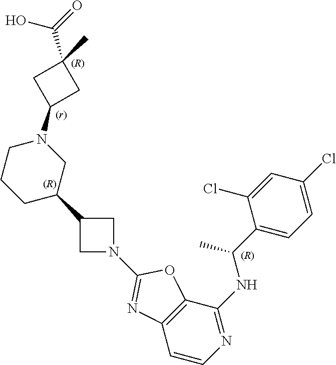 |
| 55 | 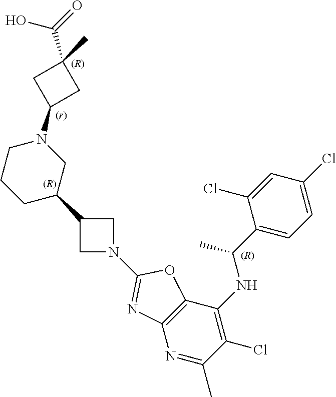 |
| 56 | 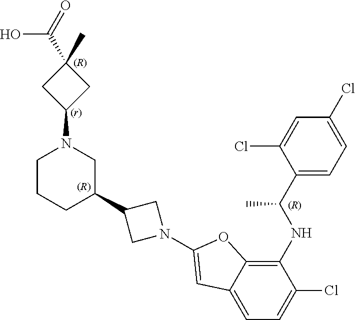 |
| 57 | 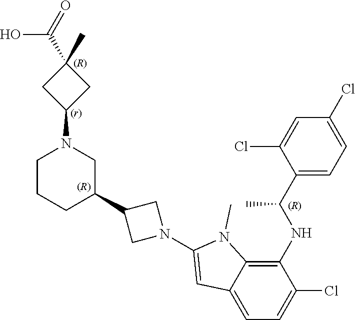 |
| 58 | 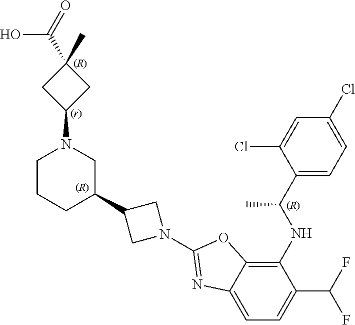 |
| 59 | 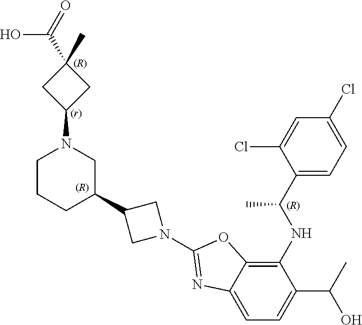 |
| 60 | 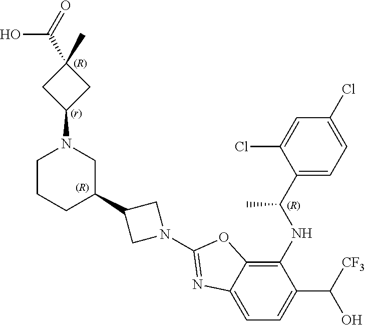 |
| 61 | 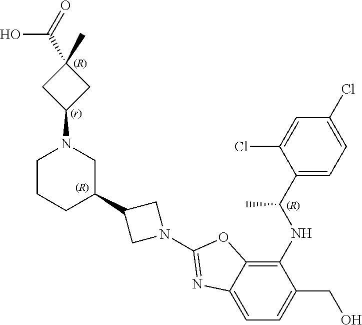 |
| 62 | 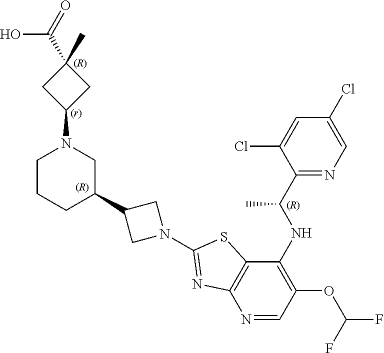 |
| 63 | 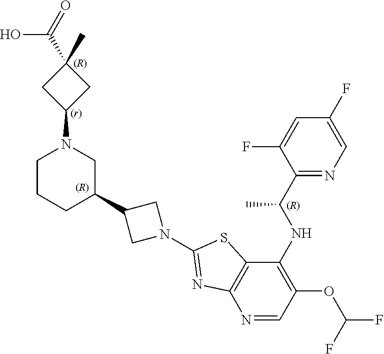 |
| 64 | 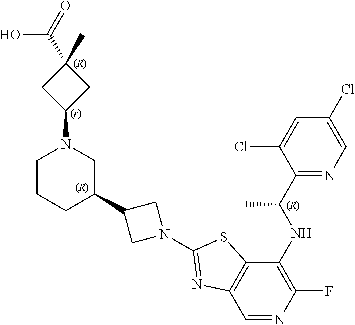 |
| 65 | 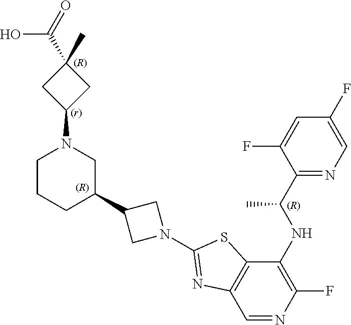 |
| 66 | 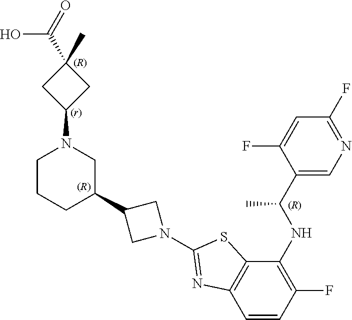 |
| 67 | 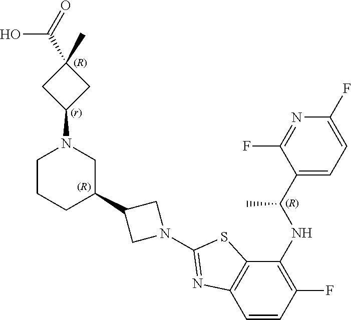 |
| 68 | 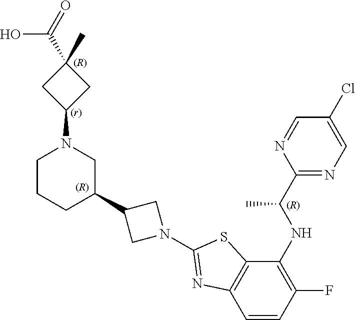 |
| 69 | 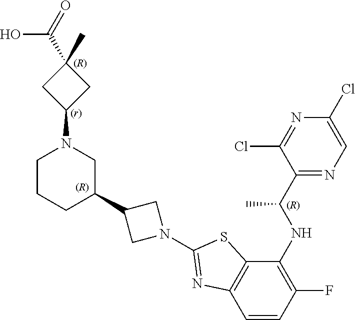 |
| 70 | 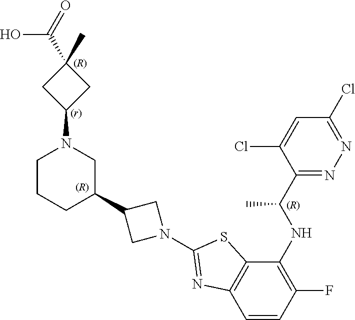 |
| 71 | 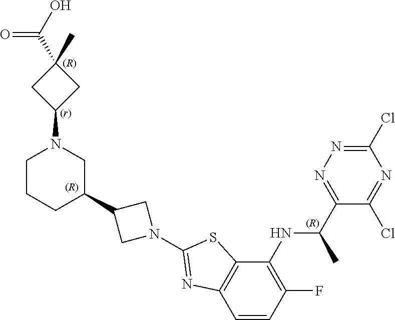 |
| 72 | 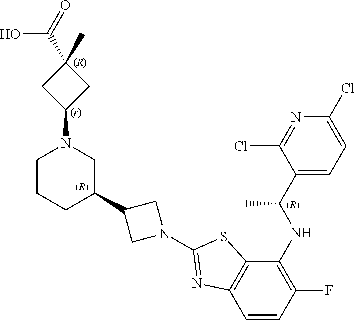 |
| 73 | 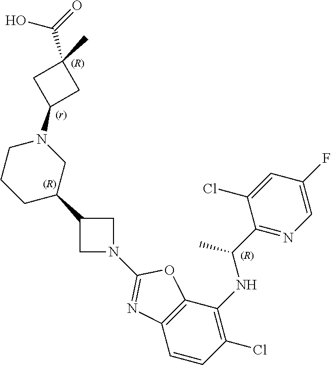 |
| 74 | 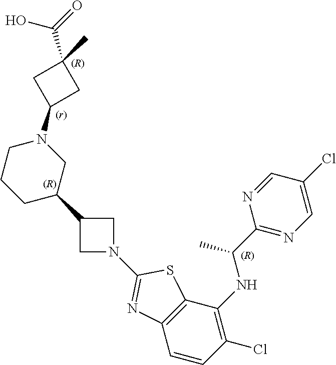 |
| 75 | 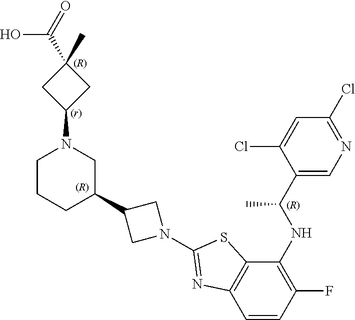 |
| — | — |

or a pharmaceutically acceptable salt or a stereoisomer thereof.

28. A pharmaceutical composition comprising a compound of claim 1, or a pharmaceutically acceptable salt or a stereoisomer thereof and at least one pharmaceutically acceptable carrier or excipient.

29. (canceled)

30. The compound of claim 1, or a pharmaceutical acceptable salt or a stereoisomer thereof, for use in the treatment of CCR4 mediated disease or disorder.

31. A method of modulating CCR4 in a subject, comprising administering to the subject with the compound of claim 1, or a pharmaceutically acceptable salt thereof.

32. A method of treating or preventing a disease or disorder mediated by CCR4 in a subject in need thereof, comprising administering to the subject a therapeutically effective amount of the compound of claim 1, or a pharmaceutically acceptable salt thereof, wherein the disease or disorder is cancer, an inflammation, dermatitis, atopic dermatitis or contact dermatitis.

33-37. (canceled)

38. The method of claim 32, wherein the cancer is colon cancer, pancreatic cancer, intestinal cancer, breast cancer, lung cancer, gastric cancer, liver cancer or colorectal cancer.

39. The method of claim 31, further comprising administering to the subject a therapeutically effective amount of a compound of claim 1 along with one or more chemotherapeutic agents or anti-inflammatory agents.

40-44. (canceled)

45. A method of treating or preventing an inflammatory disease or disorder in a subject in need thereof, comprising administering to the subject a therapeutically effective amount of the compound of claim 1, or a pharmaceutically acceptable salt thereof, wherein the inflammatory disease or disorder is inflammation, dermatitis, atopic dermatitis or contact dermatitis.

46-48. (canceled)

49. A method of treating or preventing cancer in a subject in need thereof, comprising administering to the subject a therapeutically effective amount of the compound of claim 1, or a pharmaceutically acceptable salt thereof, wherein the cancer is colon cancer, pancreatic cancer, intestinal cancer, breast cancer, lung cancer, gastric cancer, liver cancer or colorectal cancer.

50. (canceled)

51. The method of claim 32, further comprising administering to the subject a therapeutically effective amount of a compound of claim 1 along with one or more chemotherapeutic agents or anti-inflammatory agents.
